(19) United States
(12) Patent Application Publication
(10) Pub. No.: US 2003/0036683 A1
(43) Pub. Date: Feb. 20, 2003

(54) Method, system and computer program product for internet-enabled, patient monitoring system

(76) Inventors: Bruce A. Kehr, Potomac, MD (US); Robert H. Benson, Bethesda, MD (US)

Correspondence Address: Robert H. BENSON, INFORMEDIX, INC., 5920 Hubband Drive, Rockville, MD, 20852, US

(21) Appl. No.: 09/845,066

(22) Filed: May 7, 2001

Related U.S. Application Data
(60) Provisional application No. 60/200,853, filed on May 1, 2000.
(60) Provisional application No. 60/214,688, filed on Jun. 27, 2000.
(60) Provisional application No. 60/226,208, filed on Aug. 18, 2000.
(60) Provisional application No. 60/226,515, filed on Aug. 21, 2000.
(60) Provisional application No. 60/227,785, filed on Aug. 25, 2000.
(60) Provisional application No. 60/228,360, filed on Aug. 28, 2000.
(60) Provisional application No. 60/230,367, filed on Sep. 6, 2000.
(60) Provisional application No. 60/231,828, filed on Sep. 12, 2000.
(60) Provisional application No. 60/241,672, filed on Oct. 19, 2000.
(60) Provisional application No. 60/248,390, filed on Nov. 14, 2000.
(60) Provisional application No. 60/260,231, filed on Jan. 8, 2001.
(60) Provisional application No. 60/266,430, filed on Feb. 5, 2001.

(51) Int. Cl. A61B 5/00

(52) U.S. Cl. 600/300

(57) ABSTRACT

The present invention describes methods and apparatus useful in remotely modifying medical protocols by subgroups that are defined by specific database characteristics. In addition individual patient protocols may also be remotely modified. The method has application in medical databases containing the data collected by remote device monitoring of outpatients. These outpatients may be managed by one or more medical treatment or clinical research protocols or regimens involving pharmaceutical drugs, physiological data, educational content, and health status assessment or quality of life questionnaires. This method of mass customization of medical protocols, present in a medical database, is useful in efficiently monitoring and managing medical outpatients, through simultaneously instructing selected patient subgroups present in the database to make a change in their respective medical protocols. These changes are to be applied to all members of the selected medical subgroup; and in addition the method provides for specific patient instructions for the individualized components of the medical protocols. By using the mass customization method whereby subgroup selected changes are combined with individualized changes, both population management and individual patient management goals may be realized

The present invention further describes methods and apparatus useful in translating a complex medical treatment plan of a medical outpatient into a sequential series of automated, prompt and record events presented over time. In addition, individual patient medical treatment plans may be remotely created, modified, or viewed depending upon role-based assignments that permit different levels of access to and modification of the patient's time-and-event-driven medical treatment plan, depending upon the assigned role of the caregiver in the patient's treatment. The method has application in creating and linking medical databases containing data points that define the outpatient's medical treatment plan or protocol, with devices that can prompt the outpatient to carry out the sequential steps of a medical treatment plan, in proximity to the database, or while mobile at remote locations from the database.

This method facilitates converting a complex medical treatment plan into a series of simple steps presented by the remote prompting and monitoring device, to assist patients and their caregivers in proper health management. By using the real-time, time-and-event-driven method, individual patient management goals and improved patient treatment outcomes may be realized by outpatients with chronic and complex conditions.

### BACKGROUND OF THE INVENTION

1. Field of the Invention

The present invention relates generally to an improved health status and pharmaceutical compliance monitoring system, software, database and device, and more particularly to the Med-eMonitor system described.

2. Background Art

The prior art discloses a number of electronic devices that assist with the administration of prescribed medication and monitor the medical treatment progress. Medication and medical monitoring devices such as those disclosed in U.S. Pat. Nos. 5,200,891 and 5,642,731 provide a number of functions for facilitating patient adherence to prescribed therapies, and for facilitating cross-correlation of compliance data and clinical information about the patient. Some of such devices; have a plurality of medication compartments, a microprocessor with associated circuitry for providing timing signals, signals and display messages and for reading inputs from buttons that convey programming and operating information. Other devices lack medication compartments. They rely on programmed schedules for providing audible and/or visual alert signals at the scheduled times for taking certain medications and indicate the specific compartment from which the particular medication is to be taken, and quantity to be taken. The device of U.S. Pat. No. 5,642,731 is also capable of collecting contemporaneous data concerning the patient's adherence to the medication schedule, the progression of the medical condition(s) being treated, symptoms and side effects the patient is experiencing and other information pertinent to monitoring and treating the patient's medical condition(s).

However, the prior art suffers from a number of inherent disadvantages that have been solved by the present invention. While the prior art provides a medical monitoring device, operating system and communication systems generally, it does not have a system for the mass customizing of patient protocols and regimens that is simple to use. The prior art disclosed a device containing a dedicated key, button, or touch point on a screen associated with each medication being administered or disease being treated. Each of the dedicated keys facilitates accessing information related to the associated medication or disease (i.e. electronic prescription labeling, pictures and color of the medication, description of the medication's function, conditions or diseases that the medication treats, side effects associated with the medication, drugs with which the medication interacts, etc.). An additional set of soft keys, buttons or touch points facilitates additional functionality (i.e. flexible applications, programming, switching between modes of operation, response to patient queries, patient entry of physiologic values, accessing additional information regarding unscheduled and missed doses, communicating with remote devices, scrolling through additional messages, etc.).

### BRIEF SUMMARY OF THE INVENTION

The present invention provides a medical information management system and database. It provides for a number of enhanced features including: mass customization of medical protocols; time-and-event driven medical treatment plan; risk-stratified triage and medical intervention system; self-selected synchronized database-linked medical monitoring system; pharmacoeconomic analysis system; creation and self-selection of musical alarms for monitoring; streaming video and pictorial representation on monitoring device; a system for promoting enhanced validity in pharmaceuticals and drug package inserts; a system for mass customizing information device functions and features; and a dynamic, mass customizable, interactive screen and voice system for monitoring.

### BRIEF DESCRIPTION OF THE DRAWINGS/FIGURES

The accompanying drawings, which are incorporated herein and form part of the specification, illustrate the present invention and, together with the description, further serve to explain the principles of the invention and to enable a person skilled in the pertinent art to make and use the invention. In the drawings, like reference numbers indicate identical or functionally similar elements. Additionally, the left-most digit(s) of a reference number identifies the drawing in which the reference number first appears.

FIG. 1 illustrates a block diagram of the configuration of a healthcare system according to an embodiment of the present invention.

FIG. 2 illustrates a medical monitoring device according to an embodiment of the present invention.

FIG. 3 illustrates a block diagram of the configuration of an information management system and patient database according to an embodiment of the present invention.

FIG. 4 illustrates a graphical user interface (GUI) according to an embodiment of the present invention.

FIG. 5 illustrates a GUI according to an embodiment of the present invention.

FIG. 6 illustrates a GUI according to an embodiment of the present invention.

FIG. 7 illustrates a GUI according to an embodiment of the present invention.

FIG. 8 illustrates a GUI according to an embodiment of the present invention.

FIG. 9 illustrates a GUI according to an embodiment of the present invention.

FIG. 10 illustrates a GUI according to an embodiment of the present invention.

FIG. 11 illustrates a GUI according to an embodiment of the present invention.

FIG. 12 illustrates a GUI according to an embodiment of the present invention.

FIG. 13 illustrates a GUI according to an embodiment of the present invention.

FIG. 14 illustrates a GUI according to an embodiment of the present invention.

FIG. 15 illustrates a GUI according to an embodiment of the present invention.

FIG. 16 illustrates a GUI according to an embodiment of the present invention,

FIG. 17 illustrates a GUI according to an embodiment of the present invention.

FIG. 18 illustrates a GUI according to an embodiment of the present invention.

FIG. 19 illustrates a GUI according to an embodiment of the present invention.

FIG. 20 illustrates a GUI according to an embodiment of the present invention.

FIG. 21 illustrates a GUI according to an embodiment of the present invention.

FIG. 22 illustrates a GUI according to an embodiment of the present invention.

FIG. 23 illustrates a GUI according to an embodiment of the present invention.

FIG. 24 illustrates a GUI according to an embodiment of the present invention.

FIG. 25 illustrates a GUI according to an embodiment of the present invention.

FIG. 26 illustrates an operational flow diagram for the steps involved in customizing medical protocols.

FIG. 27 is a block diagram of an example, medical monitor with separate medication compartments, wherein the compartments and monitor communicate.

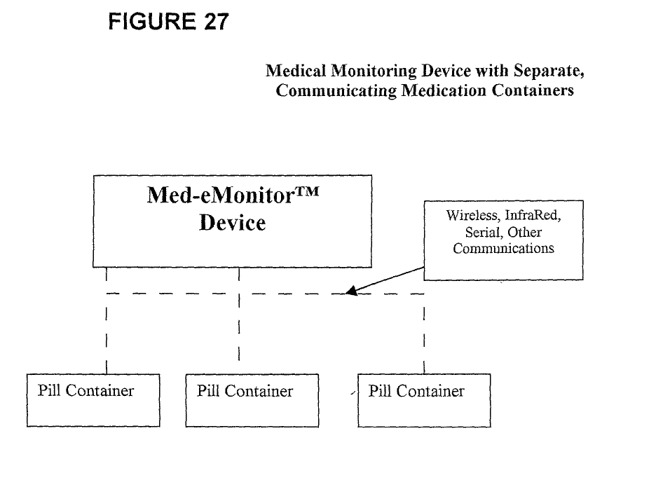

FIG. 28 is an example of medical icons useful in the medical monitor screen.

FIG. 29 is a block diagram of an example computer system useful for implementing the present invention.

### DETAILED DESCRIPTION OF THE INVENTION

### Table of Contents

I. Healthcare System Overview

II. Information Management System and Database

III. Enhanced Features of Medical Monitoring System

A. Mass Customization of Medical Protocols

B. Time-and-Event Driven Medical Treatment Plan

C. Risk-Stratified Triage and Medical Interventions

D. Self-Selected Synchronized Database-Linked Medical Monitors

E. Pharmaco-Economic Analysis

F. Creation and Self-Selection of Musical Alarms

G. Streaming Video and Pictorial Representation

H. Enhanced Validity in Pharmaceuticals and Drug Package Inserts

I. Mass Customizing Information Device Functions and Features

J. Dynamic, Mass Customizable, Interactive Screen and Voice

IV. Software and Hardware Embodiments

V. Conclusion

I. Healthcare System Overview

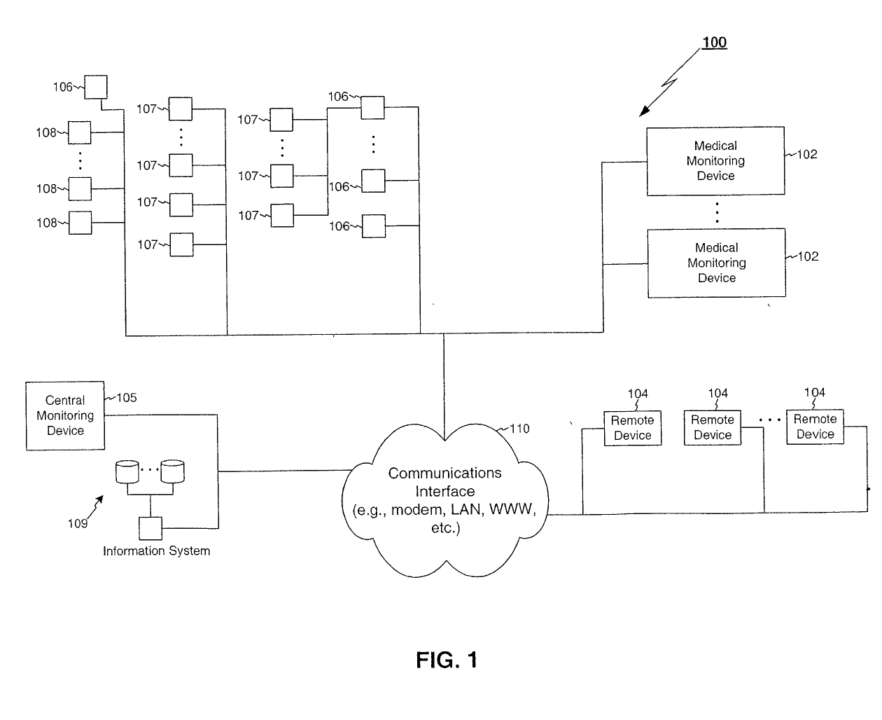

FIG. 1 illustrates, according to an embodiment of the present invention, an integrated healthcare system 100 for managing the treatment of one or more patients (not shown). Healthcare system 100 includes at least one medical monitoring device 102, remote device 104, central monitoring device 105, monitor 106, sensor 107, supplementation delivery device 108, and information management system 109. System 100 permits bidirectional communication among the system components through a diverse communications interface 110, which includes wired or wireless local area networks (LAN) or wide area networks (WAN), such as an organization's intranet, the global-based Internet (including the World Wide Web (WWW), or the like.

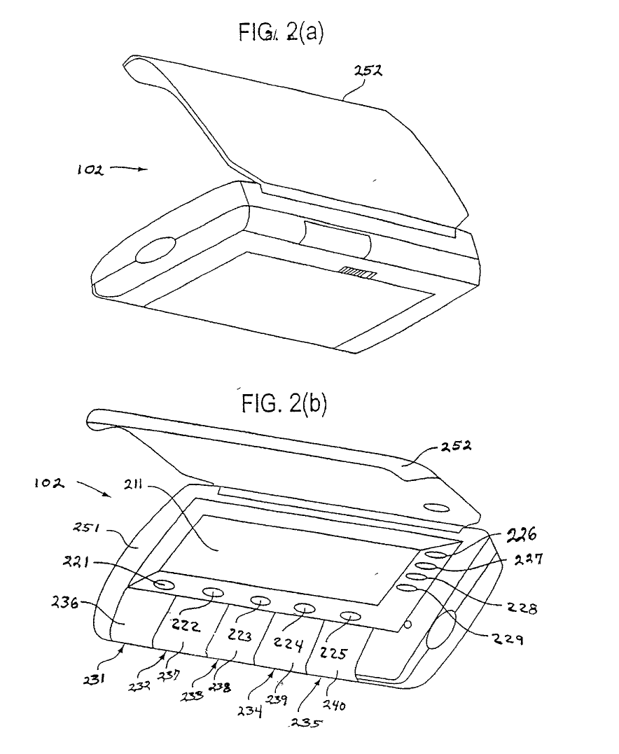

One component of system 100 is a medical monitoring device 102, which is preferably, but not necessarily, of the type described in commonly owned U.S. Pat. No. 6,102,855, (herein referred to as the '855 patent. FIGS. 2(a) and 2(b) illustrate an embodiment of a medical monitoring device 102 which has a top face 251 that includes openings for a LCD 211, dedicated keys 221-225, soft function keys 226-229 and medication compartments 231-235. Medication compartments 231-235 have respective compartment doors 236-240 covering them. Medical monitoring device 102 also has a hinged top cover 252 for opening and closing medical monitoring device 102. Medication compartments 231-235 are housed in a tray that inserts into the bottom of medical monitoring device 102. The tray may be of various sizes, and may even be large enough to maintain a pill bottle, a medication inhaler, syringe, or one or more other large medication containers. Further, medication compartments 231-235 can directly accommodate liquid, injectable, or aerosolized medications. Alternatively medical monitoring device 102 may lack medication compartments. The medication compartments may be separate from the monitoring device, any may communicate with the monitoring device via wireless, infrared, serial port or other means of communication as demonstrated in FIG. 31.

Compartment switches (not shown) signal a microcontroller (not shown) located with medical monitoring device 102, indicating when compartment doors 236-240 are opened and closed. Each of medication compartments 231-235 and its respective compartment door 236-240 is associated with a compartment switch (not shown) that senses the opening and closing of the compartment door. When a particular compartment door 236-240 is opened, the associated compartment switch signals the microcontroller, and the microcontroller processes the signal according to the specific status of medical monitoring device 102 at that time. For example, if a compartment door is opened during a scheduled medication alarm, the microcontroller interprets this as an indication that the patient has complied with the prescribed schedule for the particular medication contained in that compartment. The compartment switches thereby facilitate the ability of medical monitoring device 102 to track a patient's adherence to prescribed medication schedules.

Medical monitoring device 102 provides a means for interacting with the patient through a series of operations controlled by dedicated keys 221-225 and soft function keys 226-229. Dedicated keys 221-225 enable the patient to retrieve information concerning specific medications contained in medication compartments 231-235 or concerning specific diseases. Soft function keys 226-229 enable the patient to respond to patient queries concerning medication side effects, adherence to medication schedules, treatment progress, health status assessment and patient's quality of life. Soft function keys 226-229 further facilitate other operations, such as a programming mode for modifying device settings and adjusting medication schedules and treatment data, and other types of information capture. In an embodiment, a touch screen can be provided to function in the same manner as dedicated keys 221-225 and soft function keys 226-229.

Each of dedicated keys 221-225 is associated with the particular medication compartment located adjacent to it. Specifically, dedicated key 221 is associated with medication compartment 231, dedicated key 222 is associated with medication compartment 232, dedicated key 223 is associated with medication compartment 233, dedicated key 224 is associated with medication compartment 234, and dedicated key 225 is associated with medication compartment 235. Each dedicated key 221-225 controls the display or entry of information related to the specific medication stored in its associated medication compartment or related to the medical condition for which that medication is prescribed.

Medical monitoring device 102 collects and reports treatment information pertaining to a treatment regimen for one or more patents.

Treatment information includes: (1) response data; (2) physiologic data; (3) data related to cellular, molecular, and endocrine or metabolic mechanisms; (4) data concerning a patient's adherence to a treatment regimen; (5) data concerning the monitoring of medication compliance; and (6) any other data relating to any aspect of a patient's health or general quality of life, including genomic and proteomic data. Medical monitoring device 102 reports the treatment information to remote devices 104 or central monitoring device 105 for analysis, as described in further detail below. The contemporaneous exchange of treatment information promotes real-time or near term monitoring of the progress of the treatment regimen, and elements of a patient's health and physical status. The real-time or near term monitoring facilitates various tasks including, but not limited to, conducting a health status assessment, monitoring adverse or side effects experienced by the patient, monitoring treatment progress, monitoring physiologic data from serum or urine, monitoring events at the cellular, molecular and endocrine levels, assessing the patient's general quality of life, and automatically modifying of a treatment regimen.

When analyzed treatment information is received from remote devices 104 or central monitoring device 105, medical monitoring device 102 executes an interaction with the patient. The interaction comprises reporting to the patient one or a combination of the following: (1) educational information concerning patient health status, treatment progress, or other information relating to a disease(s) being treated or the treatment regimen being administered; (2) patient instructions (i.e., instructing the patient to modify medication dosage or schedule, instructing the patient to use a physiologic monitor, or (3) enter a physiologic value, or write down a physiologic value for later data entry, instructing the patient to schedule a visit, instructing the patient to go to the emergency room, or the like); or (4) patient, monitoring, query or other data relating to any aspect of the health or general quality of life of the patient.

If the analyzed treatment information comprises patient query data, once the patient query data is transmitted to medical monitoring device 102, the device either promptly executes a Patient Query mode to ask the patient to respond to a series of health-related questions, or stores the patient query data for subsequent execution of the Patient Query mode, depending on the status of an execution command. The execution command is either transmitted with the patient query data or stored in medical monitoring device 102. The execution command is set or established by a healthcare provider operating one of remote devices 104, or by medical monitoring device 102 itself through artificial intelligence means, and depends upon the urgency of receiving responses to the particular queries transmitted to medical monitoring device 102. The timing associated with the execution command is based on the disease(s) being treated and the treatment regimen being administered. After medical monitoring device 102 executes the Patient Query mode, the patient response data is either stored in medical monitoring device 102 for subsequent transmission, or promptly transmitted to the remote device 104 for storage and analysis as determined by the execution command.

System 100 also includes one or more monitors 106, sensors 107 and supplementation delivery devices 108, which are designed to communicate with one another and other system components via wired or wireless technologies. Collectively, monitors 106 and sensors 107 tracks or assesses one or a combination of physiologic, cellular, molecular, endocrine, metabolic or like health-related events, as determined by the particular requirements of the treatment regimen that is being administered. In particular, monitors 106 include, but are not limited to, devices that monitors a patient's medical treatment, medication usage, health status, and quality of life; devices that are external or implanted to monitor physiologic functions including, but not limited to, one or a combination of temperature, blood pressure, pulse, blood glucose level, EEG, EKG, pH and p02; and external or implanted devices that monitor hormone levels, ions, cellular mechanisms and molecular events.

Medical monitoring device 102, or one or more of monitors 106, also interface with one or more sensors 107 that are located externally to, or implanted within, the patient. Sensors 107 are responsible for monitoring different body processes. For example, one sensor 107 might be responsible for monitoring circulating peptide, molecular, ionic, and hormone levels in the blood, and additional levels, such as glucose, calcium, potassium, sodium, ketone, anemia, pH, p02 and the like. A second sensor 107 might be responsible for monitoring heart function. A third sensor 107 might monitor pulmonary (lung) function. A fourth sensor 107 might monitor blood pressure. Additional sensors 107 can be included to monitor additional body processes (such as, receptor levels, cell counts, or the like) as would be apparent to one skilled in the relevant art(s).

In an embodiment, monitors 106 and sensors 107 are externally or remotely located with respect to a medical monitoring device 102. However in another embodiment, monitors 106 and sensors 107 are located within or a part of a medical monitoring device 102 such that the medical monitoring device 102, itself, can track or assess health-related events.

System 100 also includes one or more supplementation delivery devices 108. Supplementation delivery devices 108 are externally or internally located to deliver appropriate types or quantities of pharmaceuticals, hormones, nutriceuticals, or other supplements. The type or quantity is based on the processes and levels tracked or assessed by monitors 106 and sensors 107.

In an embodiment, the treatment data collected by monitors 106 and sensors 107 are transmitted to medical monitoring device 102, where it is analyzed or stored for immediate or delayed transmission to other system components, such as remote devices 104 or central monitoring device 105 (both are described in further detail below). In another embodiment, the treatment data, collected by monitors 106 and sensors 107, is transmitted in real-time or near term directly to remote devices 104 or central monitoring device 105, for storage or analysis. In either embodiment, the timing or content of the transmission depends on the event(s) or treatment data monitored, information generated therefrom, the disease being treated, the treatment regimen being administered, or like criteria set or established by medical monitoring device 102, remote devices 104 or central monitoring device 105.

Medical monitoring device 102, remote devices 104 or central monitoring 105 also determines and controls the activation of designated monitors 106 or sensors 107 at particular times or during particular time intervals. One or more of these system components also controls the release of the supplements from supplementation delivery devices 108, based on an analysis of the collected treatment data. Moreover, one or more of these system components manages the communication among monitors 106, sensors 107, and supplementation delivery devices 108, including the timing of such communications, the nature and type of communication, and the type of information communicated.

Accordingly, one or more of medical monitoring devices 102, remote devices 104 and central monitoring device 105 are programmable, alone or in combination, to analyze treatment data collected by monitors 106 and sensors 107 in real time or on a periodically scheduled basis. These system components include software modules having diagnostic routines or subroutines that function like a physician conducting a clinical examination. The diagnostic modules provides instructions to determine when an event(s) should be monitored, which event(s) to monitor, how those event(s) should be monitored, and the particular monitor(s) 106 and monitoring sequence employed. Therefore, a particular health outcome(s) determine the timing and informational content of the transmissions.

In an embodiment, the diagnostic modules analyzes data collected in response to a patient query transmitted to medical monitoring device 102. The response data would be compared to target data that could have been transmitted with the query data, or target data previously stored in medical monitoring device 102. If the diagnostic modules reside in medical monitoring device 102 and the response data meet certain predefined criteria, medical monitoring device 102 would promptly transmit the response data to remote devices 104 or central monitoring device 105 for storage and further analysis. The predefined criteria comprise target data from which the response data should not deviate, a template with which the response data is compared, or any other methodologies or techniques for analyzing the response data in view of a prescribed baseline or range of values. In this fashion, the methods and systems of the present invention provides for the outcome of medical treatment to be monitored, analyzed, and compared to normative, targeted, or desired goals of treatment. Should any deviations from the norms or targets be detected, the methods and systems of the present invention provides for the modification of the patient monitoring as well as the treatment regimen, itself.

It would be apparent to one skilled in the relevant art(s) that a multitude of different modules can be developed to provide for prompt transmission of patient data should a single data element, sequence of data elements, or combination of data elements concerning a patient's health status be entered into or monitored by medical monitoring device 102, monitors 106 or sensor 107. Hence, medical monitoring device 102, remote devices 104 and central monitoring device 105 are programmable to continuously alter the selection, combination, timing, duration, sequencing, activation and polling of monitors 106 and sensors 107 to most effectively manage a patient's healthcare as required by the disease(s) being treated or treatment regimen being administered.

As described, medical monitoring devices 102, remote devices 104 and central monitoring device 105 can poll or receive updates from monitors 106 and sensors 107 to collect treatment information. Remote devices 104 and central monitoring device 105 are also capable of polling or receiving updates from medical monitoring devices 102 to retrieve treatment information on a continuous, periodic, spontaneous, or random basis. In an embodiment, remote devices 104, central monitoring device 105 and medical monitoring devices 102, as appropriate, analyze previously captured treatment information to determine the frequency of the polling or updates, and whether to initiate continuous, periodic, spontaneous, or random monitoring. Moreover, in cases where the patient's treatment regimen includes an “unforgiving” medication that must be taken according to precise schedules and quantities to avoid dire clinical problems, the polling frequency can be adjusted to remotely, yet closely, monitor health status data and compliance with treatment regimens.

As stated, system 100 also includes one or more remote devices 104, central monitoring device 105 and information management system 109. Each of these system components can represent one or more computers providing various shared resources with each other and to other system components (e.g., medical monitoring device 102, monitor 106, sensor 107, and supplementation delivery device 108). The shared resources include files for programs, web pages, databases and libraries; output devices, such as, printers, plotters and audio/video recorders and players; and communications devices, such as modems or Internet access facilities. The communications devices can support wired or wireless communications over communications interface 110, including satellite, terrestrial (fiber optic, copper, coaxial and the like), radio, microwave, infrared, laser and any other form or method of transmission.

Remote devices 104, central monitoring device 105 and information management system 109 are configured to support the standard Internet Protocol (IP) developed to govern communications over public and private Internet backbones. The protocol is defined in Internet Standard (STD) 5, Request for Comments (RFC) 791 (Internet Architecture Board). Remote devices 104, central monitoring device 105 and information management system 109 can also support transport protocols, such as, Transmission Control Protocol (TCP), User Datagram Protocol (UDP) and Real Time Transport Protocol (RTP). In an embodiment, remote device 104, central monitoring device 105 and information management system 109 use a TCP/IP protocol to provide communications between any two nodes in system 100. These system components are also configured to support various operating systems, such as, the Netware™ system available from Novell®; the MS-DOS®, Windows NT® or Windows®3.xx/95/98/2000 system available from Microsoft®; the Linux® system available from Linux Online Inc.; the Solaris™ system available from Sun Microsystems, Inc.; or the like, as would be apparent to one skilled in the relevant art(s).

Remote devices 104 are portable, hand-held or desktop devices (such as, a personal computer, personal digital assistant (PDA), telephone, pager, television, and the like) that provide real-time or near term treatment information to one or more users, including healthcare providers, family members, caregivers (i.e., sitters, nannies, au-pairs, etc.), system analyst, administrators, or operators, and the like. Each of these individuals would have access to at least one remote device 104. The healthcare providers can include a team of professionals, such as, one or more physicians, case managers, nurses, physical therapists, personal emergency response personnel, psychiatrists, pharmacists, physician assistants, and the like. Since medical monitoring devices 102, monitors 106, and sensors 107 assesses or tracks different aspects of a patient's condition, it may become necessary that one or more of the team members be promptly notified about emerging aspects of the patient's physical state. Then, according to an analysis of treatment information concerning the patient's physical state, medical monitoring device 102 or one of the other system components (such as central monitoring device 105 or a remote device 104) determines which team member(s) (via, e.g., remote device 104) to notify, the timing of such notification, and the appropriate data to convey; and transmits the data accordingly.

Additionally, the diagnostic software modules operating on remote devices 104, medical monitoring device 102 and central monitoring device 105 can be altered or reprogrammed by a healthcare provider or a system operator. In an embodiment, the diagnostic modules are programmable to automatically analyze the treatment information transmitted to remote device 104 or central monitoring device 105 if the designated healthcare provider is not available to respond to the transmitted information within a specified time frame. Since the transmitted treatment information potentially represents an aspect of the patient's condition that could mandate critical and prompt or immediate review, the diagnostic modules enable prompt action to be taken to examine the patient, further query the patient, change the patient's treatment regimen, instruct the patient to go to the emergency room, or the like. The diagnostic modules are also programmable to promptly transmit a message to another portable, remote device 104 or similar portable device (not shown), that is being carried by the healthcare provider or to the healthcare provider's answering service. As such, system 100 provides for a patient and healthcare provider to be thereby linked, via a wired or wireless means, to a series of two or more devices (e.g., medical monitoring device 102, remote devices 104 and central monitoring device 105), each of which capable of analyzing information and determining the appropriate timing and content of information communicated to the healthcare provider concerning the patient's health status.

For example, with regard to an outpatient with a congestive heart failure condition, when medical monitoring device 102 detects an increase in the patient's blood pressure of at least 30 mm mercury, system 100 (e.g., medical monitoring device 102, a remote device 104 or central monitoring device 105) would operate to notify the patient's case manager of the developing circumstances. Further, if the increase in blood pressure is accompanied by chest pain, system 100 would notify the patient's case manager and visiting nurse. Going one step further, if certain cardiac enzyme elevations, muscle receptor alterations, or other molecular changes are occurring, system 100 would notify the case manager, visiting nurse, and cardiologist. Finally, if the patient also increases nitroglycerin tablet intake, system 100 would notify a nearby emergency response center and request the dispatch of an ambulance, in addition to notifying the above mentioned healthcare providers.

Central monitoring device 105 is another component of system 100 that polls, or receives updates from, the other system components, namely medical monitoring devices 102, remote device 104, sensors 107 and monitors 106 to obtain treatment information. Hence, central monitoring system 105 functions as a command and control center to govern and regulate all of the monitoring processes and delivery of supplements, compare the monitored processes and levels to normative values, effectuate a series of treatments to restore homeostasis, perform additional diagnostic tests, issue alerts to healthcare providers (via, for example, remote devices 104), and administer other remedial functions. Central monitoring device 105 also performs diagnostics on the other system components, such as medical monitoring devices 102, monitors 106, sensors 107, and supplementation delivery devices 108. If a malfunction is detected, central monitoring device 105 analyzes the malfunction to correct it, notify the appropriate system administrator or healthcare provider (via, e.g., a remote device 104), or both.

For example, a patient could be attached to a glucose monitor 106 that monitors glucose levels in the blood and reports them to central monitoring device 105 on demand or according to periodically scheduled rates. Upon receiving the data representing glucose levels, central monitoring device 105 compares the levels to normative values. When the glucose levels are within the range of normative values, central monitoring device 105 determines that no treatment is necessary. When the reported glucose levels are outside the range of normative values, central monitoring device 105 instructs the patient (via, e.g., medical monitoring device 102) to administer an injection with additional insulin, or eat a piece of fruit or candy bar, depending upon whether the glucose levels are too high or too low, respectively. Alternatively, central monitoring device 105 could activate an implanted insulin pump (i.e., supplementation device 109) to release insulin into the patient's bloodstream, or contact a healthcare professional (via, e.g., a remote device 104) that the patient is in danger.

The specific course of action taken by system 100 in response to a patient's condition is prescribed or can be modified by a designated healthcare provider. The appropriate analytical device (e.g., medical monitoring devices 102, remote devices 104 and central monitoring device 105) would execute an analysis (i.e., decision tree analysis) that results in different actions depending on the monitored conditions or events. For example, supplementation delivery device 108 could be activated to administer a drug to block, for example, a heart attack. If an ambulance service is notified, the diagnostic modules of system 100 could continue to monitor data from, for example, monitors 106 until the ambulance arrives, as well as transmit the data to healthcare providers, administer additional drugs via supplementation delivery device 108, and titrate the dosing of the drugs based upon monitored events that relate to blood levels of the drug, degree of heart muscle damage, degree of chest pain experienced by the patient, and the like.

Besides monitoring heart failures and diabetes as described above, system 100 is configurable to monitor and treat other anticipated medical conditions, such as, renal or kidney disease, liver disease, hypertension, endocrine imbalance, cancer, lung and heart disease, viral, bacterial and fungal diseases, and antigens associated with specific human pathogens (i.e., if the patient is receiving antibiotics, a healthcare provider would monitor whether the organism becomes resistant to the antibiotics or increases in number) and the like.

Central monitoring device 104 and the other system components also exchanges communications with information management system 109. In an embodiment, an Open DataBase Connectivity (ODBC) or Java DataBase Connectivity (JDBC) protocol permits central monitoring device 105 and the other system components to exchange wired or wireless transmissions with information management system 109 over communications interface 110. Information management system 109 includes one or a combination of resident or third-party databases, inference engines, neural network systems, knowledge management systems, profiling engines, concept engines, and other information searching, processing or reasoning systems. In an embodiment, information management system 109 applies pattern-matching methodologies (based on, for example, Bayesian, Shannon, or similar inference theories) to perform diagnostics or extract key concepts from treatment information collected by the other system components. The key concepts are used to identify symptoms, predict behavioral patterns, recommend treatments, determine prognosis, or the like. In an embodiment however, a healthcare provider (using remote device 104) performs the diagnostics independently, or with the benefit, of data generated by information management system 109.

Information management system 109 comprises a collection of integrated records used to support healthcare system 100. In an embodiment, information management system 109 includes a relational or object oriented (OO)/component based database management system (not shown) that controls the storing, retrieving and updating of data and meta-data in the records of information management system 109. The database management system also controls data integration, enforces integrity rules and constraints (including data integrity and referential integrity), and enforces security constraints.

In an embodiment, a Secure Sockets Layer (SSL) provides for a secure data path for all communications among the system components over communications interface 110. Additionally, central monitoring device 105 and information management system 109 functions to maintain the security, integrity, and proprietary of all system information, in particularly to user access, data, and communications of patient records residing in the databases of information management system 109. User authentication (e.g., username/password; fingerprint, retina, facial, or voice identification; or the like) determines access, roles, security level, and functions with respect to all system components, namely central monitoring device 105 and information management system 109. For example, user roles are configured to permit designated healthcare providers to review a patient's treatment information or alter the patient's treatment regimen. In other embodiments, access to the resources of system 100 is controlled by firewalls, encryption, or like security techniques.

In short, the databases and other information searching, processing and reasoning systems of information systems 109 are operable to: (1) better analyze the inputted treatment information from the peripheral sensors 107 and monitors 106; (2) download appropriate information from other databases or search engines of information management system 109 and communicate that information to the patient (via, e.g., medical monitoring device 102); (3) apply database information to deploy and control other medical monitoring devices 102, monitors 106, and sensors 107; and (4) apply database information to deliver medical treatment and medication (via, e.g., supplementation delivery devices 108).

For example, a glucose monitor 106, attached to a patient, could wirelessly transmits treatment information indicating the patient's glucose level to the patient's medical monitoring device 102 or central monitoring device 105 at selected time intervals. Medical monitoring device 102 or central monitoring device 105 could also periodically poll an insulin administration monitor 106 to receive data indicating the date, time, and dosing amount of the patient's insulin medication. After receiving the treatment information, medical monitoring device 102 or central monitoring device 105 could query a diabetes disease management database within information management system 109 to access and/or download a software module that, when applied to analyze the glucose data levels and insulin dosing levels, produces new instructions for the patient about insulin dosing and glucose monitoring. Furthermore, medical monitoring device 102 or central monitoring device 105 could query a nutrition database within information management system 109 to download new dietary instructions, and communicate the new insulin instructions and dietary instructions to the patient.

If, for example, patient monitoring reveals a glucose level that is dangerously high, posing a risk for the development of a hyperglycemic coma, medical monitoring device 102 or central monitoring device 105 could poll the diabetes disease management database of information management system 109, and access a software module to determine the need for cardiovascular monitoring. Medical monitoring device 102 or central monitoring device 105 could then initiate blood pressure and cardiac status monitoring. When medical monitoring device 102 or central monitoring device 105 receives treatment information concerning the patient's cardiac status, the diabetes disease management database could, once again, be accessed to determine whether, or the amount by which, to increase the next insulin dose. Medical monitoring device 102 or central monitoring device 105 would communicate the new dosing instructions to the patient.

Alternatively, the peripheral monitors 106, sensors 107, and supplementation delivery devices 108 are preferably equipped with the ability to access the databases of information management system 109, themselves, and directly communicate among one another, thereby bypassing medical monitoring device 102 or central monitoring device 105. This configuration provides a backup system, should medical monitoring device 102, central monitoring device 105 or both fail, or should such an alternative be more desirable based on cost, portability, efficiency, or other factors.

The methods and systems of the present invention provide real-time or near-term patient monitoring and management of health status independent of, or in conjunction with, a healthcare provider by linking a variety of patient monitors 106 and sensors 107 with one another, and with various databases and search engines of information management system 109; providing a series of processing and reasoning capabilities to analyze monitored data, combined with information from the databases and search engines of information management system 109; and initiating various treatment functions (i.e., communicating treatment information to the patient or administering doses through attached supplementation delivery devices 108) based on the results of the data analysis. The methods and systems of the present invention also take advantage of continuously updated databases and knowledge management systems, that can be used to guide treatment of the patient's condition in real-time, to optimize the monitoring and management of a disease.

II. Information Management System and Patient Database

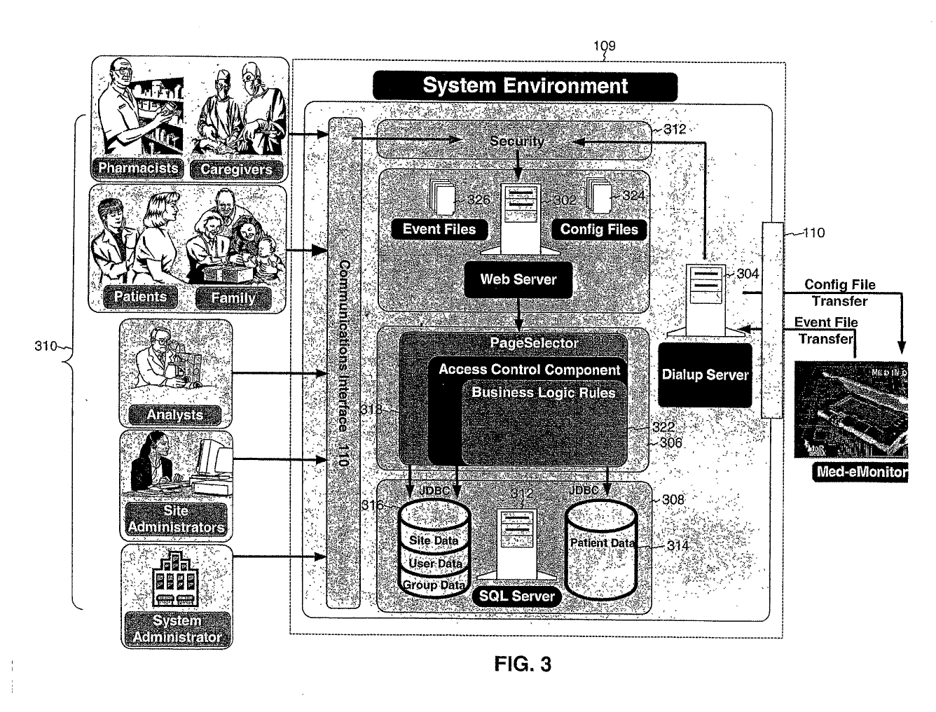

As described in reference to FIG. 1, information management system 109 includes one or more databases and other information searching, processing and reasoning systems. FIG. 3 shows an exemplary interaction of information management system 109 with other components of healthcare system 100. Information management system 109 includes a web sever 302 and dial-up server 304 that grant access to other components. Web server 302 manages on-demand data exchanges over communications interface 110 with the system users 310, including, for example, healthcare providers, family members, other caregivers, and the patients, themselves, (via, e.g., remote devices 104 or medical monitoring devices 102). Dial-up server 304, however, manages all scheduled communications over communications interface 110 with the other system components, including medical monitoring devices 102, monitors 106, sensors 107, supplementation devices 108, remote devices 104, and central monitoring device 105. For example, dial-up server 304 facilitates the polling or receipt of automatic transmissions of treatment information or medical regimens from medical monitoring devices 102.

Information management system 109 includes a security manager 312 that authenticates all users 310 and validates all requests by users 310. In an embodiment, security manager 312 employs role-based assignments to restrict the system contents to authorized users 310. Security manager 312 queries the records of Patient Data 314 (described below) and Site-User-Group Data 316 (described below) to assess the roles and validate queries based on the established profiles of the requesting user 310.

Another component of information management system 109 is database manager 306 that uses logic rules and similar inference methodologies to query the contents of information management system 109 or external databases or search engines. Database manager 306 includes a page selector 318, access controller 320, and database processor 322. Page selector 318 is programmable to format and configure data that is prepared and transmitted to the other system components. Access controller 320 queries the appropriate database for relevant records to authenticate users 310 and support other security protocols prescribed by security manager 312. Database processor 322 provides the business logic rules and similar inference methodologies that supports the searching, data mining, diagnostic reasoning and similar functions of information system 109.

One of the databases of information management system 109 is patient database 308 which is configurable for mass customization to convert a medical regimen into a series of real-time, time-and-event-driven communications delivered over time and space to remotely located medical monitoring devices 102; and operable to perform data analysis and produce reports to system users 310. System users may access these reports over remote devices 104 shown in FIG. 1. Database manager 306 queries patient database 308 over a JDBC protocol. In an embodiment, patient database 308 is an object-oriented database (such as a Microsoft® Access database system available from Microsoft Corporation) that is affiliated with a relational data warehousing environment (such as the Microsoft® SQL Server 7 database available from Microsoft Corporation). However, other types of databases (such as, a Microsoft® Excel database system available from Microsoft Corporation, a Paradox® database system available from Corel Corporation, a dBase® database system available from dBase, Inc., an Oracle 8i™ database system or Oracle Warehouse Builder database system available from Oracle Corporation, and the like) or querying protocols (such as, Basic, C, C++, and the like) can be used.

The records of patient database 308 are located in Patient Data 314 and Site-User-Group Data 316. Patient Data 314 contains a collection of all personal identification, demographics, physiological data and other information related to the patients that are being monitored or treated by healthcare system 100. The “User” records of Site-User-Group Data 316 include data pertaining to the individual users 310 (excluding the patients). The “Site” records include data pertaining to a clinic, hospital, research facility, sponsor or other institution or organization, where a group of users 310 are members or employees. The “Group” records includes data pertaining to a combination of patients (from Patient Data 314) that are assigned to a specific group for collective monitoring or analysis. As would be apparent to one skilled in the relevant art(s), Patient Data 314 and Site-User-Group Data 316 can be maintained in a common host database or in separate host databases.

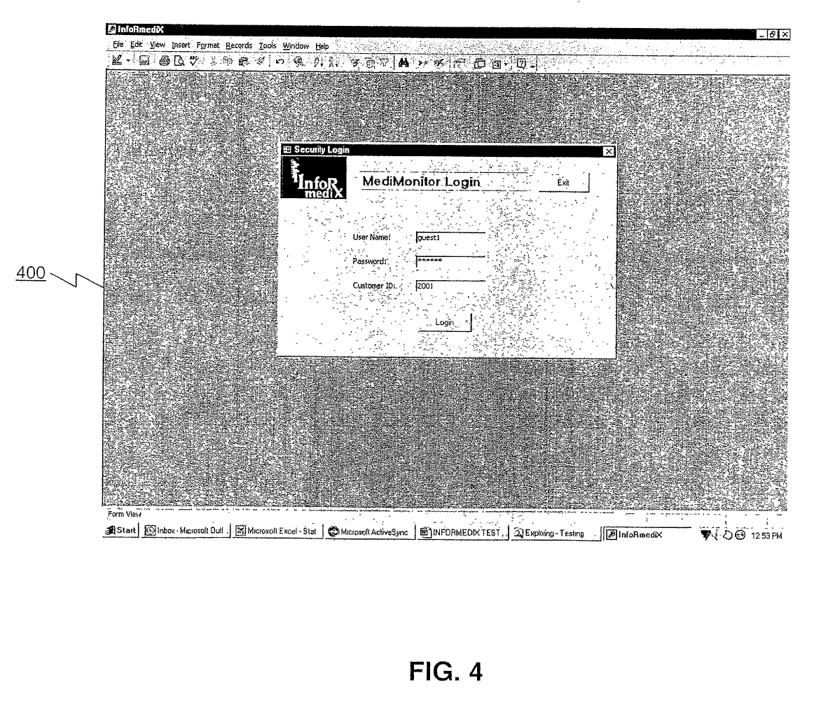

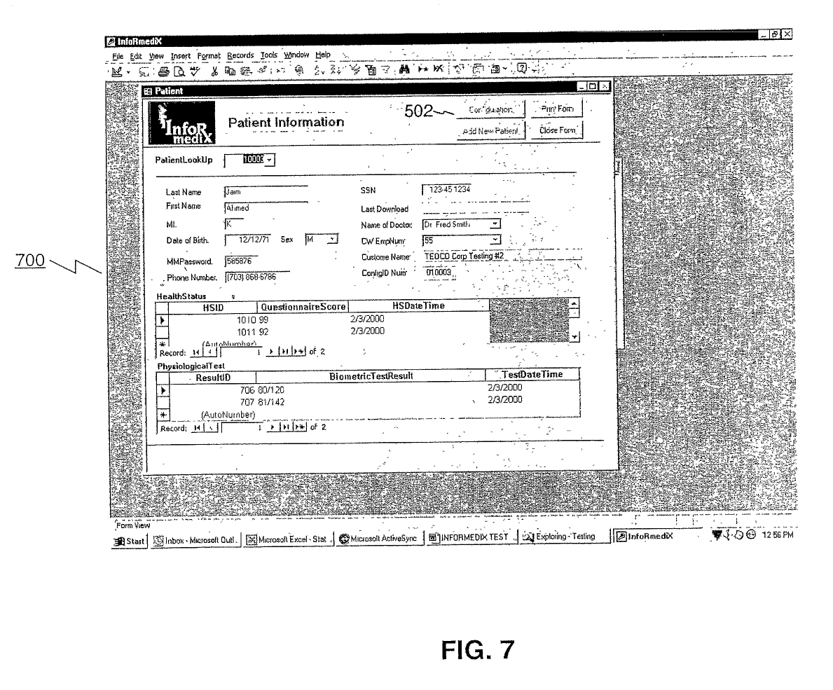

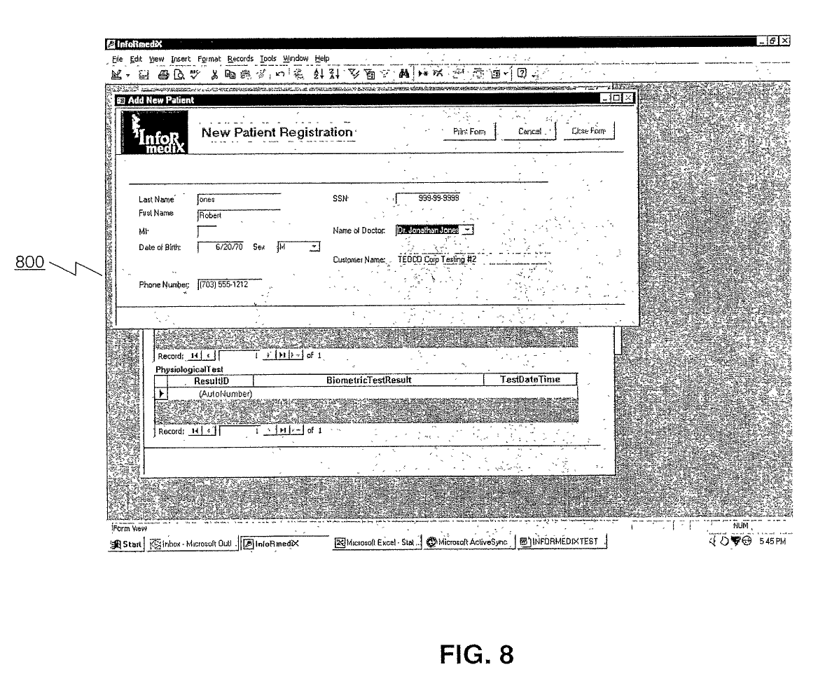

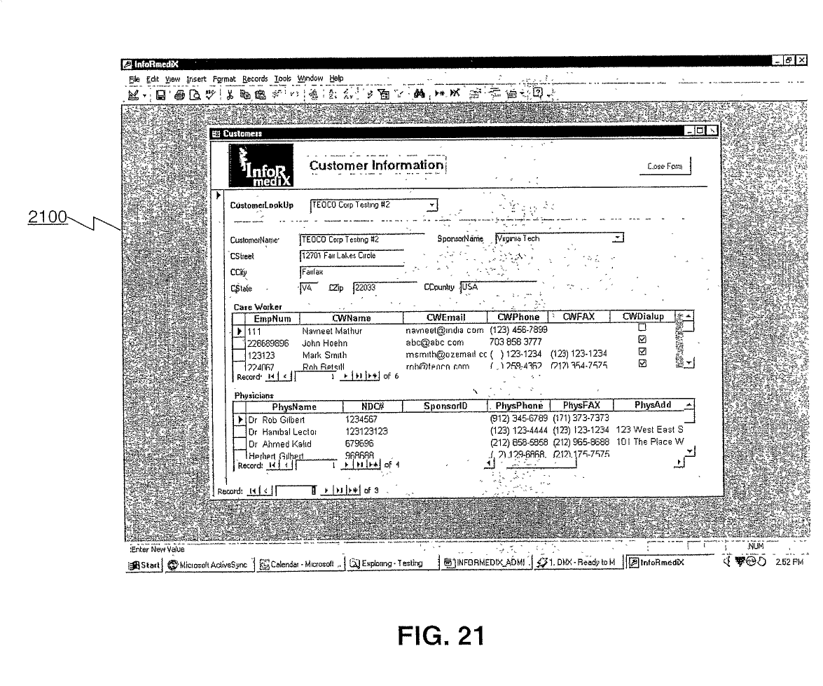

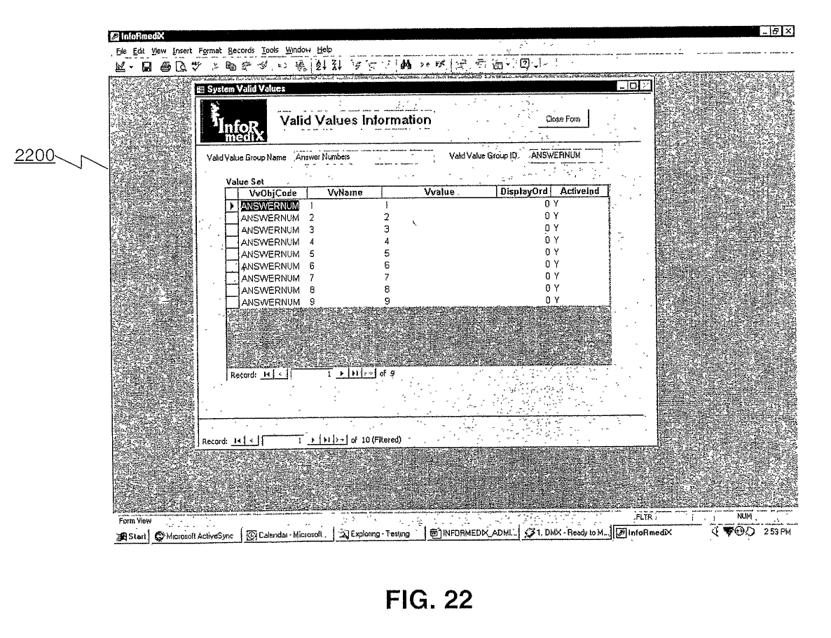

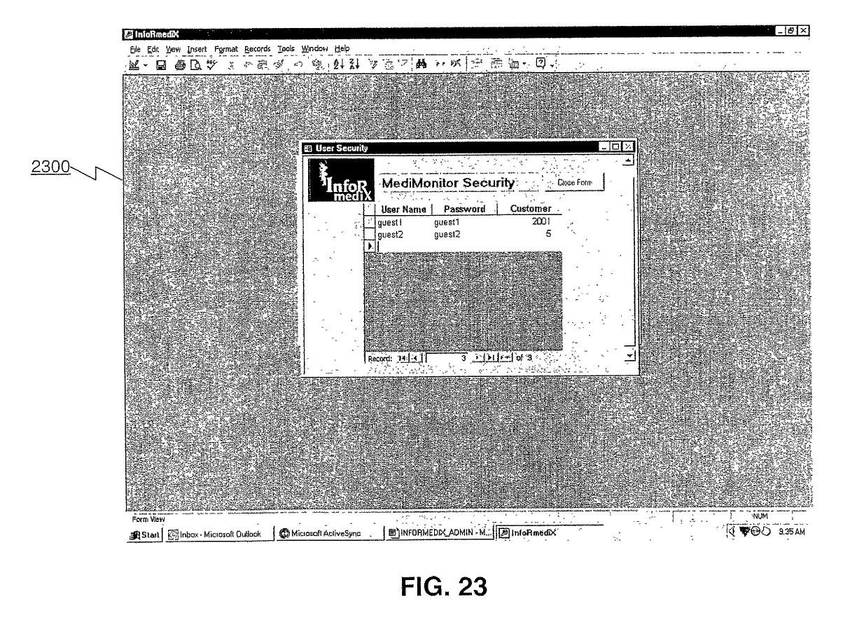

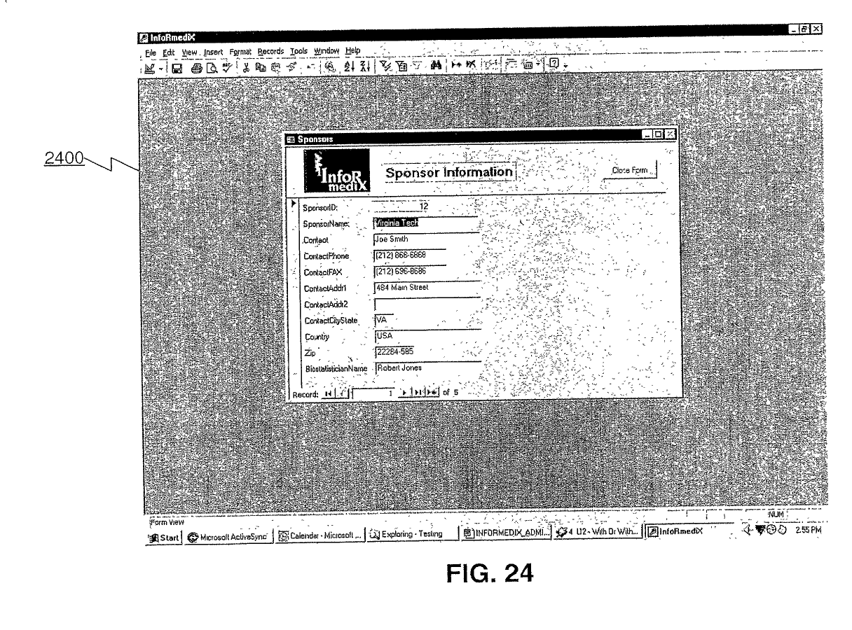

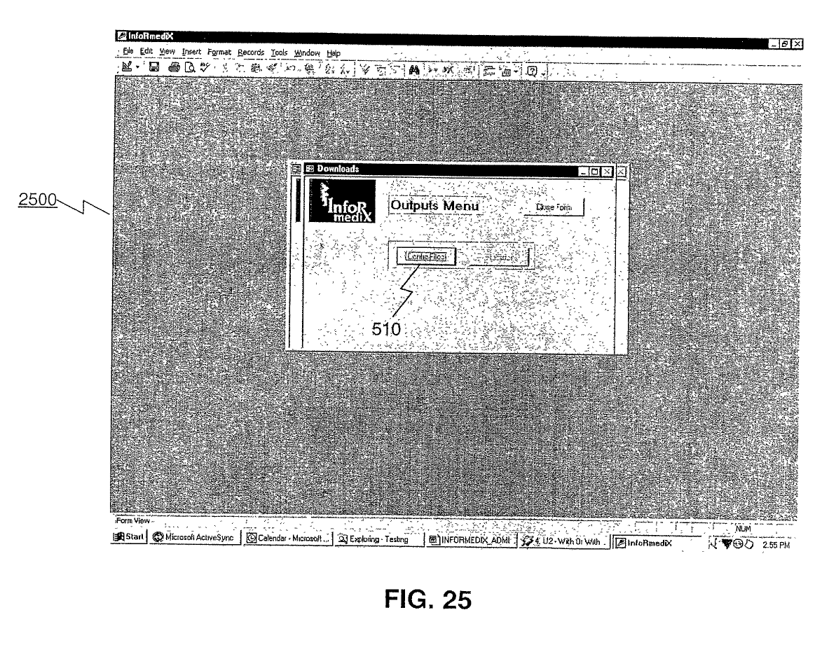

FIGS. 4-25 illustrate various text or graphical user interfaces depicting the organization of patient database 308 according to an embodiment of the present invention. In FIG. 4, graphical user interface (GUI) 400 shows that access to patient database 308 is password protected. As discussed in reference to FIG. 1 and FIG. 3, other methods of user verification can be used, including, but not limited to, fingerprint, retina, facial, voice identification, and like security measures enforced by security manager 312.

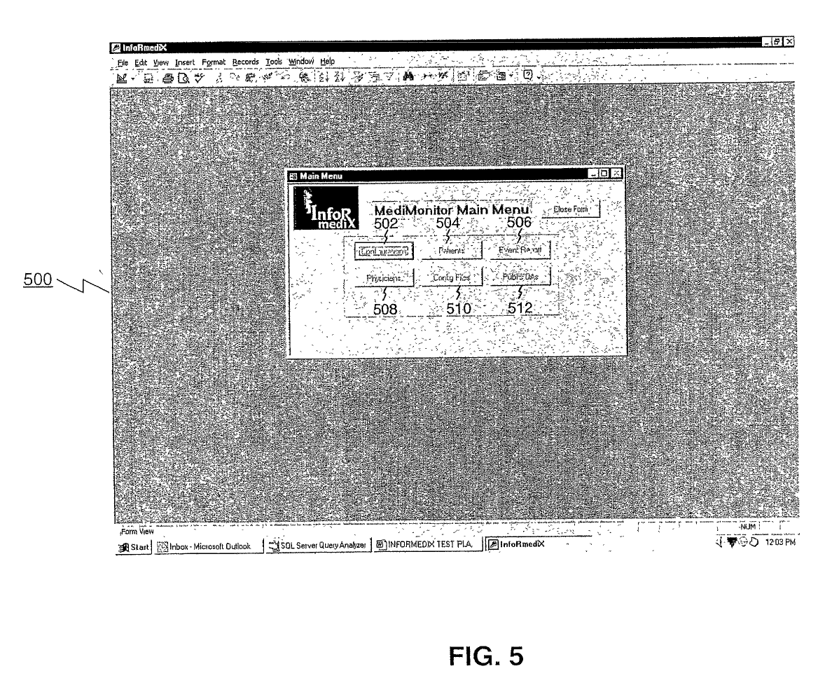

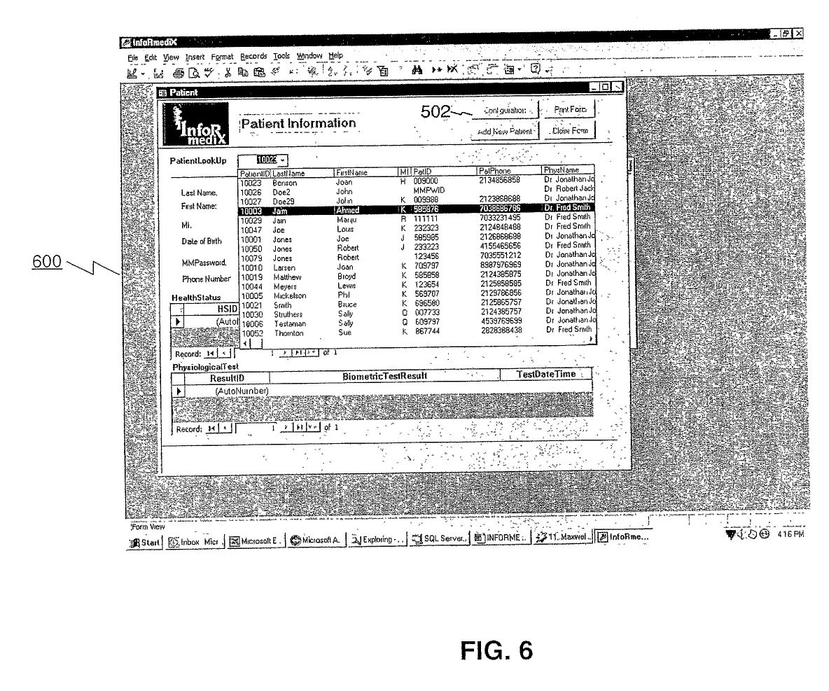

FIG. 5 illustrates a Main Menu GUI 500 according to an embodiment of the present invention. Main Menu GUI 500 contains six icons, or buttons, with each icon representing at least one object located within patient database 308. The icons include Configuration 502, Patients 504, Event Report 506, Physicians 508, Configuration Files 510, and Public QAs 512. Patients 504 provides access to the integrated records of Patient Data 314. As shown in FIGS. 6-8, the patient records include personal identification, demographics, physiological data, health status, other treatment information, and the like, pertaining to the patients being monitored or treated by healthcare system 100.

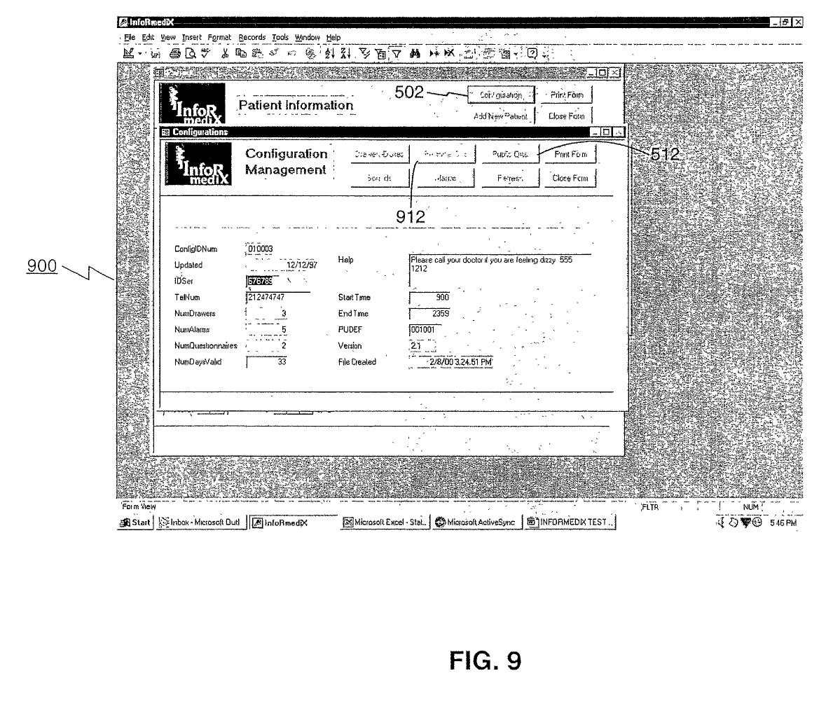

Configuration 502 provides access to data related to the patient's medical treatment plan, including patient medication, dosage, questionnaires, messages, and system configuration properties for a medical monitoring device 102 affiliated with a patient that will deliver the treatment plan to the patients in an automated sequential fashions. The records linked to Configuration 502 can be accessed directly from Main Menu GUI 500 as shown in FIG. 5, or after activating the icon for Patients 504 as shown in FIG. 6 and FIG. 9.

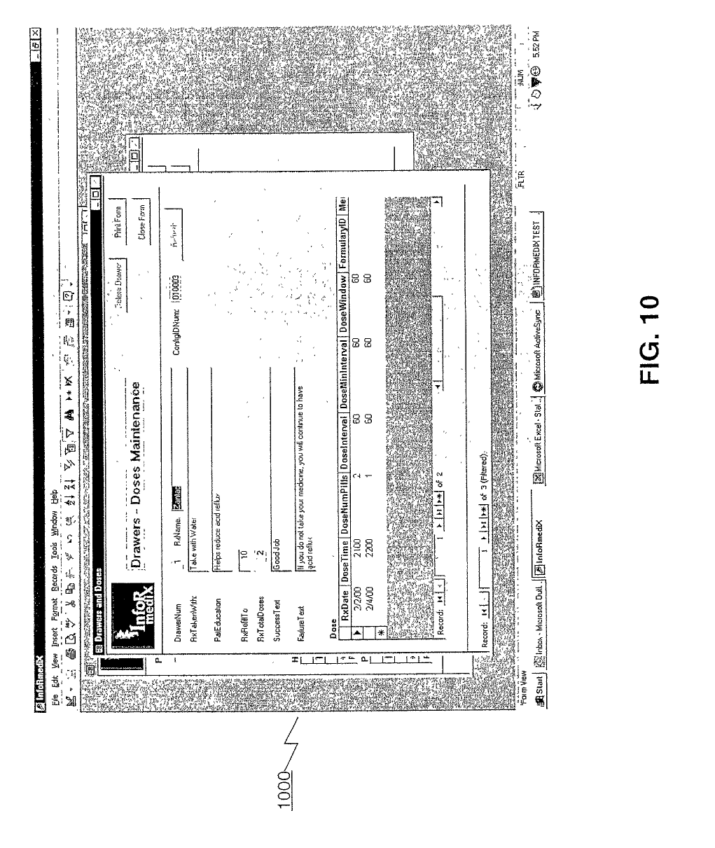

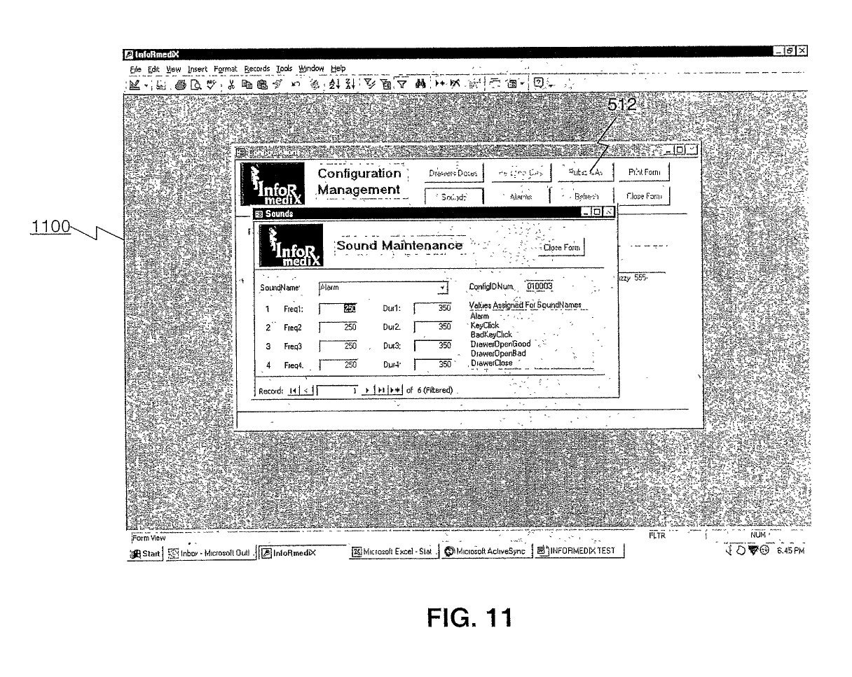

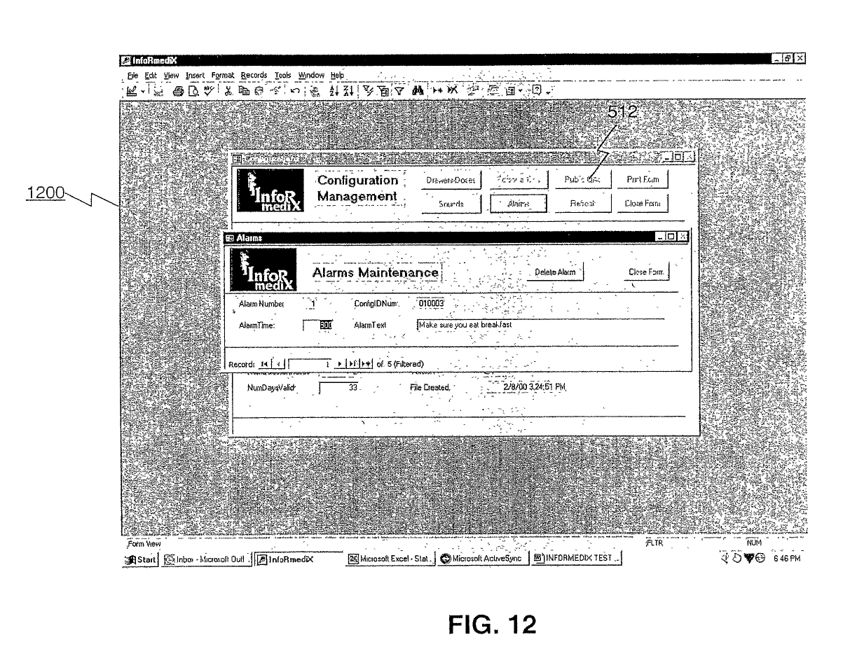

Upon activation, Configuration 502 is operable to prescribe a medical regimen for an individual patient or mass customization for a group of patients. As shown in FIG. 10, a submenu linked to Configuration 502 enables a healthcare provider to prescribe medication doses or assign medication compartments 231-235 for a medical monitoring device 102, as well as send instructions, success messages, failure messages or additional data related to the medication administration. Activating Configuration 502 also permits users 310 to configure sounds and alarms, shown in FIG. 11 and FIG. 12, respectively, for the medical monitoring devices 102. Each alarm has associated with it instructions and/or medical content that comprise elements of the patient's treatment plan.

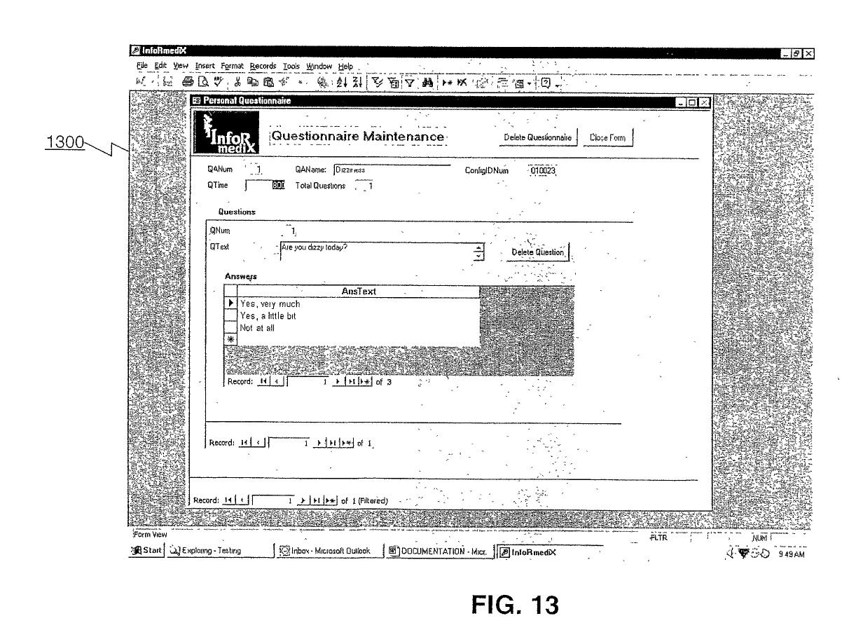

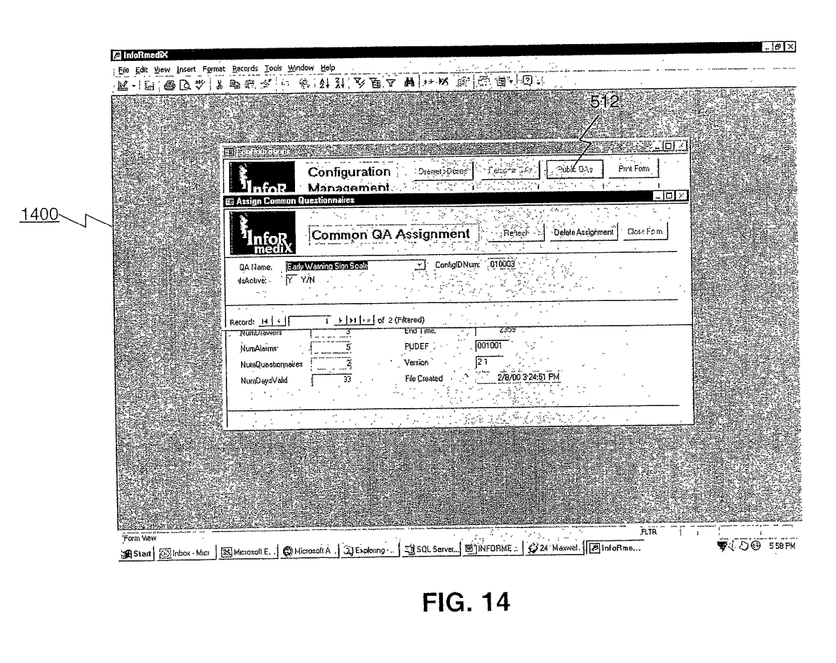

Referring back to FIG. 9, questionnaires can be created or modified through submenus linked to Configuration 502. User 310, such as a healthcare provider, can activate Personal QAs 912 to provide personal questionnaires regarding the status of a specific patient, or activate Public QAs 512 to provide public questionnaires related to a generic medical regimen for a group of patients. Personal and public questionnaires can be created by entering questions and possible answers as shown in FIG. 13. Public questionnaires can also be prepared by activating Public QAs 512 from Main Menu GUI 500, shown in FIG. 5. An example of a public questionnaire is shown in FIG. 13 in the Help field. FIG. 14 illustrates GUI 1400 that permits a public questionnaire to be assigned to a specific configuration.

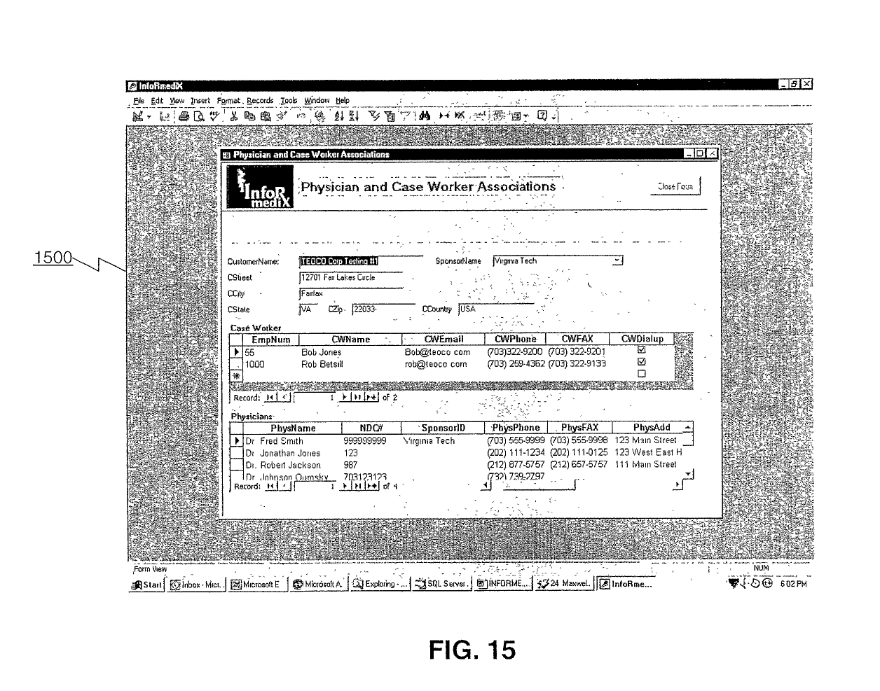

Referring back to FIG. 5, Physicians 508 provides access to Site-User-Group Data 316 that contains data pertaining to the healthcare providers or other users 310 affiliated with the patients. In FIG. 15, GUI 1500 provides an example of the type of personal identification, demographics, and the like data recorded for users 310.

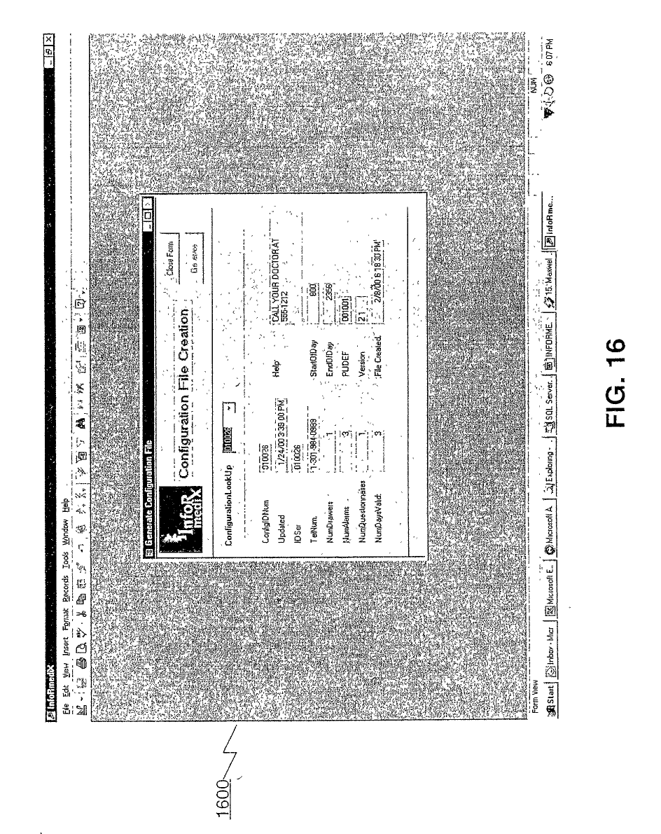

Main Menu 500 also includes an icon for Configuration Files 510. Activation of Configuration File 510 leads to GUI 1600 shown in FIG. 16. GUI 1600 facilitates the generation of one or more configuration files 324 used to transmit medical regimens to medical monitoring devices 102. In an embodiment, the records and files of patient database 308 are translated into software routines that convert the medical regimens into a series of prompt and record events. In this way, the patients get the benefit of both population-based as well as individualized assessment and/or treatment protocols on a real-time basis. Once the appropriate healthcare provider (i.e., user 310) populates the data fields in patient database 308 by selecting a combination of data elements for a medical treatment plan from an infinite number of possible data elements, the healthcare provider enters the data elements into fields of patient database 308. Patient database 308 converts these fields into configuration files 324 that are downloaded into the memories of the patient devices or monitors (namely, medical monitoring device 102, monitors 106, sensors 107 and supplementation devices 108). Alternatively, patient database 308 sequentially transmits the real-time prompt-and-record events to the patient devices. As a result, the patient management protocols can be instantaneously updated.

Patient database 308 enables alarm-based medication events, educational content messages, alarm-based treatment instructions, questionnaires, and any other elements contained in the medical regimens to be assigned to specific groups of patients or individual patients to be delivered over time and space to the remote monitoring devices. The events that are then prompted and recorded can be organized and managed as event files 326, which are collected by patient database 308 in an event log that can be accessed by activating Event Report 506 (see FIG. 3 and FIG. 5).

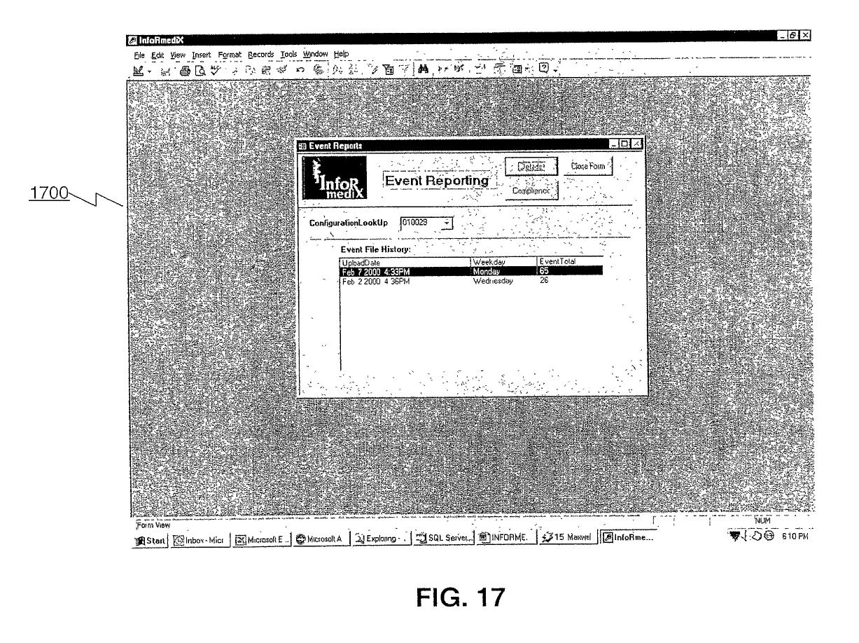

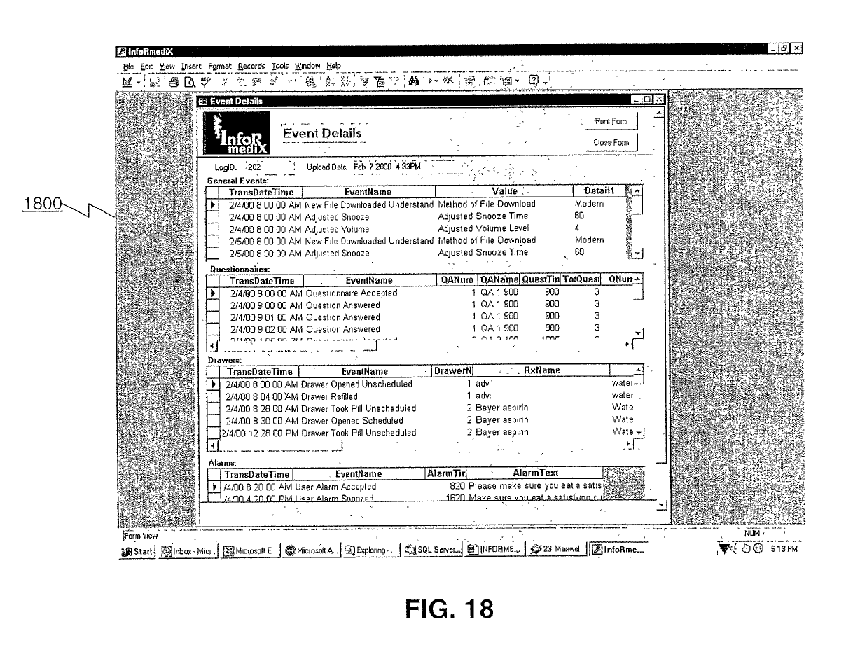

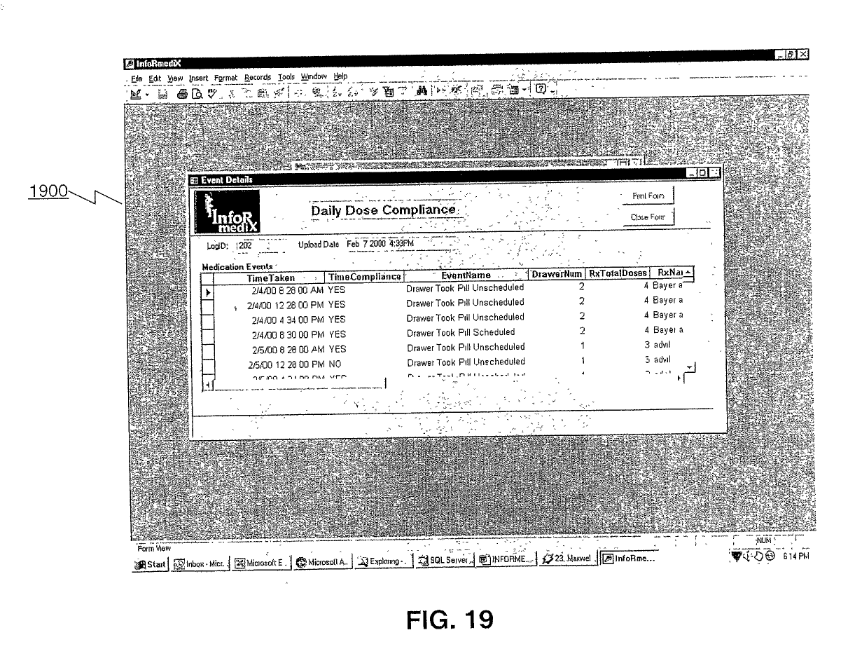

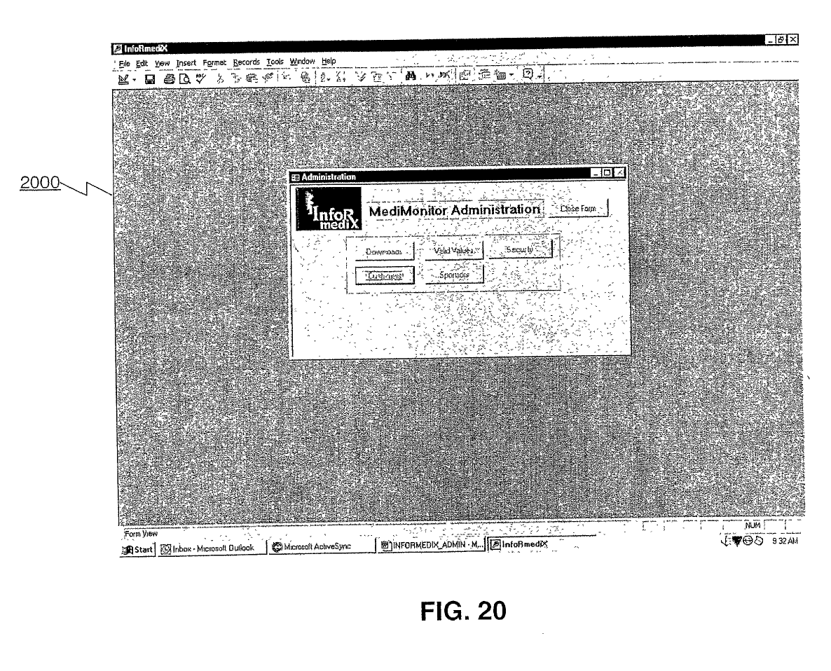

In FIG. 17, Event Report GUI 1700 illustrates an example of an event file history from all patient interactions with medical monitoring devices 102, monitors 106, sensors 107 and supplementation delivery devices 108. From GUI 1700, users 310 can activate an icon to gain a detailed listing of the events (e.g., drawer openings, alarms, questionnaire transmissions, and the like) as shown in FIG. 18, or a detailed listing of medication compliance (e.g., which may be reported as compliance ratios, compliance ranking, questionnaire responses, and the like) as shown in FIG. 19. FIGS. 20-25 show additional graphical user interfaces that are operable for organizing and managing patient database 308.

III. Enhanced Features of Medical Monitoring System

A. Mass Customization of Medical Protocols

In an embodiment of the present invention, system 100 is configurable to remotely modify medical protocols by subgroups preferably, but not necessarily, as described in commonly assigned U.S. patent Ser. No. 60/200,583, filed Apr. 28, 2000, (herein referred to as the '583 application).

The disclosure of the '583 application is incorporated herein by reference as though set forth in its entirety.

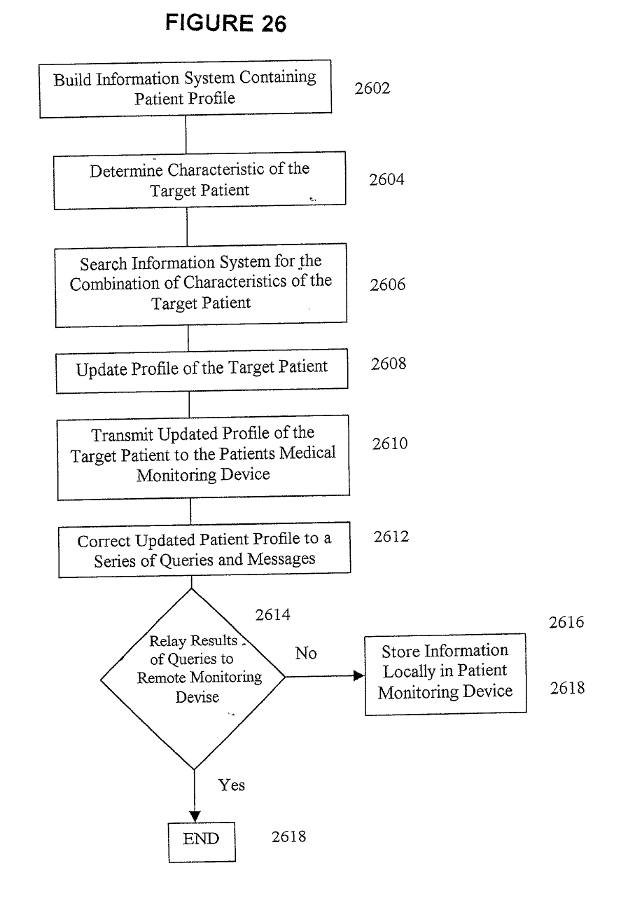

Referring to FIG. 26, a flowchart 2600 representing the general operational flow, according to an embodiment of the present invention, is shown. More specifically, flowchart 2600 depicts an example control flow involved in populating a profile fosr a new patient or updating the profile of an existing patient.

The present invention describes methods and apparatus useful in remotely modifying medical protocols by subgroups that are defined by specific database characteristics. In addition individual patient protocols may also be remotely modified. The method has application in medical databases containing the data collected by remote device monitoring of outpatients. These outpatients may be managed by one or more medical treatment or clinical research protocols or regimens involving pharmaceutical drugs, physiological data, educational content, and health status assessment or quality of life questionnaires. This method of mass customization of medical protocols, present in a medical database, is useful in efficiently monitoring and managing medical outpatients, through simultaneously instructing selected patient subgroups present in the database to make a change in their respective medical protocols. These changes are to be applied to all members of the selected medical subgroup; and in addition the method provides for specific patient instructions for the individualized components of the medical protocols. This method may be repeatedly applied to the medical patient database system by selecting different subgroups defined by various different medical parameters. This method facilitates management or change of the many patients' protocols found in the database records, by allowing selected subgroups of medical outpatients to all receive the same change in a medical protocol, but requiring only one change process by the health care provider or database administrator. In addition, software-driven search engines can search other databases for subgroup-specific medical education content, pull this content into the database that is linked to the remote patient monitoring devices, and then the subgroup-specific content can be downloaded into the remote monitors to assist patients in their health management. In addition, end users can mass customize additional databases that they operate by importing into their respective databases the mass customized database disclosed in the present invention, thereby enhancing and expanding the applications served by their pre-existing database. By using the mass customization method whereby subgroup selected changes are combined with individualized changes, both population management and individual patient management goals may be realized.

Various electronic, devices may be used to monitor and manage medical treatment regimens and protocols for treating a patient's medical condition, These devices store one or more of the following: medication schedule data, treatment data, medical education content, patient query data and patient response data. The devices include a controller for controlling modes of device operation, controlling access to the memory, controlling display treatment data and the patient query data on a display, receiving and processing patient response data, tracking timing, and providing scheduled medication alarm signals. The devices may include two or more soft function keys interfaced with the controller. The soft function keys signal the controller, commanding it to execute different modes of operation of the medical monitoring device. The devices may also provide for scheduled medication alarm signals that alert the user concerning prescribed medications due to be taken.

While using various devices to monitor outpatients with chronic illnesses, and among clinical drug trial participants, patients often face complicated medical treatment protocols or regimens. These protocols require the patient to carry out a detailed series of events, in a sequential fashion throughout the day, related to taking medication, following other instructions e.g. taking their blood pressure or blood glucose levels and transmitting these values to a remote database), answering questions that assess their health status, and other events that need to be prompted and recorded. Some of these events and information are population-specific, and apply o entire populations of patients with specific diseases. Other events and information apply to sub-populations of patients, and still other events are specific to the individual patients themselves and present specific elements of the protocol that apply only to them and to no other patient.

What is missing from the previous methods, yet desirable for the medical personnel, caregivers, or family members monitoring and managing outpatients, is the ability to readily and easily mass customize tile information Provided to those patients, and the information to be captured from those patients. Such mass customization would enable patients and those who monitor them to insure that patients get the right information at the right time, to sequentially follow a complex medical protocol, and in-turn that the patients get the benefit of both population based and individualized monitoring and treatment.

It is therefore the object of the present invention to enable those medical personnel and caring family members to select and provide information to populations of patients, for example educational content and disease-specific questionnaires to a population of patients with congestive heart failure to subpopulations of patients, for example patients with congestive heart failure on a specific medication or combinations of medications' and in addition be able to provide each individual patient with information that is specific to them, for example medication using instructions, and individualized educational content and questionnaires.

It is the further object of this invention that the provision of this mass custormized information be enabled by a remotely accessible database that contains the patient files. Each patient file is constructed in such a fashion that it may be assigned population-specific data, fol ample educational content and questionnaires that relate to an entire population or subpopulation of patients, and simultaneously provide for the ability to enter into the patient file specific educational content for that specific patient (e.g. nutritional information), specific medication dosing instructions, specific queries for the patient regarding their health status and quality-of-life, and any other information of value to the management or monitoring of the patient or population of patients.

It is the further object of this invention that the patient file may receive information that is retrieved from other databases; via the use of search engines that search other databases for content that is specific to a population or sub-population of patients. This search and retrieve function can be applied to medical education content, self-management instructions, medication-specific dosing or side-effect information, self-diagnosis algorithms or questionnaires, drug interaction information and warnings, or any other information that will improve the medical outcome of the patient by delivering it to a specific population of patients.

It is the further object of this invention that the mass customized database, once created and placed into use, can be imported into other pre-existing databases that are used to monitor and manage patients, or used for statistical analysis in clinical trials, such that these other database users benefit from the addition of the mass customization feature and data to their particular database application.

It is the further object of this invention that the patient file be able to be remotely created, by accessing the database via direct dial-in or Internet access; and that the patient file, once created, be remotely transmitted to a series of possible patient monitors, including personal digital assistants (PDAs) such as the Palm Pilot, cellular telephones, pagers, interactive televisions, and custom-manufactured medical monitoring devices such as the Medi-Monitor system. The remote creation of the patient file could be accomplished by accessing the database from the patient monitor itself, a separate personal computer or PDA, a “thin-client” Internet terminal, or other means.

Each monitor may, but need not, have associated with it medication compartments that communicate with the monitor or clip onto it, such that the compartments have sensors that sense when the medication is being removed, and communicate this information to the memory of the device, or directly to the database.

Once the patient file is transmitted to these monitoring devices, the monitoring device firmware converts the file into a series of messages and queries to assist the patient in following the protocol, by prompting and monitoring a series of events throughout the day. In the alternative, a wireless signal can carry the application software and the patient file for direct insertion into the wireless device. The patient then interacts with the monitoring devices, which in turn communicate the collected patient information back to the database for report-generation to the medical personnel, caregivers and family members who are monitoring and managing the patients. New mass customization of the patient files can then take place based upon the collected it from the patient. In this fashion a complex protocol that requires a sequential set of actions id monitoring activities can be managed.

The sub-populations or groups of patients may be any group of patients that share one or more common characteristic that may effect or modify their medical condition or treatment protocols. Among the groups to consider are defined by age, gender, occupation, disease state, medical history event, medication category, specific medication, medication dosage, patient physiological measurement, for example weight, blood pressure, pulse rate, glucose level, any antigen level, pH, pO₂, temperature, EKG rhythm, pO₂ saturation of the blood, hormone level, or any psychological measurement, for example the score based upon standardized or nonstandardized tests measuring anxiety, stress, anger, suicidal tendencies, schizophrenic relapse, rapid cycling bipolar relapse or confusion. The groups may be age, gender, race, national origin, geographic location related in combination with a medical condition. The group may be based upon the same or similar disease state or medical condition, for example congestive heart failure, hypertension, angina, circulatory inadequacy or other disease condition of the cardiopulmonary and circulatory systems. The group may be based upon specific organ failure or dysfunction or upon a transplanted organ, for example asthma for the lungs, kidney failure for the kidney, arteriosclerosis or atherosclerosis of the heart, and transplantation of lung, kidney or heart. The group may be passed upon class of pathogen, or a specific pathogen. For example the group may )e based upon Viral infection, or upon a specific viral disease such as HIV/AIDS, hepatitis A, B, C, D, E or G. Similarly, the group may be based upon a bacterial disease agent, for example tuberculosis, malaria, cholera and meningitis. Groups may be defined by a specific type of microbial agent such as a virus, bacteria, mycotic infection and parasitic infection The group nay be based upon the type of pathology involved or the physiological system effected; for example cancer, autoimmune disease, muscular-skeletal system, or pathology of the hemopoetic, circulatory, reproductive, digestive, endocrine or nervous systems.

The groups may be based upon two or more characteristics as parameters for defining the group, for example HIV/AIDS and immune function, liver dysfunction and hepatitis, or bone degeneration and arthritis. The groups may be based upon any patient parameter available to the medical database, either directly in the database or accessible through linkage to another database or databases, and associated with a specific medical condition. Among the data in the database ire name of patient's physician, physician specialty, hospital, insurance company, type of insurance, diagnosis, length of illness, family history or previous medical history. The group may be based upon the type of clinical setting, for example hospital, emergency clinics, physician's office, institutional setting or home. The group may be based upon the name of the corporation, organization, physician, clinical research organization, pharmaceutical company, case worker or sponsor.

In each situation, the patients' medical protocols will be changed based upon the patients' inclusion in such a group. The change submitted to the group database files will modify such patient's medical protocol in the same manner. Each individual patient may be a member of any different sub-groups, each of which may have a change entered into the database. The hanged patient protocol is then uploaded to the monitoring device. Such uploading may be immediate, or it may be at the next scheduled uploading event. Notice of the uploading of a change is made through the monitoring device to alert the patient to the change in protocol.

In addition each individual patient's protocol may be modified in the database by entering into the individual patient file the elements of a medical protocol specific to that patient, for example medication type; medication dosage, dietary regimen, specific reminders, such as when to obtain a medication refill, when to call the doctor, and any algorithm-driven events that are based upon data inputted by the patient into the remote monitoring device, for example instructing the patient to dial “911” if they are having chest pain and have taken too much bronchodilator medication.

The database software may be provided by any operating system, for example those provided by Oracle and Microsoft.

The mass customization of the patient files, which are then translated into operating routines in remote monitors, can be accomplished utilizing a standard ODBC compliant database. The example shown below, written in Microsoft SQL Server 7, is illustrative, and should in no way be construed as limiting the invention. The following are screens used to collect data from patients, healthcare providers, clinicians and administrators regarding database parameters. These screens are present to illustrate one of the methods used in the present invention to populate the database with patient characteristics that may be used to create specific groups in later modifying the information in the databases.

A message will appear listing the user to accept appending a record to the ConfigFile table. Select “Yes,” Afterwards, the “Configuration File Processed” message will be displayed.

The ConfigID and the time of the configuration generation will be stored in the ConfigFiles table automatically when this procedure is run.

As illustrated in the above database entry fields, the goal of mass customization of patient protocols in clinical drug trials and in outpatient medical management is facilitated. The addition of pull-down menus to the fields, that populate the fields with standardized questionnaires and educational content further simplifies the mass customization method. Other fields are then individually populated with patient-specific content to complete the patient's medical Protocol design.

The database application of the mass customization system, in this case provided in a Microsoft SOL Server 7 database, can be converted into configuration files that are downloaded via modem, cable, or wireless means into the patient monitors. The files are translated into routines that convert the medical protocol structure into a series of prompt and record events. In this way the patients get the benefit of both population-based as well as individualized assessment and/or treatment protocols on a real-time basis.

Once the caregivers populate the data fields in the database, which converts these fields into configuration files that are downloaded into the monitors, the patient management protocols can be instantaneously updated. Through this method, the caregivers need not develop new software each time they want to change the database or patient protocols, and can rapidly take advantage of newly discovered population-based medical evidence to be communicated to patients, to give patients the advantages of up-to-the-minute medical knowledge without any delay caused by reprogramming or creating new software.

Alarm-based educational content messages, alarm-based treatment instructions, and population-based questionnaires can be assigned to specific groups of patients as previously disclosed. In addition, each individual patient file can be programmed for specific medication instructions by populating the appropriate fields.

In addition the patient file may receive information that is retrieved from other databases' via the use of search engines that search other databases for content that is specific to a population or sub-population of patients, or to an individual patient. The search engine can carry our its search based upon a single characteristic of a population, two or more simultaneous characteristics of a sub-population, or a combination of specific characteristics that are particular to an individual patient. This search and retrieve function can be applied to medical education content, self-management instructions, medication-specific dosing or side-effect information, self-diagnosis algorithms or questionnaires, drug-interaction information and warnings, or any other information that will improve the medical outcome of the patient by delivering it to a specific population of patients.

Thus a mass customization of patient management protocols is facilitated by the above database design; and implemented by downloading into monitors that translate the databased data into protocolized routines.

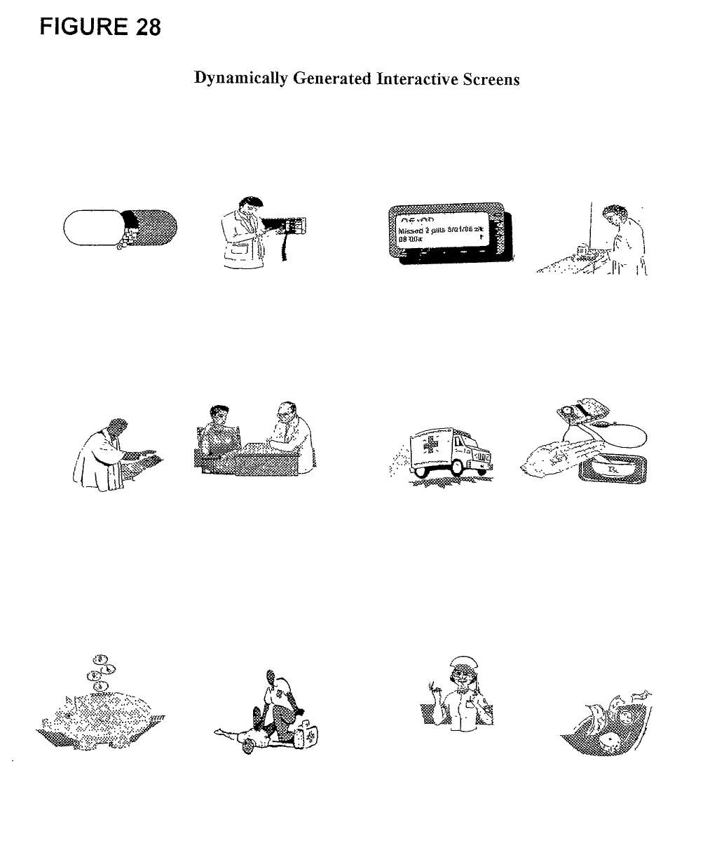

Any monitoring device capable of communicating by modem, fax, phone line or wireless means may be used to collect the patient data that is communicated to the database for storage and for display. Among the monitoring devices is the Medi-Monitor® described in PCT patent application WO98/18909 herein incorporated by reference. In the present invention, the Medi-Monitor® described may be further modified by placing the software and key functions and related software is a device not containing medication compartments. The medication compartments may be present in a separate associated device that contains compartments that indicate which compartment contains the medication and communicates by wireless means with the monitor containing the screen. Thus one type of Medi-Monitor® may have a separate component that communicates by wireless means with the monitor portion and may contain from 2 to 25 different medication compartments, more preferably 2-15 medication compartments, and most preferably 3-10 medication compartments as illustrated in FIG. 28.

The database containing the patient information and protocols is the site of the group changes made by the methods of the present invention. The database incorporating the group changes then uploads the modified protocol to each patients monitoring device for use by the patient as directed.

B. Time-and-Event Driven Medical Treatment Plan

In an embodiment of the present invention, system 100 provides real-time, time-and-event-driven medical treatment plans to outpatients and their caregivers preferably, but not necessarily, as described in commonly assigned U.S. patent Ser. No. 60/207,148, filed May 26, 2000, (herein referred to as the '148 application). The disclosure of the '148 application is incorporated herein by reference as though set forth in its entirety.

The present invention describes methods and apparatus useful in translating a complex medical treatment plan of a medical outpatient into a sequential series of automated, prompt and record events presented over time. In addition, individual patient medical treatment plans may be remotely created, modified, or viewed depending upon role-based assignments that permit different levels of access to and modification of the patient's time-and-event-driven medical treatment plan, depending upon the assigned role of the caregiver in the patient's treatment. The method has application in creating and linking medical databases containing data points that define the outpatient's medical treatment plan or protocol, with devices that can prompt the outpatient to carry out the sequential steps of a medical treatment plan, in proximity to the database, or while mobile at remote locations from the database. In addition the method provides for the treatment plan to be presented over finite time periods, with pre-defined time windows within which the patient must execute the treatment plan. In addition the method provides for recording whether the patient is following the treatment plan, while monitoring the progress of treatment. These outpatients may be managed by one or more medical treatment or clinical research protocols or plans that involve; pharmaceutical drugs, physiologic data, treatment instructions, medical educational content, medication compliance assessment, and health status or quality of life assessment. Each of these protocols or plans is first translated into a set of data points stored in a medical database and configured as a patient medical file. Role-based assignments determine who has access to the database and patient file their level of access, and their level of read and write, or read-only privileges, to provide different levels of database security and patient confidentiality. The data points are then translated into a sequence of time-and-event-driven, graphic and/or auditory, real-time communications presented to the patient or caregiver via the remote device that is linked to the database, The patient and/or caregiver then interact with the remote device that records these interactions in real-time, and communicates them to the database in real-time or via store-and-forward means. This method facilitates converting a complex medical treatment plan into a series of simple steps presented by the remote prompting and monitoring device, to assist patients and their caregivers in proper health management. By using the real-time, time-and-event-driven method, individual patient management goals and improved patient treatment outcomes may be realized by outpatients with chrome and complex conditions.

Various electronic devices may be used to monitor and manage medical treatment regimens and protocols for treating a patient's medical condition. These devices communicate one or more of the following: medication schedule and instruction data, medical treatment data, medical education content, patient query data and patient response data, and physiologic data. The devices include a controller for controlling modes of device operation, controlling access to the memory, controlling the communication of treatment data and patient query data on a display or via voice communications means, receiving and processing patient response data, tracking timing, and providing scheduled medication alarm signals. The devices may include soft function keys interfaced with the controller. The soft function keys signal the controller, commanding it to execute different modes of operation of the medical monitoring device. The devices may also provide from scheduled medication alarm signals that alert the user concerning prescribed medications to be taken.

While using various devices to monitor outpatients with chronic illnesses, and among clinical drug trial participants, patients often face complicated medical treatment plans or protocols. Further, caregivers may prescribe elements of a medical treatment plan or protocol by selecting a finite number of elements from an infinite number of available elements. Patients with more than one medical condition often consult multiple physicians, each physician then producing and prescribing a separate medical treatment protocol. These multiple protocols then frequently require active and mobile patients to sequentially carry out a highly detailed series of complex events throughout the day. These events may be related to taking medication, following other instructions (e.g. taking blood pressure or blood glucose levels and transmitting these values to a remote database), answering questions that assess their health status, and managing other treatment-related events that need to be prompted and recorded.

Despite the apparent miracles provided by an increasing array of modem medical treatments and medications, there is nonetheless a “dark side” created by the growing complexity of these regimens, which provides enormous challenges to patients and their caregivers who must precisely follow the complex medical plan to achieve good health outcomes. For example, patients with congestive heart failure, HIV/AIDS, or other chronic and complex conditions may have to manage well over 100 specific treatment-related-events in a given day. If improperly managed, the patient's illness may worsen, or death might ensue. Properly managed, the patient will have a good outcome and health will be maintained.

Given the infinite number and combinations of potential treatment elements available to include in a medical treatment plan, and the rowing complexity of these plans, software-driven databases are used to organize, manage, and communicate the treatment. While the patient's caregiver may try to select from an infinite number of data elements, a finite number of elements, to be combined and included into the protocol or plan, and thus program the database and receive reports on patient status and outcome, this is not easily accomplished. A fixed number and type of database screens and reports often may not accommodate this protocol complexity.

There is also increasing concern that medical treatment plans contained and managed in a database may violate patient confidentiality, through unauthorized disclosure of the medical treatment plan or results of treatment to individuals who have no right to that information. Another concern is that an unauthorized person may enter the database and alter a patient's medical treatment plan, delivering unauthorized instructions to the patient that could worsen illness of cause death.

What is missing from the previous methods, yet desirable for patients, medical personnel, caregivers, or family members monitoring and managing outpatients, is the ability to readily and easily convert a complex medical treatment plan into a series of simple prompt-and-record events that get communicated to the patient through time. Further, it is desirable that the database and its user interface be constructed in such a way that an infinite number of potential medical treatment data elements be easily combined as a finite number of elements used to populate database fields, and easily converted into a series of patient status reports. What is also desirable is that each of these participants (“Users”) is the patient's treatment be assigned role-based access to the database and patient file containing and driving the medical treatment plan, such that the patient controls the role-assignment and access either in person or through a designated caregiver to insure safety and confidentiality.

It is therefore an object of the present invention to translate a complex medical treatment plan of a medical outpatient into a sequential series of automated, real-time, time-and-event driven, prompted and recorded events presented over time to the patient and caregiver via a communication device that may be remotely located facilitating mobile information transfer, or adjacent to the database.

It is a further object of the present invention that the medical protocols may reside in the database, and be transferred into the memory of a remote device. The remote device contains software or firmware that translates the stored medical protocol into a sequential series of prompt and record communication events over time, that are then sequentially delivered to the patient via the device.

It is a further object of the present invention that the medical protocols reside in the database, and be transferred as a sequential series of automated, real-time, time-and-event-driven signals to a remote device. The device then converts the signals into real-time, prompt-and-record communication events that are delivered to the patient.

It is a further object of the present invention that the medical protocols may be delivered to the patient in the form of sequential, real-time, time-and-event-driven visual communications, audible communications (e.g. voice or music), or some combination thereof

It is a further object of the present invention that each of these protocols or plans is first translated into a set of data points stored in a medical database and configured as a patient medical file An individual patient medical treatment plan is created by selecting a finite combination of data elements from an infinite pool of data elements. Each data element is related to a pharmaceutical drug, physiologic parameter, treatment instruction, medical educational content, medication compliance, diagnostic test, laboratory test, drug-interaction, medical or surgical procedure, medical consultation, physical examination, health status, quality of life, patient queries, patient peer-group comparisons, patient outcome report, and any other component of a complete medical treatment plan or protocol that must be followed by the patient to ensure an optimum treatment outcome. The selected combination of data elements is readily and easily entered into the fields of a database, and converted to a set of prompt-and-record events, and reports.

It is a further object of this invention that the database stores a list of valid values (time range of acceptable values) for each of the data elements that comprise the medical treatment plan, and for each of the prompt-and-record events that signify the patient's response to and adherence to the treatment plan. The valid values may be selected from a menu of possible choices, or determined for each individual patient or population of patients by a physician, pharmacist, or other caregiver. The time period of valid values may be from 10 seconds to 24 hours more narrowly it may be from 10 seconds to 12 hours, more narrowly it may be from one minute to 8 hours, and yet more narrowly it may be from 2 minutes to six hours; and yet more narrowly it may be from 3) minutes to 6 hours; and still more narrowly it may be from 4 minutes to 4 hours, and even more narrowly it may be from 5 minutes to two hours, and still more narrowly it may be from 5 minutes to one hour. When any of the responses or data elements stored in the database, (whether medical treatment protocol data or patient event data), fall outside the range of valid values for that particular data element, further communication is provided to the user, caregiver, or patient. This further communication indicates that the specific data element has deviated from the valid value or range of acceptable values. While the events in the protocol may start sequentially, there may be two or more events occupying the same time frame or valid value. There also may be partial overlap of valid value time periods for separate events.

It is a further object of this invention that among the patient medical treatment events are vital signs and other biological measurements that are made using separate physiological monitoring devices whose use is prompted by the communicating monitoring device, whereupon the physiological value is either entered manually into the communicating monitoring device for communication to the database, or alternatively, the physiological monitoring device communicates separately with the database. Among the common physiological monitoring devices are weight scales, blood pressure, glucose, temperature, respirometers and pulse rate devices.

It is a further object of this invention that these individual patient medical treatment plans may be remotely created, modified, or viewed depending upon role-based assignments that permit different levels of access to, and modification of, the patient's time-and-event-driven medical treatment plan, depending upon the assigned role of the caregiver in the patient's treatment, and the specific user of the information system. Role-based assignments determine which user has access to the database and patient file, their level of access and their level of read and write, or read-only privileges, to provide different levels of database security and patient confidentiality. Dynamic content generating software can determine which user receives specific Screens for access and data entry.

It is a further object of this invention that role-based assignments in the database determine which service or combination of services are made available to the user, patient or caregiver, including, but not limited to, compliance monitoring, physiologic monitoring personal emergency response monitoring, patient counseling, patient performance reports that indicate how well the patient is following the medical treatment plan, patient peer-group-based performance/outcomes reports, drug-interaction reports (prescription, over-the-counter, herbal, nutriceutical, vitamin, etc.), and other reports. Using a menu-driven or other form of registration, the database business logic and rules will determine for each user the role-based assignment of access to services. Dynamic content generating software will generate the specific reports for the specific user, based upon their assigned role. The reports could be accessed from the database via an Internet, Extranet, Intranet, direct dial-in, wireless broadcast, taxed, mailed, or telephoned means of communication. In this way each user's role-based assignment can be commercially priced, based upon the service or combination of services assigned to the specific role.

The data elements are translated into a sequence of time-and-event-driven, graphic and/or auditory, real-time communications presented to the patient or caregiver via the remote device that is linked to the database. The presentation may be linear or non-linear, in that some of the events may overlap in time. The patient and/or caregiver then interact with the remote device that records these interactions in real-time, and communicates them to the database in real-time or via store-and-forward means.

It is a further object of this invention that the patient file be able to be remotely created, by accessing the database via direct dial-in or Internet access; and that the patient file, once created, be remotely transmitted to a series of possible patient monitors, including personal digital assistants (PDAs) such as the Palm Pilot, cellular telephones, pagers, interactive televisions, and custom-manufactured medical monitoring devices such as the Medi-Monitor® System. The remote creation of the patient file could be accomplished by accessing the database from the patient monitor itself, a separate personal computer or PDA, a “thin-client” Internet terminal, or other means.

Each monitor may, but need not, have associated with it medication compartments that communicate with the monitor or clip onto it, such that the compartments have sensors that sense when the medication is being removed, and communicate this information to the memory of the device, or directly to the database.

Once the patient file is transmitted to these monitoring devices, the monitoring device firmware, or other resident software, converts the file into a series of messages and queries to assist the patient in following the protocol, by prompting and monitoring a series of events containing the medical treatment plan into the prompting and monitoring events. Another alternative would be for a wireless signal to carry the application software and the patient file for direct insertion into the wireless device. The patient then interacts with the monitoring device, which in turn communicates the collected patient information back to the database for report generation to the medical personnel, caregivers and family members who are monitoring and managing the patients. The patient file may contain a range of events 2-7000 events, more preferably 3-1,000 events, even More preferably 4-700 events, and most preferably 4-300 events. In this fashion a complex protocol that requires a sequential set of actions and monitoring activities can be managed.

The data elements that comprise the medical treatment plan entered into the database may relate to instructions or medical educational content about specific medication, medication dosage, patient physiological measurement, for example weight, blood pressure, pulse rate, glucose level, any antigen level, pH, pO₂, temperature, EKG rhythm, pO₂ saturation of the blood, hormone level, any psychological measurement, for example the score based upon standardized or non-standardized tests measuring anxiety, stress, anger, suicidal tendencies, schizophrenic relapse, rapid cycling bipolar relapse or confusion; medical education content related to any disease state or medical condition, for example congestive heart failure, hypertension, angina, circulatory inadequacy or other disease condition of the cardiopulmonary and circulatory systems, specific organ failure, dysfunction of an organ or system or transplanted organ such as asthma for the lump, kidney failure for the kidney, arteriosclerosis or atherosclerosis of the heart, and transplantation of lung, kidney of heart; class of pathogen, or a specific pathogen, for example instructions or content may be based upon viral infection, or upon a specific viral disease such as HIV AIDS, hepatitis A, B, C, D, E or G. Similarly, the instructions or content comprising the data elements may be based upon a bacterial disease agent, for example tuberculosis, malaria, cholera and meningitis a specific microbial agent such as a virus, bacteria, mycotic infection and parasitic infection or may be based upon the type of disease or pathology involved or the physiological system effected for example cancer, autommiume disease, muscular-skeletal system, or pathology of the hematopoetic, circulatory, reproductive, dermatologic, digestive, endocrine or nervous system.

In each situation, the patients' medical protocols will be changed based upon the patients' condition, by entering into the individual patient file the elements of a medical protocol specific to that patient; for example medication type; medication dosage, dietary regimen, specific reminders, such as when to obtain a medication refill, when to call the doctor, and any algorithm driven events that are based upon data inputted by the patient into the remote monitoring device, for example instructing the patient to dial “911” if they are having chest pain and have taken too much bronchodilator medication.

The database software may be any functional database system, for example Oracle and Microsoft. The remote device may be any communications device such as a Personal Digital Assistant such as Palm Pilot®, cellular telephone, interactive pager, interactive television, or proprietary device such as the MediMonitor.™.

The creation of a complex medical treatment plan by selecting a combination of data elements from an infinite number of possible data elements, and the conversion of the medical treatment plan into a series of real-time, time-and-event-driven communications delivered over time and space to remote monitors, can be accomplished utilizing a standard Open Database Connectivity Standard (ODBQ object-oriented database compliant database. The example shown below, written in Microsoft SQL Server 7, is illustrative, and should in no way be construed as limiting the invention.

The database application, in this case provided in a Microsoft SQL Server 7 database, can be converted into configuration files that are downloaded via modem, cable, infra-red, laser, computer disk, or wireless means into the patient monitors. The files are translated into routines that convert the medical protocol structure into a series of prompt and record events in this way the patients get the benefit of both population-based as well as individualized assessment and/or treatment protocols on a real-time basis.

Once the caregivers populate the data fields in the database by selecting a combination of data elements for the medical treatment plan from an infinite number of possible data elements, they enter the data elements into fields of the database, The database converts these fields into configuration files that are downloaded into the patient devices or monitors and stored in their memories. In the alternative, the database may sequentially transmit the real-time prompt-and-record events directly into the patient devices. This method results in patient management protocols that can be instantaneously updated. Through this method, the caregivers need not develop new software each time they want to change the database or patient protocols.

Alarm-based medication events, educational content messages, alarm-based treatment instructions, questionnaires, and any other elements contained in the medical treatment plan or protocol can be assigned to specific groups of patients or individual patient-, to be delivered over time and space to the remote monitoring devices.

The events that are then prompted and recorded can be organized and managed in an event log as illustrated below, which corresponds with the appropriate fields of the database that is prepared to accept the events from the event log.

Any monitoring device capable of communicating by modem, fax, phone line or wireless means may be used to collect the patient data that is communicated to the database for storage and for display. Among the monitoring devices is the Medi-Monitorg described in PCT patent application WO98/38909 herein incorporated by reference. The database containing the patient information and protocols is the site of the medical treatment plans made by the methods of the present invention. The database incorporating the treatment plan then uploads the modified plan to each patient's monitoring device for use by the patient as directed by the real-time, time-and-event-driven method.

For example, using the above method, a complex treatment regimen for a heart failure patient may be created, converted into a series of prompt-and-record events, and be securely managed to preserve the confidentiality of the patient information. Heart failure patients may typically require the following data elements in their medical treatment plan, with information about each data element to be communicated at a particular time: a medication regimen comprised of a beta-blocker, ACE inhibitor, diuretic, and Di8oxin; dosing instructions for each medication, a description of each medication and rationale for taking the medication; what the medication looks like along with its color; daily measures of weight, blood pressure, pulse, and blood oxygen levels, a salt restricted, low fat, low calorie diet or an exercise program tailored to their specific level of heart failure, questionnaires that assess their shortness of breath, chest pain, energy level, and swelling of the legs; information updates about new drugs used to treat the condition, information about when to call the doctor with specific complaints or side effects or adverse drug reactions, and other information.

Using the above method, this highly complicated treatment plan can be converted into fields in the database, then into a series of real-time messages and recorded events that are communicated to the patient via the remote device, whereby the patient interacts with the device to respond to the messages and answer the queries' and whereby the remote device then communicates the patient responses to the database.

C. Risk-Stratified Triage and Medical Interventions

In an embodiment of the present invention, system 100 provides real-time, time-and-event-driven medical treatment plans to outpatients and their caregivers preferably, but not necessarily, as described in commonly assigned U.S. patent Ser. No. 60/214,688, filed Jun. 27, 2000, (herein referred to as the '688 application). The disclosure of the '688 application is incorporated herein by reference as though set forth in its entirety.

The present invention describes methods and apparatus useful in translating real-time recorded information from medical outpatients into analyzed and formatted information, and recommended treatment interventions, that are broadcast in real-time to the caregivers or family members of these outpatients. The recorded information may include medication compliance, health status, quality-of-life, physiologic, laboratory, or other medical data that is captured in real-time from medical outpatients, and stored in a database that hosts individualized and population-based complex medical treatment plans and/or medical protocols. The database provides for the creation of patient treatment plans by a mass-customization method, whereby individual patients can be assigned treatment unique to their clinical situation, and treatment based upon a group or population to which they belong. The database provides for: analysis of the real-time outpatient data captured from the patients; bio-statistical analysis and stratification of patients based upon their respective risks of developing worsening clinical outcomes, and increased medical costs; pattern recognition analysis of medical data to determine new correlation of data elements; and broadcast of real-time information to the respective caregivers and family members of the patients. The real-time information broadcast to the caregivers can include raw data and/or analyzed and formatted data recorded from the patients; patient deviations from acceptable population-based and/or individual patient standards; predictions of the clinical and economic risks associated with the respective patients' conditions; new patterns of correlation among medical and/or non-medical data elements; and guidelines for medical interventions. Medical decision support software may be used to analyze and correlate the data captured from the patient, and generate recommendations for treatment interventions. In addition, the content of the patient information broadcast to caregivers and family members may be based upon role-based assignments, depending upon the assigned role of the caregiver or family member in the patient's treatment. The broadcast information can be transmitted to portable wireless devices carried by the caregivers and family members, to their personal computers, to their interactive televisions, or to other information appliances. The method has application in linking real-time medical information captured from patients with medical databases, which in turn are linked to caregivers and family members via the Internet. The database analyzes that information, stratifies patient risk, determines new correlation between data elements provided to and captured from the patient, and determines appropriate medical interventions; then provides “push technology” to broadcast relevant information at the correct time, to prompt the caregiver or family member to carry out or modify sequential medical interventions with the patient. After a review of the analyzed data, the caregiver or family member can then update the patient's medical treatment plan with new instructions, or communicate new instructions directly to the patient. When patients and their family members and caregivers carry wireless devices that are linked with the database via the Internet, the entire information system operates in real-time. By using this real-time analysis and triage method, individual patient management goals, improved patient treatment outcomes, real-time outcomes research, and reduced medical costs may be realized by outpatients with chronic and complex conditions.

Various electronic devices may be used to monitor and manage medical treatment regimens and protocols for treating a patient's medical condition. These devices communicate one or more of the following to the patient: medication schedule and instruction data, medical treatment data, medical education content, patient query data and patient response data, and physiologic data. The devices may record information from the patient related to medication compliance behavior, health status, quality-of-life, physiologic, laboratory, or other medical data. The devices include a controller for controlling modes of device operation, controlling access to the memory, controlling the communication of treatment data and patient query data on a display or via voice communications means, receiving and processing patient response data, tracking timing, and providing scheduled medication alarm signals. The devices may include soft function keys interfaced with the controller. The soft function keys signal the controller, commanding it to execute different modes of operation of the medical monitoring device. The devices may also provide for scheduled medication alarm signals that alert the user concerning prescribed medications to be taken.

Various medical devices also, via the Internet, link caregivers with clinical, practice management, and research databases, such that the caregivers can access the electronic medical record of a patient, access laboratory and radiological test results, prescribe for the patient, and otherwise conduct medical care from a site distant to the patient.

Despite the miracles provided by an increasing array of modem medical treatments and medical information technologies, the growing complexity of medical treatment regimens provides enormous challenges to patients and their caregivers who must precisely follow the complex medical plan, and closely monitor the patients' health status, to achieve good health outcomes. At the same time the caregivers frequently suffer from an overload caused by receiving too much information. For example, patients with congestive heart failure, HIV/AIDS, or other chronic and complex conditions may have to manage well over 100 specific treatment-related-events in a given day. If improperly managed, or not closely monitored, the patient's illness may worsen, or death might ensue. Properly managed, the patient will increase the prospect of a good outcome, and health will be maintained. At the same time it is a daunting challenge for family members and caregivers of these patients to effectively and remotely monitor and manage the patient's condition, and make changes to the patient's medical treatment regimen. Many patients with these chronic conditions are medically stable for long periods of time, yet may show signs of deterioration well in advance of life-threatening events. To report all of the patient-based monitoring data to caregivers or family members would quickly overwhelming them.

There is also a need to more rapidly assess the impact of complex treatment regimens, including positive effects such as improved health, and negative effects such as adverse drug reactions, through improved methods of outcomes research.

There is also increasing concern that patient data contained and managed in a database may violate patient confidentiality, through unauthorized disclosure of the medical treatment plan or results of treatment to individuals who have no right to that information.

Given the infinite number and combinations of potential treatment elements available to include in a medical treatment plan, and the growing complexity of these plans, software-driven databases are used to organize, manage, and communicate the patient's treatment, and store the patient's responses to treatment. The databases may provide for the creation of patient treatment plans by a mass-customization method, whereby individual patients can be assigned treatment unique to their clinical situation, and treatment based upon a group or population to which they belong.

How do the caregiver or family member easily receive real-time broadcasts of only those reports on patient status and health outcome that are significant? How does the caregiver compare these reports of the patient's condition to normal or acceptable values for that patient or population of comparable patients? How does the caregiver determine the probability of clinical deterioration in the patient, and the likely increase in medical costs thereby? How does the caregiver then decide the specific timing and nature of the medical intervention that has the best likelihood of improving the patient's condition? How is the patient assured that the broadcast data is kept confidential, and is sent only to those who “need to know?” How does the caregiver recognize new patterns of correlation among medical treatment interventions and patient outcomes?

What is missing from the previous methods, yet desirable for patients, medical personnel, caregivers, or family members monitoring and managing outpatients, is the ability to easily and automatically alert them to unacceptable changes in the patient's condition. It is desirable to convert complex medical data that is captured from the patient into a sequence of broadcasts of simple patient status reports, that get communicated to the correct caregiver or family member at the correct time. What is also desirable is that the patients are “triaged,” such that the caregiver will only be broadcast relevant information, based upon when the data based-analysis of the patient data predicts a poor outcome for the patient, and predicts increased costs to the healthcare system. What is also desirable is that each of these participants (“users”) in the patient's treatment be assigned role-based access to the broadcast information, such that the patient controls the role-assignment and access either personally or through a designated caregiver, to insure safety and confidentiality. What is also desirable is that the effects of treatment interventions on the patient be rapidly analyzed to pick up new patterns of correlation between medical and non-medical data elements related to the patient's condition and medical treatment plan.

It is therefore an object of the present invention to translate complex, real-time medical data recorded from a medical outpatient and stored in a database into a sequential series of automated, real-time messages, broadcast over time to a communication device possessed by the patient's designated family member and/or caregiver. The messages may be presented in textual, graphical, pictorial, or voice formats. The messages may include raw patient data, or reports that are easy and quickly understood. The communication device may be remotely located from the patient and/or database, facilitating mobile information transfer, or be located adjacent to the patient and/or database.

It is a further object of the present invention that the medical data recorded from the patients be subjected to bio-statistical and outcomes analysis, such that the real-time messages broadcast to the caregiver include the benefit of this analysis. The analysis may include a risk-stratification of the patients being monitored, probability of patient decline, likely increase in costs associated with that decline, and other information that serves to distinguish stable from unstable patients, based upon population-based outcomes research. The present invention thereby provides a medical triage function permitting rapid intervention for those patients identified as most in need of attention.

It is a further object of this invention that the database stores a list of valid values (range of acceptable values) for each of the data elements that comprise recorded events that signify the patient's response to and adherence to the treatment plan. For example each user could preset an acceptable range of minimum and maximum values for a patient's medication compliance behavior, answers to health status questions, weight, blood pressure, and other values. The valid values may be selected from a menu of possible choices, or determined for each individual patient or population of patients by a physician, pharmacist, or other caregiver. The valid values may also be based upon population-based or individual patient standards. When any of the data elements stored in the database, (whether medical treatment protocol data or patient event data), fall outside the range of valid values for that particular data element, further communication is broadcast to the user, caregiver, or patient. This further communication indicates that the specific data element representing the patient's behavior or condition has deviated from the valid value or range of acceptable values.

It is a further object of the present invention that the medical decision support protocols may reside in the database, and be activated once the inputted patient monitoring data correlates with a corresponding protocol. The decision support protocol would then be transferred to the medical caregiver as a sequential series of automated, recommended interventions via signals to a remote device or a device proximate to the database and/or patient. The device then converts the signals into real-time messages that are delivered to the caregiver.

It is a further object of the present invention that the broadcast messages may be delivered to the family member and/or caregiver in the form of sequential, real-time visual communications, audible communications (e.g. voice or music), or some combination thereof.

It is a further object of this invention that these broadcast messages about an individual patient or population of patients may be viewed depending upon role-based assignments that permit different levels of access to the messages broadcast from the database, depending upon the assigned role of the caregiver in the patient's treatment, and the specific user of the information system. Dynamic content generating software will generate the specific reports for the specific user, based upon their assigned role.

It is the further object of this invention that real-time outcomes research be conducted on patients, via pattern recognition software that examines correlation between medication compliance, health status, quality-of-life, physiologic, laboratory, treatment intervention, or other medical data that is captured in real-time from medical outpatients, along with non-medical data such as demographic characteristics, genotypic analysis, history of treatment, or any other pertinent data relating to the patient.

It is a further object of this invention that once the family member or caregiver receives the real-time broadcast messages in an information device (which may include a personal computer, personal digital assistant [PDA] such as the Palm Pilot®, cellular telephone, pager, or interactive television). They may then use the information device to remotely modify the patient file in real-time, via the connectivity provided through Internet access to the database. The patient file, once modified, may be remotely transmitted to a series of possible patient monitors, including personal digital assistants (PDAs) such as the Palm Pilot®, cellular telephones, pagers, interactive televisions, and custom-manufactured medical monitoring devices such as the Medi-Monitor® System. In the alternative, the caregiver or family member, based upon the information they receive about the patient, may directly communicate with the patient in real-time, to provide live instructions, interventions, and/or queries.

By way of example, a female patient with congestive heart failure (CHF) may record in a portable wireless information device (WID) that they have missed two doses of medication that improves the efficiency of heart function, and one dose of medication that reduces blood pressure. In addition they have had two episodes of moderate chest pain in the past eight hours; as well, they indicate that their weight has increased by three pounds in the past twenty-four hours. This data is communicated to the database, where it is analyzed, and determined that the patient has a 75% chance of requiring hospitalization in the next 72 hours, at an average cost of $25,000 for the hospitalization. The database further analyzes the data and determines that the best treatment intervention for the patient is to give them intravenous diuretics, and two nitroglycerin tablets, in the doctor's office. The database also determines a new pattern recognition or correlation, whereby patients that miss two or more doses of the medication used to improve the efficiency of heart function are three times as likely to gain weight compared to patients who only miss one dose of the medication. The database broadcasts the risk-analysis, cost analysis, new pattern recognition, and treatment intervention information to the doctor's wireless device. Simultaneously, the CHF patient's daughter is broadcast a simple message, via her cellular phone, that her mother has missed medication and is in need of medical attention.

The database software may be any functional database system, for example Oracle and Microsoft. The Business Logic Rules portion of the database may be created by applications written for a variety of commercially available software products. The risk analysis software, for example, could be an application written for the MEDSTAT Corporation Disease Staging Software. The decision support software for medical interventions could be an application written for the Apache Medical Systems Voyager+Software. The software that enables new pattern recognition among data elements; and enables the caregiver or family member to create and modify valid value ranges for patient-based monitoring data, that determine and control the timing and content of the broadcast messages, could be an application written for Synera Corporation Discovery Software. The broadcast software could be an application written for the Mobile Application Platform developed by Phone2 Networks, Inc. The remote devices may be any communications devices such as a Personal Digital Assistant such as Palm Pilot®, cellular telephone, interactive pager, interactive television, or proprietary device such as the Med-eMonitor™.

The creation of a mass-customized patient database that converts a medical treatment plan into a series of real-time, time-and-event-driven communications delivered over time and space to remote monitors; and provides for data analysis and reports to caregivers and family members; can be accomplished utilizing a standard ODBC (Object-oriented database compliant database. The example shown below, written in Microsoft SQL Server 7, is illustrative, and should in no way be construed as limiting the invention.

The database application, in this case provided in a Microsoft SQL Server 7 database, can be converted into configuration files that are downloaded via modem, cable, infra-red, laser, computer disk, or wireless means into the patient monitors. The files are translated into routines that convert the medical protocol structure into a series of prompt and record events. In this way the patients get the benefit of both population-based as well as individualized assessment and/or treatment protocols on a real-time basis.

Once the caregivers populate the data fields in the database by selecting a combination of data elements for the medical treatment plan from an infinite number of possible data elements, they enter the data elements into fields of the database. The database converts these fields into configuration files that are downloaded into the patient devices or monitors and stored in their memories. In the alternative, the database may sequentially transmit the real-time prompt-and-record events directly into the patient devices. This method results in patient management protocols that can be instantaneously updated. Through this method, the caregivers need not develop new software each time they want to change the database or patient protocols

Alarm-based medication events, educational content messages, alarm-based treatment instructions, questionnaires, and any other elements contained in the medical treatment plan or protocol can be assigned to specific groups of patients or individual patients to be delivered over time and space to the remote monitoring devices.

The events that are then prompted and recorded can be organized and managed in an event log as illustrated below, which corresponds with the appropriate fields of the database that is prepared to accept the events from the event log.

Any monitoring device capable of communicating by modem, fax, phone line or wireless means may be used to collect the patient data that is communicated to the database for storage and for display. Among the monitoring devices is the Medi-Monitor® described in PCT patent application WO98/38909 herein incorporated by reference. The database containing the patient information and protocols is the site of the medical treatment plans made by the methods of the present invention. The database incorporating the treatment plan then uploads the modified plan to each patient's monitoring device for use by the patient as directed by the real-time, time-and-event-driven method.

For example, using the above method, a complex treatment regimen for a heart failure patient may be created, converted into a series of prompt-and-record events, and be securely managed to preserve the confidentiality of the patient information. Heart failure patients may typically require the following data elements in their medical treatment plan, with information about each data element to be communicated at a particular time: a medication regimen comprised of a beta-blocker, ACE inhibitor, diuretic, and Digoxin; dosing instructions for each medication; a description of each medication and rationale for taking the medication; what the medication looks like along with its color; daily measures of weight, blood pressure, pulse, and blood oxygen levels; a salt restricted, low fat, low calorie diet; an exercise program tailored to their specific level of heart failure; questionnaires that assess their shortness of breath, chest pain, energy level, and swelling of the legs; information updates about new drugs used to treat the condition; information about when to call the doctor with specific complaints or side effects or adverse drug reactions; and other information.

Using the above method, this highly complicated treatment plan can be converted into fields in the database, then into a series of real-time messages and recorded events that are communicated to the patient via the remote device, whereby the patient interacts with the device to respond to the messages and answer the queries; and whereby the remote device then communicates the patient responses to the database. Then the Event Details, Daily Dose Compliance, and other recorded patient-monitoring data can be broadcast to wireless information devices used by caregivers and family members to monitor the patients, receive risk-stratified data analysis and recommended interventions, and update the patient files with new instructions.

D. Self-Selected Synchronized Database-Linked Medical Monitors

In an embodiment of the present invention, system 100 provides self-selected, synchronized, database-linked medical monitors to outpatients and their caregivers preferably, but not necessarily, as described in commonly assigned U.S. patent Ser. No. 60/226,515, filed Aug. 21, 2000, (herein referred to as the '515 application). The disclosure of the '515 application is incorporated herein by reference as though set forth in its entirety.

The present invention describes methods and apparatus useful in translating a complex medical treatment plan of a medical outpatient into a sequential series of automated, prompt and record events presented over time; to two or more medical monitors used by a single patient. The medical monitors are linked to a database that updates and synchronizes the information presented to all of the monitors, and captures information from all of the monitors. Individual patient medical treatment plans may be remotely created, modified, or viewed in the database; and in turn are transformed by business logic rules and a communications system into prompted and recorded events that are delivered to the patient by two or more remote monitors. In the alternative, the patient selects a specific monitor that he or she will use at a specific time, and the database senses the monitor in active use by the patient and communicates with it directly. The method has application in creating and linking medical databases containing data points that define the outpatient's medical treatment plan or protocol, with multiple different devices with different characteristics, that can prompt the outpatient to carry out the sequential steps of a medical treatment plan, and record the patient's responses. The patient can self-select the monitor that he or she will be using for a particular lifestyle preference at a particular time. For example, the use of a larger bedside monitor that dispenses medication, while the patient is at home; a portable, pocket-sized personal digital assistant monitor for use in the office or while out at a meeting; a smart-phone monitor based in a cellular telephone, for use while driving; and one or more physiologic monitors to record the physical responses to medical treatment. With each different monitor used by the patient, continuous updates of the treatment plan contained in the database enable each of the monitors to receive the correct prompted events at the correct times. Dynamically generated screen configuration software enables the information presented over the monitors to be formatted in such a way that it is properly displayed and easily understandable, regardless of which monitor is in use by the patient. In addition the method provides for each device to record whether the patient is following the treatment plan; and to upload that recorded data to the database; which provides for monitoring the progress of treatment as the individual patient uses multiple monitoring devices. The monitored outpatient may be managed by one or more medical treatment or clinical research protocols or plans that involve; pharmaceutical drugs, physiologic data, treatment instructions, medical educational content, medication compliance assessment, and health status or quality of life assessment. Each of these protocols or plans is first translated into a set of data points stored in a medical database and configured as a patient medical file. The data points are then translated into a sequence of time-and-event-driven, graphic and/or auditory, real-time communications presented to the patient or caregiver via two or more different remote devices that are linked to the database. Regardless of which device is used, in the event of graphic communications, the formatting remains consistently presented to each one of the multiple device displays. The patient then interacts with two or more remote devices that record these interactions in real-time, and communicate the recorded data to the database in real-time or via store-and-forward means. This method facilitates converting a complex medical treatment plan into a series of simple steps presented by the remote prompting and recording facilitated by the monitoring devices, to assist patients and their caregivers in proper health management. By using the method of multiple medical monitoring devices synchronized by a central database, the patient will be able to properly carry out a complex medical treatment plan or clinical drug trial protocol involving the use of two or more monitoring devices. The patient will have greater freedom of choice regarding which device is used at a particular time, enabling better integration of the entire monitoring system into the patient's lifestyle, in turn resulting in better compliance by the patient with the medical treatment plan.

Various electronic devices may be used to monitor and manage medical treatment regimens and protocols for treating a patient's medical condition. These devices communicate one or more of the following: medication schedule and instruction data, medical treatment data, medical education content, patient query data and patient response data, and physiologic data. The devices include a controller for controlling modes of device operation, controlling access to the memory, controlling the communication of treatment data and patient query data on a display or via voice communications means, receiving and processing patient response data, tracking timing, and providing scheduled medication alarm signals. The devices may include soft function keys interfaced with the controller. The soft function keys signal the controller, commanding it to execute different modes of operation of the medical monitoring device. The devices may also provide for scheduled medication alarm signals that alert the user concerning prescribed medications to be taken.

While using various devices to monitor outpatients with chronic illnesses, and among clinical drug trial participants, patients often face complicated medical treatment plans or protocols. Further, caregivers may prescribe elements of a medical treatment plan or protocol by selecting a finite number of elements from an infinite number of available elements. Patients with more than one medical condition consult multiple physicians, each physician then producing and prescribing a separate medical treatment protocol. These multiple protocols then frequently require active and mobile patients to sequentially carry out a highly detailed series of complex events throughout the day. These events may be related to taking medication, following other instructions (e.g. taking blood pressure or blood glucose levels and transmitting these values to a remote database), answering questions that assess their health status, and managing other treatment-related events that need to be prompted and recorded.

Despite the miracles provided by an increasing array of modern medical treatments and medications, there is nonetheless a “dark side” created by the growing complexity of these regimens, which provides enormous challenges to patients and their caregivers who must precisely follow the complex medical plan to achieve good health outcomes. For example, patients with congestive heart failure, HIV/AIDS, or other chronic and complex conditions may have to manage well over 100 specific treatment-related-events in a given day. If improperly managed, the patient's illness may worsen, or death might ensue. Properly managed, the patient will have a good outcome and health will be maintained.

Given the infinite number and combinations of potential treatment elements available to include in a medical treatment plan, and the growing complexity of these plans, software-driven databases are used to organize, manage, and communicate the treatment. These databases frequently communicate with a single remote device used by the patient. However, each remote device has particular differences in functionality, strengths and weaknesses in functionality, and is better suited for particular aspects of the treatment plan and patient's lifestyle, while not being well suited for other aspects of the patient's treatment plan and lifestyle. Also, while it is not uncommon for a single device to cease functioning, or to function poorly, it is much less common for multiple devices to simultaneously breakdown.

What is missing from the previous methods, yet desirable for patients, medical personnel, caregivers, or family members monitoring and managing outpatients, is the ability to readily and easily convert a complex medical treatment plan into a series of simple prompt-and-record events that get communicated to and from the patient through time, over multiple different devices, in a synchronized fashion. Further, it is desirable that the database and its communicating devices be constructed in such a way that the patient can select which device he or she will use at a particular time, based upon device functionality, the patient's location, activity level, need for portability of the device, and other lifestyle requirements. Further, it is desirable that the database be the central repository for the patient's treatment plan, and for the patient's responses to treatment as recorded by the medical devices, such that the entire treatment plan can be delivered and monitored independent of a single device, but rather through multiple devices. Finally, it is desirable that the database be able to communicate the treatment plan or protocol to the patient over multiple devices with different functionality to accommodate complex informational and monitoring needs; and should a particular device cease to function; thereby ensuring greater patient safety.

It is therefore an object of the present invention to translate a complex medical treatment plan of a medical outpatient into a sequential series of automated, real-time, time-and-event-driven, prompt and record events presented over time to the patient via multiple monitoring and communication devices that may be remotely located, facilitating mobile information transfer to the patient.

It is a further object of the present invention that the medical protocols may reside in the database, and be transferred into the memories of the remote devices. The remote devices contain software or firmware that translates the stored medical protocol into a sequential series of prompt and record communication events over time, that are then delivered to the and captured from the patient via the devices.

It is a further object of the present invention that the medical protocols may reside in the database, and be directly transferred as a sequential series of automated, real-time, time-and-event-driven signals to the multiple remote devices. The devices then convert the signals into real-time, prompt-and-record communication events that are delivered to and captured from the patient.

It is a further object of the present invention that the medical protocols may be delivered to the patient in the form of sequential, real-time, time-and-event-driven visual communications, audible communications (e.g. voice or music), or some combination thereof, in a synchronized fashion, such that the patient will reliably be able to use a suite of devices, and needn't depend upon a single device to get the information; but may select one of multiple devices that will prompt, record and communicate with the database, thereby continually updating the patient data in the database.

It is a further object of the present invention that each of these protocols or plans is first translated into a set of data points stored in a medical database and configured as a patient medical file. An individual patient medical treatment plan is created by selecting a finite combination of data elements from an infinite pool of data elements. Each data element is related to a pharmaceutical drug, physiologic parameter, treatment instruction, medical educational content, medication compliance, diagnostic test, laboratory test, drug-interaction, medical or surgical procedure, medical consultation, physical examination, health status, quality of life, patient queries, patient peer-group comparisons, patient outcome report, and any other component of a complete medical treatment plan or protocol that must be followed by the patient to ensure an optimum treatment outcome. The selected combination of data elements is readily and easily entered into the fields of a database, and converted to a set of prompt-and-record events, and reports. These events and reports may then be communicated to patient monitoring devices such as a “smart phone” cellular telephone, a Personal Digital Assistant (PDA) such as the Palm Pilot®, or a proprietary device such as the Med-eMonitor Medication Dispenser; and/or various types of physiologic monitors that measure the patient's physical responses to treatment.

It is a further object of this invention that the patient can select one or more of the monitoring devices at a particular time based upon lifestyle considerations, and the database will communicate the correct information to the particular device at the correct time, based upon the patient's medical treatment plan. In this way the patient can maintain the privacy of their medical treatment plan. For example, when out in public, the patient could use a common information appliance like a cellular phone, frequently used to send and receive many kinds of data, thereby not revealing the medical application being used by the patient. While in private, the patient could use a proprietary medication dispenser with greater functionality, along with physiologic and blood analysis monitors, as these are clearly designed for use by someone with a chronic medical illness that the patient may not wish to disclose to others.

It is a further object of this invention that the patient-selected monitoring device will record information from the patient that signifies whether, and to what extent the patient is complying with the treatment plan; and communicate that information to a database. The database in turn is thereby updated, such that when the patient selects a different monitoring device, the database will send new and updated information to the second device, and not repeat the information sent to the first device, and already responded to by the patient.

It is a further object of this invention that updated information services be made available to the user, patient or caregiver over multiple devices available to each user of the information system. The information services include, but are not limited to; compliance monitoring, physiologic monitoring; laboratory test results; personal emergency response monitoring; patient counseling; patient performance reports that indicate how well the patient is following the medical treatment plan; patient peer-group-based performance/outcomes reports, drug-interaction reports (prescription, over-the-counter, herbal, nutriceutical, vitamin, etc.), and other reports. The database business logic and rules, along with formatting and communications software will determine for each device used by the user the appropriate time and formatting for presenting the information to that device; and for capturing information from each device. The informational reports that correspond to the patient's medication compliance and health status could be accessed from the database by the multiple devices via an Internet, Extranet, Intranet, direct dial-in, wireless broadcast, faxed, mailed, or telephoned means of communication. In this way each user can select the particular information device best suited to their particular needs at the time. These devices could include a laptop PC, desktop PC, Personal Digital Assistant (PDA), smart phone, or other computing device.

Each patient monitor may, but need not, have associated with it medication compartments that communicate with the monitor or clip onto it, such that the compartments have sensors that sense when the medication is being removed, and communicate this information to the memory of the device, or directly to the database.

Once the patient file is transmitted to these monitoring devices, the monitoring device firmware or software converts the file into a series of messages and queries to assist the patient in following the protocol, by prompting and monitoring a series of events throughout the day. An alternative would be for the database itself to convert the patient file containing the medical treatment plan into the prompting and monitoring events, and communicate these to the monitoring devices at the appropriate time. Another alternative would be for a wireless signal to carry the application software and the patient file for direct insertion into the wireless patient monitoring devices. The patient then interacts with the selected monitoring device, which in turn communicates the collected patient information back to the database for report-generation to the medical personnel, caregivers and family members who are monitoring and managing the patient. In this fashion a complex protocol that requires a sequential set of actions and monitoring activities can be managed via two or more devices, synchronously linked to a database, only one of which is used by the patient at a particular time.

The data elements that comprise the medical treatment plan entered into the database may relate to instructions or medical educational content about specific medication; medication dosage; patient physiological measurement, for example weight, blood pressure, pulse rate, glucose level, any antigen level, pH, pO₂, temperature, EKG rhythm, pO₂ saturation of the blood, hormone level; any psychological measurement, for example the score based upon standardized or non-standardized tests measuring anxiety, stress, anger, suicidal tendencies, schizophrenic relapse, rapid cycling bipolar relapse or confusion; medical education content related to any disease state or medical condition, for example congestive heart failure, hypertension, angina, circulatory inadequacy or other disease condition of the cardiopulmonary and circulatory systems, specific organ failure, dysfunction of an organ or system or transplanted organ such as asthma for the lungs, kidney failure for the kidney, arteriosclerosis or atherosclerosis of the heart, and transplantation of lung, kidney or heart; class of pathogen, or a specific pathogen; for example instructions or content may be based upon viral infection, or upon a specific viral disease such as HIV/AIDS, hepatitis A,B,C,D,E or G. Similarly, the instructions or content comprising the data elements may be based upon a bacterial disease agent, for example tuberculosis, malaria, cholera and meningitis; a specific microbial agent such as a virus, bacteria, mycotic infection and parasitic infection; or may be based upon the type of disease or pathology involved or the physiological system effected; for example cancer, autoimmune disease, muscular-skeletal system, or pathology of the hematopoetic, circulatory, reproductive, dermatologic, digestive, endocrine or nervous systems.

In each situation, the patients' medical protocols will be changed based upon the patients' condition, by entering into the individual patient file resident in the database the elements of a medical protocol specific to that patient; for example medication type; medication dosage; dietary regimen; specific reminders, such as when to obtain a medication refill, when to call the doctor; and any algorithm-driven events that are based upon data inputted by the patient into the remote monitoring device, for example instructing the patient to dial “911” if they are having chest pain and have taken too much bronchodilator medication.

The database software may be any functional database system, for example Oracle and Microsoft. The business logic rules may be any functional system, such as TEOCO Corporation's PageNgine, or DREAM (Dynamic Rapid Enterprise Application Method). The software that synchronizes the database with the remote patient monitoring devices may be any functional system, such as Aether Corporation's ScoutSync technology. The remote device may be any communications device such as a Personal Digital Assistant such as Palm Pilot®, cellular telephone, interactive pager, interactive television, or proprietary device such as the Med-eMonitor™.

The database application, in this case provided in a Microsoft SQL Server 7 database, can be converted into configuration files that are downloaded via modem, cable, infra-red, laser, computer disk, or wireless means into the multiple patient monitors used by each individual patient. The files are translated into routines that convert the medical protocol structure into a series of prompt and record events. In this way the patients get the benefit of treatment protocols on a real-time basis regardless of which monitor they use at a particular time.

Once the caregivers populate the data fields in the database by selecting a combination of data elements for the medical treatment plan from an infinite number of possible data elements, they enter the data elements into fields of the database. The database converts these fields into configuration files that are downloaded into the individual patient's devices or monitors and stored in their memories. In the alternative, the database may sequentially transmit the real-time prompt-and-record events directly into the patient devices. This method results in patient management protocols that can be instantaneously updated. Through this method, the caregivers need not develop new software each time they want to change the database or patient protocols; and the patient needn't worry that a particular monitor, unsuited for a particular lifestyle situation, need be used.

Alarm-based medication events, educational content messages, alarm-based treatment instructions, questionnaires, and any other elements contained in the medical treatment plan or protocol can be delivered over time and space to all of the remote monitoring devices in a synchronized fashion. As the patient responds to a particular device by inputting data into the device, the database synchronously updates its patient file and the treatment regimen.

The events that are then prompted and recorded can be organized and managed in an event log as illustrated below, which corresponds with the appropriate fields of the database that is prepared to accept the events from the event log. This event log listing is by no means meant to be limiting, as the event log could also include: specific medication; medication dosage; patient physiological measurement, for example weight, blood pressure, pulse rate, glucose level, any antigen level, pH, pO₂, temperature, EKG rhythm, pO2 saturation of the blood, hormone level; any psychological measurement, for example the score based upon standardized or non-standardized tests measuring anxiety, stress, anger, suicidal tendencies, schizophrenic relapse, rapid cycling bipolar relapse or confusion; medical education content related to any disease state or medical condition, for example congestive heart failure, hypertension, angina, circulatory inadequacy or other disease condition of the cardiopulmonary and circulatory systems, specific organ failure, dysfunction of an organ or system or transplanted organ such as asthma for the lungs, kidney failure for the kidney, arteriosclerosis or atherosclerosis of the heart, and transplantation of lung, kidney or heart; class of pathogen, or a specific pathogen; for example instructions or content may be based upon viral infection, or upon a specific viral disease such as HIV/AIDS, hepatitis A,B,C,D,E or G. Similarly, the instructions or content comprising the data elements may be based upon a bacterial disease agent, for example tuberculosis, malaria, cholera and meningitis; a specific microbial agent such as a virus, bacteria, mycotic infection and parasitic infection; or may be based upon the type of disease or pathology involved or the physiological system effected; for example cancer, autoimmune disease, muscular-skeletal system, or pathology of the hematopoetic, circulatory, reproductive, dermatologic, digestive, endocrine or nervous systems.

Any monitoring device capable of communicating by modem, fax, phone line or wireless means may be used to collect the patient data that is communicated to the database for storage and for display. Among the monitoring devices is the Medi-Monitor® described in PCT patent application WO98/38909 herein incorporated by reference. The database containing the patient information and protocols is the site of the medical treatment plans made by the methods of the present invention. The database incorporating the treatment plan then uploads the modified plan to each patient's monitoring devices for use by the patient as directed by the real-time, self-selected, synchronized, database-linked method.

For example, using the above method, a complex treatment regimen for a heart failure patient may be created, converted into a series of prompt-and-record events to be delivered to multiple patient monitors used by the single patient. Heart failure patients may typically require the following data elements in their medical treatment plan, with information about each data element to be communicated at a particular time: a medication regimen comprised of a beta-blocker, ACE inhibitor, diuretic, and Digoxin; dosing instructions for each medication; a description of each medication and rationale for taking the medication; what the medication looks like along with its color; daily measures of weight, blood pressure, pulse, and blood oxygen levels; a salt restricted, low fat, low calorie diet; an exercise program tailored to their specific level of heart failure; questionnaires that assess their shortness of breath, chest pain, energy level, and swelling of the legs; information updates about new drugs used to treat the condition; information about when to call the doctor with specific complaints or side effects or adverse drug reactions; and other information.

Using the above method, this highly complicated treatment plan can be converted into fields in the database, then into a series of real-time messages and recorded events that are communicated to the patient via the remote devices, whereby the patient interacts with the devices to respond to the messages and answer the queries; and whereby the patient uses a variety of physiological monitors, and whereby the remote devices then communicate the patient responses to the database.

E. Pharmaco-Economic Analysis

In an embodiment of the present invention, system 100 provides self-selected, synchronized, database-linked medical monitors to outpatients and their caregivers preferably, but not necessarily, as described in commonly assigned U.S. patent Ser. No. 60/226,515, filed Aug. 21, 2000, (herein referred to as the '515 application). The disclosure of the '515 application is incorporated herein by reference as though set forth in its entirety.

The present invention describes methods and apparatus useful in analyzing the dose-specific effects of pharmaceutical agents upon the patient or populations of patients, based upon statistical analysis and data mining of data contained in a medical database. The medical database may contain the following real-time data captured from patients: medication compliance for one or more medications taken by the patient, patient answers to health status and quality-of-life questionnaires, patient physiologic data, and patient laboratory data. This data may also be combined with other database data such as genomic, proteomic, phenotypic, economic, and other healthcare related data, for further data analysis. The statistical analysis and data mining of the data includes the method of correlating patient medication dosing patterns of one or more drugs ingested by the patient, with various other measures of clinical and economic outcomes of the patients. The method is based in translating a complex medical treatment plan of a medical outpatient into a sequential series of automated, prompt and record events presented over time; to two or more medical monitors used by a single patient. The medical monitors capture data on medication compliance, health status, quality-of-life, physiologic status (e.g. blood pressure, EKG, pO2, pulse rate, weight, pulmonary function, etc.), and various measures of blood, serum, urine, and other laboratory tests. The medical monitors are linked to a database that updates and synchronizes the information presented to all of the monitors, and captures information from all of the monitors. Individual patient medical treatment plans may be remotely created, modified, or viewed in the database; and in turn are transformed by business logic rules and a communications system into prompted and recorded events that are delivered to the patient by the remote monitors. The patient monitoring data may then be combined with genomic, proteomic, and phsyiologic data for analysis and data mining purposes. In addition, the method provides for each device to record whether the patient is following the treatment plan; and to upload that recorded data to the database; which provides for monitoring the progress of treatment as the individual patient uses multiple monitoring devices. The monitored outpatient may be managed by one or more medical treatment or clinical research protocols or plans that involve; pharmaceutical drugs, physiologic data, treatment instructions, medical educational content, medication compliance assessment, and health status or quality of life assessment. Each of these protocols or plans is first translated into a set of data points stored in a medical database and configured as a patient medical file. The data points are then translated into a sequence of time-and-event-driven, graphic and/or auditory, real-time communications presented to the patient or caregiver via remote devices that are linked to the database. The patient then interacts with remote devices that record these interactions in real-time, and communicate the recorded data to the database in real-time or via store-and-forward means. By creating medical databases containing data points that define the outpatient's medical treatment plan or protocol; and linking them with real-time patient monitoring devices with different monitoring capabilities that can record the patient's responses; the patient data thereby derived can be analyzed and mined; or combined with genetic, proteomic, and phenotypic data for further analysis; to determine the relationship between drug doses, patient outcomes, and costs of treatment. The method has application in clinical drug trials and outcomes research protocols, which are designed to determine the therapeutic effects, side effects, adverse drug events, and costs of new pharmaceutical agents, and agents that are already on the market. It also has applications in targeting specific drugs at specific doses for specific patient populations, to optimize patient outcomes and minimize side effects, adverse drug events, and costs.

Medication is widely regarded as the most cost-effective medical means of treating patients. Prior to and subsequent to bringing a medication onto the market, the pharmaceutical manufacturer must subject the drug compound to rigorous pre-clinical and clinical tests, to determine its safety, efficacy, and costs. These tests are conducted in clinical drug trials, wherein the compound is first tested in animals, then small populations of humans, then in larger populations of humans. First the safety (incidence and severity of side effects) of the drug must be established. Then preliminary data on effectiveness (efficacy) must be established. These early safety and efficacy trials are conducted in small numbers of human study participants. Then larger trials, at times at multiple different pharmaceutical dosing levels, further define the safety and efficacy profile for the drug compound at particular doses. The average cost of bringing anew drug to market is $500,000,000, and takes an average of seven years. Each day of delay in bringing the drug onto the market costs the manufacturer $1,000,000 to $10,000,000 in lost sales, and reduces the post-market life of the drug's patent. Once the drug is on the market, post-market surveillance studies are used to better understand safety, efficacy, the prevalence of side effects, and drug interactions with other drug compounds in combination with the new drug being studied. In addition, new techniques of analyzing the human genome (genomics), human protein structure and function (proteomics), and the physical expression of the genes (phenotyping) are being used to help determine the different responses of genetically and phenotypically different populations of patients to pharmaceutical agents. Various methods may be used to analyze the effectiveness, cost, and adverse effects of medication. Currently these methods are very labor intensive, and have limited technology integrated into the clinical drug trial system. Many of the systems are paper-based, and rely on retrospective data and written patient diaries (Source: “ePharma, Accelerating Clinical Trials and Enhancing Details,” Friedman Billings Ramsey Healthcare Industry Analysis, August 2000).

Recently, various technologies have been introduced into the clinical drug trial process, including electronic devices that may be used to monitor and manage medical treatment regimens and protocols for treating a patient's medical condition and monitoring the patient's outcomes. These devices communicate one or more of the following: medication schedule and instruction data, medical treatment data, medical education content, patient query data and patient response data, and physiologic data. The devices include a controller for controlling modes of device operation, controlling access to the memory, controlling the communication of treatment data and patient query data on a display or via voice communications means, receiving and processing patient response data, tracking timing, and providing scheduled medication alarm signals. The devices may include soft function keys interfaced with the controller. The soft function keys signal the controller, commanding it to execute different modes of operation of the medical monitoring device. The devices may also provide for scheduled medication alarm signals that alert the user concerning prescribed medications to be taken.

However, what is missing from current methods of pharmacoeconomic analysis in clinical drug trials is a medical database that contains the following real-time data captured from patients: medication compliance for one or more medications taken by the patient, patient answers to health status and quality-of-life questionnaires, patient physiologic data, and patient laboratory data.

What is also missing is the ability to combine this data with other database data such as genomic, proteomic, phenotypic, economic, and other healthcare related data, for further data analysis.

What is also missing is the statistical analysis and data mining of part or all of the data to include the method of correlating patient medication dosing patterns of one or more drugs ingested by the patient, with various other measures of clinical and economic outcomes of the patients, by translating a complex medical treatment plan of a medical outpatient into a sequential series of automated, prompt and record events presented over time; to medical monitors used by a single patient. The medical monitors capture data on medication compliance, health status, quality-of-life, physiologic status (e.g. blood pressure, EKG, pO2, pulse rate, weight, pulmonary function, etc.), and various measures of blood, serum, urine, and other laboratory tests. The medical monitors are linked to a database that updates and synchronizes the information presented to all of the monitors, and captures information from all of the monitors. Individual patient medical treatment plans may be remotely created, modified, or viewed in the database; and in turn are transformed by business logic rules and a communications system into prompted and recorded events that are delivered to the patient by the remote monitors.

What is also missing form the present methods is the ability to combine the patient monitoring data with genomic, proteomic, and physiologic data for analysis and data mining purposes, to predict the responses of subpopulations of patients to a given drug or combination of drugs, in different doses and combinations, based upon the specific genetic and physical attributes of these subpopulations.

It is therefore an object of the present invention to enter into a medical database the following real-time data captured from patients: medication compliance for one or more medications taken by the patient, patient answers to health status and quality-of-life questionnaires, patient physiologic data, and patient laboratory data.

It is a further object of the present invention to provide the ability to combine this data with other database data such as genomic, proteomic, phenotypic, economic, and other healthcare related data, for further data analysis.

It is a further object of the present invention that the statistical analysis and data mining of part or all of the data will include the method of correlating patient medication dosing patterns of one or more drugs ingested by the patient, with various other measures of clinical and economic outcomes of the patients, by translating a complex medical treatment plan of a medical outpatient into a sequential series of automated, prompt and record events presented over time; to medical monitors used by a single patient. The medical monitors capture data on medication compliance, health status, quality-of-life, physiologic status (e.g. blood pressure, EKG, pO2, pulse rate, weight, pulmonary function, etc.), and various measures of blood, serum, urine, and other laboratory tests.

It is a further object of the present invention that combination of the patient monitoring data with genomic, proteomic, and physiologic data for analysis and data mining purposes will predict the responses of subpopulations of patients to a given drug or combination of drugs, in different doses and combinations, based upon the specific genetic and physical attributes of these subpopulations.

It is a further object of the present invention that the knowledge thereby gained from the data analysis and mining can be used to: adjust the dosing recommendations of one or more medications to improve clinical outcome, reduce the frequency of side-effects, reduce the severity of side-effects, eliminate adverse drug events, reduce or eliminate drug-interactions, and determine the most cost-effective means of using drugs to improve the health of specific populations of patients.

The data elements that comprise the medical data entered into the database may relate to instructions or medical educational content about specific medication; medication dosage; patient physiological measurement, for example weight, blood pressure, pulse rate, glucose level, any antigen level, pH, pO2, temperature, EKG rhythm, pO2 saturation of the blood, hormone level; cell surface receptors; serum proteins; DNA data; Protein data; any psychological measurement, for example the score based upon standardized or non-standardized tests measuring anxiety, stress, anger, suicidal tendencies, schizophrenic relapse, rapid cycling bipolar relapse or confusion; medical education content related to any disease state or medical condition, for example congestive heart failure, hypertension, angina, circulatory inadequacy or other disease condition of the cardiopulmonary and circulatory systems, specific organ failure, dysfunction of an organ or system or transplanted organ such as asthma for the lungs, kidney failure for the kidney, arteriosclerosis or atherosclerosis of the heart, and transplantation of lung, kidney or heart; class of pathogen, or a specific pathogen; for example instructions or content may be based upon viral infection, or upon a specific viral disease such as HIV/AIDS, hepatitis A,B,C,D,E or G. Similarly, the instructions or content comprising the data elements may be based upon a bacterial disease agent, for example tuberculosis, malaria, cholera and meningitis; a specific microbial agent such as a virus, bacteria, mycotic infection and parasitic infection; or may be based upon the type of disease or pathology involved or the physiological system effected; for example cancer, autoimmune disease, muscular-skeletal system, or pathology of the hematopoetic, circulatory, reproductive, dermatologic, digestive, endocrine or nervous systems.

The database software may be any functional database system, for example Oracle and Microsoft. The DNA profile detection may be provided by any DNA Chip System, such as that provided by Affymatrix Corporation. The serum protein and cell surface receptor data may be provided by any Protein Chip System, such as that provided by Affymatrix Corporation. The medical data statistical analysis and data mining functions can be performed by any functional system, such as that provided by Medical Internet Solutions, Inc. The business logic rules may be any functional system, such as TEOCO Corporation's PageNgine, or DREAM (Dynamic Rapid Enterprise Application Method). The software that synchronizes the database with the remote patient monitoring devices may be any functional system, such as Aether Corporation's ScoutSync technology. The remote device may be any communications device such as a Personal Digital Assistant such as Palm Pilot®, cellular telephone, interactive pager, interactive television, or proprietary device such as the Med-eMonitor™.

The medical database used to store and analyze the following real-time data captured from patients; medication compliance for one or more medications taken by the patient, patient answers to health status and quality-of-life questionnaires, patient physiologic data, patient laboratory data, genomic, proteomic, phenotypic, economic, and other healthcare related data can be accomplished utilizing a standard ODBC (Object-oriented database compliant database). The example shown below, written in Microsoft SQL Server 7, is illustrative, and should in no way be construed as limiting the invention.

The database application, in this case provided in a Microsoft SQL Server 7 database, can be converted into configuration files that are downloaded via modem, cable, infra-red, laser, computer disk, or wireless means into the multiple patient monitors used by each individual patient. The files are translated into routines that convert the medical protocol structure into a series of prompt and record events. In this way the patients get the benefit of treatment protocols on a real-time basis regardless of which monitor they use at a particular time.

Once the caregivers populate the data fields in the database by selecting a combination of data elements for the medical treatment plan from an infinite number of possible data elements, they enter the data elements into fields of the database. The database converts these fields into configuration files that are downloaded into the individual patient's devices or monitors and stored in their memories. In the alternative, the database may sequentially transmit the real-time prompt-and-record events directly into the patient devices. This method results in patient management protocols that can be instantaneously updated. Through this method, the caregivers need not develop new software each time they want to change the database or patient protocols; and the patient needn't worry that a particular monitor, unsuited for a particular lifestyle situation, need be used.

Alarm-based medication events, educational content messages, alarm-based treatment instructions, questionnaires, and any other elements contained in the medical treatment plan or protocol can be delivered over time and space to all of the remote monitoring devices in a synchronized fashion. As the patient responds to a particular device by inputting data into the device, the database synchronously updates its patient file and the treatment regimen.

The events that are then prompted and recorded can be organized and managed in an event log as illustrated below, which corresponds with the appropriate fields of the database that is prepared to accept the events from the event log. This event log listing is by no means meant to be limiting, as the event log could also include: specific medication; medication dosage; patient physiological measurement, for example weight, blood pressure, pulse rate, glucose level, any antigen level, pH, pO₂, temperature, EKG rhythm, pO2 saturation of the blood, hormone level; any psychological measurement, for example the score based upon standardized or non-standardized tests measuring anxiety, stress, anger, suicidal tendencies, schizophrenic relapse, rapid cycling bipolar relapse or confusion; medical education content related to any disease state or medical condition, for example congestive heart failure, hypertension, angina, circulatory inadequacy or other disease condition of the cardiopulmonary and circulatory systems, specific organ failure, dysfunction of an organ or system or transplanted organ such as asthma for the lungs, kidney failure for the kidney, arteriosclerosis or atherosclerosis of the heart, and transplantation of lung, kidney or heart; class of pathogen, or a specific pathogen; for example instructions or content may be based upon viral infection, or upon a specific viral disease such as HIV/AIDS, hepatitis A,B,C,D,E or G. Similarly, the instructions or content comprising the data elements may be based upon a bacterial disease agent, for example tuberculosis, malaria, cholera and meningitis; a specific microbial agent such as a virus, bacteria, mycotic infection and parasitic infection; or may be based upon the type of disease or pathology involved or the physiological system effected; for example cancer, autoimmune disease, muscular-skeletal system, or pathology of the hematopoetic, circulatory, reproductive, dermatologic, digestive, endocrine or nervous systems.

Any monitoring device capable of communicating by modem, fax, phone line or wireless means may be used to collect the patient data that is communicated to the database for storage and for display. Among the monitoring devices is the Medi-Monitor® described in PCT patent application WO98/38909 herein incorporated by reference. The database containing the patient information and protocols is the site of the medical and economic data made by the methods of the present invention.

For example, using the above method, a complex treatment regimen for a heart failure patients of a specific age, gender, race, and phenotype may be created, and converted into a series of prompt-and-record events to be delivered to multiple patients used by patients. Heart failure patients may typically require the following data elements in their medical treatment plan, with information about each data element to be communicated at a particular time: a medication regimen comprised of a beta-blocker, ACE inhibitor, diuretic, and Digoxin; dosing instructions for each medication; a description of each medication and rationale for taking the medication; what the medication looks like along with its color; daily measures of weight, blood pressure, pulse, and blood oxygen levels; a salt restricted, low fat, low calorie diet; an exercise program tailored to their specific level of heart failure; questionnaires that assess their shortness of breath, chest pain, energy level, and swelling of the legs; information updates about new drugs used to treat the condition; information about when to call the doctor with specific complaints or side effects or adverse drug reactions; and other information. The current method will insure that the exact regimen is prescribed as a part of the medical treatment plan for a subpopulation of patients, such that the optimum doses of the optimum combinations of medications, at the least cost, will produce the best outcomes.

F. Creation and Self-Selection of Musical Alarms

In an embodiment of the present invention, system 100 is configurable to create and select musical alarms preferably, but not necessarily, as described in commonly assigned U.S. patent Ser. No. 60/228,360, filed Aug. 28, 2000, (herein referred to as the '360 application). The disclosure of the '360 application is incorporated herein by reference as though set forth in its entirety.

The present invention describes methods and apparatus useful in remotely modifying a medical treatment protocol, to improve a patient's adherence to his or her medication regimen and medical treatment protocol through musical alarms. Individual patient medical treatment protocols may be remotely created and modified, and may include specific musical compositions that are audibly presented to the patient at pre-selected times over one or more medical monitoring devices used by the patient. An audible musical alarm is presented to the patient in association with a prompted event, as part of an information system designed to prompt the patient to follow particular elements of the medical treatment plan. An audible musical alarm is presented with each of the following events: “Alarm” Event, which is the sound made when there is an event prompted for the taking of medication, answering a questionnaire, or another instruction, such as the use of a physiological monitoring device; “KeyClick” Event, which is the sound made when a button is pressed on a medical monitoring device at an acceptable time; “BadKeyClick” Event which is the sound made when a button is pressed on the medical monitoring device at an unscheduled time; “DrawerOpenGood” Event, which is the sound made when a medication drawer is opened at a scheduled time; “DrawerOpenBad” Event, which is the sound made when a medication drawer is opened at an unscheduled time; “DrawerClose” Event, which is the sound made whenever a medication drawer is closed; and other event-associated sounds related to prompting the patient to follow his or her medical treatment plan. Each musical alarm can be self-selected by the patient from a menu of possible selections, based upon the musical alarm that is most desired by, and most motivating for the patient to follow the medical treatment plan. In addition, the present invention provides for the patient to be able to compose one or more musical selections, each of which to be presented in association with one or more medical treatment events. In addition the present invention provides a method to download a musical composition from a database or the Internet, and convert this music to musical alarms presented in association with one or more medical treatment events. In addition, the present invention provides for a means of data analysis, to determine which musical composition or compositions are most likely to promote adherence to a medication regimen and medical treatment plan by a specific patient or group of patients. Pattern recognition and data analysis software provides for the ability to analyze which musical alarm prompts promote the best patient medication compliance and medical outcomes for a particular subgroup of patients, whereby a mass customization medical database feature then enables the assignment of the most effective musical alarms to optimize the outcomes of specific patient subgroups. The method has application in medical databases containing the data collected by remote devices that monitor the medication compliance and health outcomes of outpatients. These outpatients may be managed by one or more medical treatment plans or clinical research protocols involving pharmaceutical drugs, physiological data, educational content, and health status assessment or quality of life questionnaires. The medical protocols may be mass customized, and reside in a medical database, and be useful in efficiently monitoring and managing medical outpatients; and in addition the method provides for specific patient instructions for the individualized components of the medical protocols. By using the creation and self-selection of musical alarms method, patient adherence to medication regimens and medical treatment plans will be enhanced, and individual patient management goals may be better realized.

Various electronic devices may be used to monitor and manage medical treatment regimens and protocols for treating a patient's medical condition. These devices store one or more of the following: medication schedule data, treatment data, medical education content, patient query data and patient response data, including physiologic response. The devices include a controller for controlling modes of device operation, controlling access to the memory, controlling display treatment data and the patient query data on a display, receiving and processing patient response data, tracking timing, and providing scheduled medication alarm signals and other types of alarm signals to prompt events related to the medical treatment regimen. The devices may include two or more soft function keys interfaced with the controller. The soft function keys signal the controller, commanding it to execute different modes of operation of the medical monitoring device. The devices may also provide for scheduled medication alarm signals that alert the user concerning prescribed medications due to be taken.

While using various devices to monitor outpatients with chronic illnesses, and among clinical drug trial participants, patients often face complicated and burdensome medical treatment protocols or regimens. These protocols require the patient to carry out a detailed series of events, in a sequential fashion throughout the day, related to taking medication, following other instructions (e.g. taking their blood pressure or blood glucose levels and transmitting these values to a remote database), answering questions that assess their health status, and other events that need to be prompted and recorded. Some of these events and information are population-specific, and apply to entire populations of patients with specific diseases. Other events and information apply to sub-populations of patients, and still other events are specific to the individual patients themselves and represent specific elements of the protocol that apply only to them and to no other patient. These events may be prompted by a series of audible alarms presented to the patient. It is well known that certain musical sounds are likely to produce certain types of behaviors. Certain “Music Soothes the Savage Beast,” while other music can promote negative or aggressive feelings. Certain patients, for example those suffering from schizophrenia, are acutely sensitive to noise, and may refrain from using medical monitoring devices solely because the alarm sounds from the devices are offensive to the patients and/or their caregivers (personal communication from Dr. Paul Ruskin, Principal Investigator at the Baltimore VA Hospital Schizophremia Research Project using the Med-eMonitor System).

What is missing from the previous methods, yet desirable for the medical personnel, caregivers, or family members monitoring and managing outpatients, is the ability to readily and easily select those audible alarms that are most appealing to the patient, and most likely to facilitate the patient's adherence to his or her medication regimen and medical treatment plan. Some patients may prefer a soothing classical composition, while others may prefer a popular and lively tune. Others may prefer the musical note chimes of Big Ben, that characterize many church bell chimes. Still others may prefer to select a somber composition to coincide with a warning message that indicates they are not properly following the medication regimen or treatment plan. In addition, analysis of patient outcome data may provide information that correlates one or more musical compositions with positive or negative patient outcomes. Once this correlation is determined, the mass customized database described by the present invention would enable specific subgroups of patients and those who monitor them to insure that the subgroups of patients get the right musical alarms at the right time, to optimize the patient's following a complex medical protocol.

It is therefore the object of the present invention to enable those patients, medical personnel and caring family members to select and provide specific musical alarms to individual patients and to populations of patients.

It is the further object of the present invention that the selection of the musical alarms is made from a list of possible compositions, which in turn would automatically be played by the medical monitoring devices used by the patients, at pre-selected event times.

It is the further object of the present invention that the musical alarms could be composed by the patient, medical personnel, and caring family members, by creating the specific frequencies and duration of the musical notes that comprise the musical alarms in the patient's file contained in the medical database. These musical alarms in turn would be audibly presented to the patients by the medical monitoring devices, or other devices, at specific event times related to the patients' medical treatment plans.

It is the further object of the present invention that the musical alarms could be created by downloading music from a database or the Internet, and converting the downloaded music into the correct frequencies and duration of musical notes to be presented to the patient over the medical monitoring device.

It is the further object of the present invention to determine the correlation of specific musical alarms with patient adherence to medication regimens and medical treatment plans, such that specific musical alarms are determined to optimize outcomes of treatment for particular individual patients or populations or patients.

It is the further object of the present invention that mass assignment of specific musical alarm compositions to specific populations of patients be enabled by a remotely accessible database that contains the patient files. Each patient file is constructed in such a fashion that it may be assigned population-specific musical alarms, determined to be of value to the management or monitoring of the patient or population of patients.

It is the further object of the present invention that the musical alarms be presented to the patients and their caregivers over one or more medical monitoring devices.

Each monitor may, but need not, have associated with it medication compartments that communicate with the monitor or clip onto it, such that the compartments have sensors that sense when the medication is being removed, and communicate this information to the memory of the device, or directly to the database.

Once the patient file is transmitted to these monitoring devices, the monitoring device firmware converts the file into a series of messages and queries to assist the patient in following the protocol, by prompting and monitoring a series of events throughout the day. The prompted events may be associated with musical alarms that are audibly presented at the time of the events. In the alternative, a wireless signal can carry the application software and the patient file for direct insertion into the wireless device. The patient then interacts with the monitoring devices, which in turn communicate the collected patient information back to the database for report-generation to the medical personnel, caregivers and family members who are monitoring and managing the patients. New individual patient assignment or mass customization of the musical alarms contained in the patient files can then take place, based upon the collected data from the patient which is correlated with specific musical alarms that were played to the patient. In this fashion a complex protocol that requires a sequential set of actions and monitoring activities can be optimized by prompting the actions through presenting musical alarms associated with messages that define the protocol.

The sub-populations or groups of patients that receive specific musical alarms to optimize their medical treatment and medication adherence outcomes may be any group of patients that share one or more common characteristic that may effect or modify their medical condition or treatment protocols. Among the groups to consider are defined by age; gender; occupation; disease state; medical history event; medication category; specific medication; medication dosage; patient physiological measurement, for example weight, blood pressure, pulse rate, glucose level, any antigen level, pH, pO₂, temperature, EKG rhythm, pO2 saturation of the blood, hormone level; or any psychological measurement, for example the score based upon standardized or non-standardized tests measuring anxiety, stress, anger, suicidal tendencies, schizophrenic relapse, rapid cycling bipolar relapse or confusion. The groups may be age, gender, race, national origin, geographic location related in combination with a medical condition. The group may be based upon the same or similar disease state or medical condition, for example congestive heart failure, hypertension, angina, circulatory inadequacy or other disease condition of the cardiopulmonary and circulatory systems. The group may be based upon specific organ failure or dysfunction or upon a transplanted organ, for example asthma for the lungs, kidney failure for the kidney, arteriosclerosis or atherosclerosis of the heart, and transplantation of lung, kidney or heart. The group may be passed upon class of pathogen, or a specific pathogen. For example the group may be based upon viral infection, or upon a specific viral disease such as HIV/AIDS, hepatitis A,B,C,D,E or G. Similarly, the group may be based upon a bacterial disease agent, for example tuberculosis, malaria, cholera and meningitis. Groups may be defined by a specific microbial agent such as a virus, bacteria, mycotic infection and parasitic infection. The group may be based upon the type of pathology involved or the physiological system effected; for example cancer, autoimmune disease, muscular-skeletal system, or pathology of the hemopoetic, circulatory, reproductive, digestive, endocrine or nervous systems.

The groups may be based upon two or more parameters, for example HIV/AIDS and immune function, liver dysfunction and hepatitis, or bone degeneration and arthritis. The groups may be based upon any patient parameter available to the medical database, either directly in the database or accessible through linkage to another database or databases, and associated with a specific medical condition. Among the data in the database are name of patient's physician, physician specialty, hospital, insurance company, type of insurance, diagnosis, length of illness, family history or previous medical history. The group may be based upon the type of clinical setting, for example hospital, emergency clinics, physician's office, institutional setting or home. The group may be based upon the name of the corporation, organization, physician, clinical research organization, pharmaceutical company, case worker or sponsor.

In each situation, the musical alarms in the patients' medical protocols will be changed based upon the patients' inclusion in such a group. The change in the musical alarm submitted to the group database files will modify each patient's medical protocol in the same manner. Each individual patient may be a member of many different sub-groups, each of which may have a change entered into the database. The changed patient protocol is then uploaded to the monitoring device. Such uploading may be immediate, or it may be at the next scheduled uploading event. Notice of the uploading of a change is made through the monitoring device to alert the patient to the change in protocol.

In addition each individual patient's musical alarm protocol may be modified in the database by entering into the individual patient configuration file the elements of the musical alarm specific to that patient; through entering the frequency and duration of each of the musical notes; or by selecting a musical alarm composition from a list of possible compositions; and then entering these musical alarms into the appropriate fields of the patient's configuration file as shown below.

The database software may be any functional system, for example Oracle and Microsoft.

The mass customization of the patient files, which are then translated into operating routines in remote monitors, can be accomplished utilizing a standard ODBC compliant database. The example shown below, written in Microsoft SQL Server 7, is illustrative, and should in no way be construed as limiting the invention.

As illustrated in the above database entry fields and configuration file structure, the goal of mass customization of patient protocols in clinical drug trials and in outpatient medical management is facilitated. The addition of pull-down menus to the fields, which enable the selection and population of the fields with standardized musical compositions, questionnaires and educational content, further simplifies the mass customization method. Other fields are then individually populated with patient-specific content to complete the patient's medical protocol design.

The database application of the mass customization system, in this case provided in a Microsoft SQL Server 7 database, can be converted into configuration files that are downloaded via modem, cable, or wireless means into the patient monitors. The files are translated into routines that convert the medical protocol structure into a series of prompted and recorded events. An example of this method is contained in the Configuration File Document above, which describes how the firmware resident in the medical monitoring device operates. In this way the patients get the benefit of both population-based as well as individualized assessment and/or treatment protocols on a real-time basis.

Once the caregivers populate the data fields in the database, which converts these fields into configuration files that are downloaded into the monitors, the patient medical management protocols can be instantaneously updated. Through this method, the caregivers need not develop new software each time they want to change the database or patient protocols (or create a musical composition), and can rapidly take advantage of newly discovered population-based medical evidence to be communicated to patients, to give patients the advantages of up-to-the-minute medical knowledge without any delay caused by reprogramming or creating new software.

Musical alarm-based educational content messages and instructions, musical alarm-based treatment instructions, and musical alarm-based questionnaires can be assigned to specific groups of patients as previously disclosed. In addition, each individual patient file can be programmed for specific medication instructions by populating the appropriate fields.

The musical notes used in the monitor may be from any source. They may be from 2-30 musical notes, or more preferably from 3-20 musical notes. For example these musical notes may be selected from recorded live music, from music synthesizers, from recorded music; or alternatively they may be downloaded from various Internet sites, such as those in MP3 format found by searching the Internet for mp3. An example of an Internet site with various music note collections may be found at napster.com hereby incorporated by reference.

An example of the musical alarms that can be created is listed in Table 1, with the associated frequencies and duration of each tone to be entered into the appropriate fields of the database, which in turn will be audibly presented to the patients via the medical monitoring devices:

TABLE 1

| | Freq | Dur | Freq | Dur | Freq | Dur | Freq | Dur | Freq | Dur | Freq | Dur | Freq | Dur | Freq | Dur | Freq | Dur | Freq | Dur |
|---|---|---|---|---|---|---|---|---|---|---|---|---|---|---|---|---|---|---|---|---|
| Mus- | 294 | 833 | 330 | 417 | 262 | 417 | 294 | 833 | 392 | 417 | 440 | 417 | 494 | 833 | 440 | 417 | 370 | 417 | 392 | 166 |
| ette | | | | | | | | | | | | | | | | | | | | 6 |
| by | | | | | | | | | | | | | | | | | | | | |
| J. S. | | | | | | | | | | | | | | | | | | | | |
| Bach | | | | | | | | | | | | | | | | | | | | |
| Min- | 392 | 833 | 262 | 417 | 294 | 417 | 330 | 417 | 349 | 417 | 392 | 833 | 262 | 833 | 262 | 833 | | | | |
| uet | | | | | | | | | | | | | | | | | | | | |
| by | | | | | | | | | | | | | | | | | | | | |
| J. S. | | | | | | | | | | | | | | | | | | | | |
| Bach | | | | | | | | | | | | | | | | | | | | |
| Sona- | 659 | 833 | 784 | 417 | 659 | 417 | 523 | 833 | 523 | 833 | 587 | 833 | 587 | 417 | 659 | 417 | 523 | 166 | | |
| tina | | | | | | | | | | | | | | | | | | 6 | | |
| by | | | | | | | | | | | | | | | | | | | | |
| Jakob | | | | | | | | | | | | | | | | | | | | |
| Sch- | | | | | | | | | | | | | | | | | | | | |
| mitt | | | | | | | | | | | | | | | | | | | | |
| And- | 587 | 417 | 784 | 417 | 587 | 417 | 784 | 417 | 659 | 417 | 523 | 417 | 587 | 833 | 523 | 166 | | | | |
| atino | | | | | | | | | | | | | | | | 6 | | | | |
| by | | | | | | | | | | | | | | | | | | | | |
| Igor | | | | | | | | | | | | | | | | | | | | |
| Stra- | | | | | | | | | | | | | | | | | | | | |
| vins- | | | | | | | | | | | | | | | | | | | | |
| ky | | | | | | | | | | | | | | | | | | | | |
| Big | 440 | 833 | 349 | 833 | 392 | 833 | 262 | 249 | 262 | 833 | 392 | 833 | 440 | 833 | 349 | 249 | | | | |
| Ben's | | | | | | | | 9 | | | | | | | | 9 | | | | |
| Chi- | | | | | | | | | | | | | | | | | | | | |
| mes | | | | | | | | | | | | | | | | | | | | |

1. Frequencies (“Freq”) are calculated based upon an Octave Table found in “Reblitz”, Arthur A., “Piano Servicing, Tuning and Rebuilding”-“For The Professional, The Student, The Hobbyist,” Second Edition, The Vestal Press Limited, 1993, page 211.
2. Durations (“Dur”) are calculated by converting a metronome quarter note set at “72” into corresponding milliseconds by dividing 60 seconds per minute by 72, resulting in 833 milliseconds per quarter note.

Thus a creation and self-selection of musical alarms to optimize adherence to medication regimens and medical treatment plans, along with mass customization of patient management protocols, is facilitated by the above database design; and implemented by downloading into monitors that translate the database data into prompted and recorded events that are presented in association with the musical alarms.

Any monitoring device capable of communicating by modem, fax, phone line or wireless means may be used to collect the patient data that is communicated to the database for storage and for display; and for presenting the patient a series of events along with the audible presentation of musical alarms. Among the monitoring devices is the Medi-Monitor® described in PCT patent application WO98/38909 herein incorporated by reference. The database containing the patient information and protocols is the site of the group musical alarm changes made by the methods of the present invention. The database incorporating the group changes then uploads the modified protocol to each patient's monitoring device for use by the patient as directed.

The database software may be any functional database system, for example Oracle and Microsoft. The Business Logic Rules portion of the database may be created by applications written for a variety of commercially available software products, for example the PageNgine by TEOCO Corporation. The software that translates downloaded music from a database or the Internet, and converts it to the correct frequencies and duration of musical notes to be presented to the patient, may be any application written to analyze the frequency and duration of each tone of the downloaded music, and transferring these frequencies and durations to the appropriate fields of the database. The risk analysis software, that analyzes the medical risks posed by different patients or populations of patients, for example, could be an application written for the MEDSTAT Corporation Disease Staging Software. The decision support software for medical interventions could be an application written for the Apache Medical Systems Voyager+ Software. The software that enables new pattern recognition among data elements; and enables the caregiver or family member to analyze the correlation between musical alarms and patient outcomes, and then select the musical alarm compositions that determine optimized patient outcomes, could be an application written for Synera Corporation Discovery Software. The broadcast software could be an application written for the Mobile Application Platform developed by Phone2 Networks, Inc. The remote devices may be any communications devices such as a Personal Digital Assistant such as Palm Pilot®, cellular telephone, interactive pager, interactive television, or proprietary device such as the Med-eMonitor™.

G. Streaming Video and Pictorial Representation

In an embodiment of the present invention, system 100 streams video and pictorial representation to monitor and enhance medication compliance, physiologic status, health status, and pharmaceutical sales preferably, but not necessarily, as described in commonly assigned U.S. patent Ser. No. 60/230,367, filed Sep. 6, 2000, (herein referred to as the '367 application). The disclosure of the '367 application is incorporated herein by reference as though set forth in its entirety.

The present invention describes methods and apparatus useful in analyzing the effects of streaming video and pictorial presentations upon the patient or populations of patients, and upon the prescribing physicians; based upon statistical analysis and data mining of data contained in a medical database, physician prescribing patterns, and pharmaceutical sales data. The medical database may contain the following real-time data captured from patients: medication compliance for one or more medications taken by the patient, patient answers to health status and quality-of-life questionnaires, patient physiologic data, and patient laboratory data. This data may also be combined with other database data such as physician prescribing patterns, medication sales, genomic, proteomic, phenotypic, economic, and other healthcare related data, for further data analysis. The statistical analysis and data mining includes the method of correlating one or more streaming video presentations and/or audiovisual and/or visually-presented advertisements independently presented to patients and physicians with the following patient-related data; medication dosing patterns of one or more ingested drugs, various other measures of clinical and economic outcomes, prescribing patterns of the treating physicians, and sales of the medications. The method may based in translating a complex medical treatment plan of a medical outpatient into a sequential series of automated, prompt and record events presented over time; to medical monitors or other data capture devices used by patients. These presented events may include selections of streaming video content and/or banner advertisements that are directed to specific patients or populations of patients, and directed to specific physicians or specialty groups of physicians, based upon respective, pre-defined patient and physician characteristics. The physicians are presented the advertisements via their personal computers (PCs), personal digital assistants (PDAs), interactive televisions, Palm Pilots®, or other technologies. The patient's medical monitors capture data on medication compliance, health status, quality-of-life, physiologic status (e.g. blood pressure, EKG, pO2, pulse rate, weight, pulmonary function, etc.), and various measures of blood, serum, urine, and other laboratory tests. The medical monitors or other data capture devices are linked to a database that updates and synchronizes the information presented to all of the monitors and data capture devices, and captures information from all of the monitors or data capture devices. Individual patient medical treatment plans may be remotely created, modified, or viewed in the database; and in turn are transformed by business logic rules and a communications system into prompted and recorded events that are delivered to the patient by the remote monitors. The patient monitoring data may then be combined with physician prescribing data, medication sales data, genomic, proteomic, and physiologic data for analysis and data mining purposes. In addition, the method provides for each device to record whether the patient is following the treatment plan; and to upload that recorded data to the database; which provides for monitoring the progress of treatment as the individual patient uses monitoring devices or other data capture devices. The monitored outpatient may be managed by one or more medical treatment or clinical research protocols or plans that involve; pharmaceutical drugs, physiologic data, treatment instructions, medical educational or advertising content including streaming video and other types of direct-to-consumer advertising content, medication compliance assessment, and health status or quality of life assessment. Each of these protocols or plans is first translated into a set of data points stored in a medical database and configured as a patient medical file. The data points are then translated into a sequence of time-and-event-driven, graphic and/or auditory, real-time communications presented to the patient or caregiver via remote devices that are linked to the database. The patient then interacts with remote devices that record these interactions in real-time, and communicate the recorded data to the database in real-time or via store-and-forward means. By creating medical databases containing data points that define the outpatient's medical treatment plan or protocol, which include streaming video and/or advertising content presented in a targeted fashion to specific patients or groups of patients; and then presenting this information over real-time patient monitoring devices or other data capture devices that can record the patient's responses; and in addition providing different streaming video and other advertising content presented to the prescribing physicians; the data thereby presented and derived can be analyzed and mined; and be combined with physician prescribing, drug sales, genetic, proteomic, and phenotypic data for further analysis; to determine the relationship between streaming video and/or other advertising content presented, drug sales, medication compliance, patient outcomes, and costs of treatment. The method has application in providing a much more targeted approach to direct-to-consumer and direct-to-physician advertising campaigns of pharmaceutical companies, which are designed to maximize the sales of specific medications.

Medication is widely regarded as the most cost-effective medical means of treating patients. Recently, pharmaceutical manufacturers have initiated direct-to-consumer advertising campaigns to market and sell prescription drugs to mass populations of consumers. These advertisements are delivered via television or traditional print media campaigns. The manufacturers also conduct direct-to-physician marketing and advertising campaigns. Prior to and subsequent to bringing a new medication onto the market, the pharmaceutical manufacturer must subject the drug compound to expensive, rigorous, pre-clinical and clinical tests, to determine its safety, efficacy, and costs. These tests are conducted in clinical drug trials, wherein the compound is first tested in animals, then small populations of humans, then in larger populations of humans. First the safety (incidence and severity of side effects) of the drug must be established. Then preliminary data on effectiveness (efficacy) must be established. These early safety and efficacy trials are conducted in small numbers of human study participants. Then larger trials, at times at multiple different pharmaceutical dosing levels, further define the safety and efficacy profile for the drug compound at particular doses. The average cost of bringing anew drug to market is $500,000,000, and takes an average of seven years. Each day of delay in bringing the drug onto the market costs the manufacturer $1,000,000 to $10,000,000 in lost sales, and reduces the post-market life of the drug's patent. Once the drug is on the market, post-market surveillance studies are used to better understand safety, efficacy, the prevalence of side effects, and drug interactions with other drug compounds in combination with the new drug being studied. This $500,000,000 drug development cost must be recouped as quickly as possible for a pharmaceutical company to remain competitive in the marketplace. To assist in recouping these drug development costs, direct-to-consumer and direct-to-physician advertising techniques have been enlisted, to influence physician prescribing patterns by encouraging consumers to ask their physicians to prescribe the advertised drugs, and encouraging the physicians to prescribe the drugs, respectively. In addition, new techniques of analyzing the human genome (genomics), human protein structure and function (proteomics), and the physical expression of the genes (phenotyping) are being used to help determine the specific and different responses of genetically and phenotypically different populations of patients to pharmaceutical agents, to help distinguish them in a highly competitive marketplace. It is desirable both from an ethical as well as a financial standpoint to target the treatment of a particular population of patients with the most effective drugs for that population. It is also desirable to advertise these drugs directly to that population and to their prescribing physicians, to encourage these patients to ask their doctors to prescribe them the advertised drug, and to independently encourage the doctors to prescribe the drugs. Currently these drugs are advertised by traditional television and print media campaigns to mass populations of patients, for most of whom the advertisements are irrelevant, as they don't suffer from the medical condition treated by the advertised medication. It would be useful to find a more efficient and targeted way to advertise specific drugs to specific populations of patients that are likely to use the drugs, and to their prescribing physicians, which would facilitate better positioning and branding of drugs and the pharmaceutical manufacturers. It would also be useful to monitor the reactions of these patients and physicians to the advertisements, in much the same way that Nielsen rates viewers' ratings of television programs.

Recently, various interactive technologies have been used to advertise to consumers and elicit responses from them. These same technologies are used to advertise to physicians. The technologies include personal digital assistants (PDAs), interactive television, and personal computers (PCs), through which consumers and physicians may access Web sites via the Internet. These technologies may include electronic devices that communicate with patients and be used to monitor and manage medical treatment regimens and protocols, for treating a patient's medical condition and monitoring the patient's outcomes. These devices may communicate one or more of the following: medication schedule and instruction data, medical treatment data, medical education content including visually presented content, patient query data and patient response data, and physiologic data. The devices include a controller for controlling modes of device operation, controlling access to the memory, controlling the communication of treatment data and patient query data on a display or via voice communications means, receiving and processing patient response data, tracking timing, and providing scheduled medication alarm signals. The display may present streaming video and other visual and pictorial content. The devices may include soft function keys interfaced with the controller. The soft function keys signal the controller, commanding it to execute different modes of operation of the medical monitoring device. The devices may also provide for scheduled medication alarm signals that alert the user concerning prescribed medications to be taken.

What is missing from current methods of direct-to-consumer and direct-to-physician advertising of medication is a means of targeting specific advertisements to specific patients or groups of patients, and to specific physicians and specialty physicians in real-time, based upon the relevance of these advertisements to the patients and the physicians treating them.

What is also missing is the ability to measure the effects of specific direct-to-consumer and direct-to-physician advertisements on patient medication compliance.

What is also missing is the ability to measure the effects of specific direct-to-consumer and direct-to-physician advertisements on patient health status and quality-of-life, and to combine this data with other database data such as physician prescribing data, drug sales data, genomic, proteomic, phenotypic, economic, and other healthcare related data, for further data analysis and correlation.

What is also missing is the statistical analysis or data mining of part or all of the data to correlate direct-to-consumer and direct-to-physician advertisements presented to patients and physicians, including streaming video and other audiovisual and/or visual presentations, with patient medication dosing patterns of one or more drugs ingested by the patient; and with various other measures of clinical and economic outcomes of the patients, prescribing patterns of their physicians, and drug sales to the patients.

It is therefore an object of the present invention to enter streaming video and other forms of visual and/or audiovisual advertisements into a medical database; present them to specific patients or a group of patients, and to specific physicians and specialty groups of physicians, over one or more interactive technologies; and capture the following real-time data from patients: medication compliance for one or more medications taken by the patient, patient answers to health status and quality-of-life questionnaires, patient physiologic data, and patient laboratory data; and analyze all of this data to determine correlation.

It is a further object of the present invention to provide the ability to combine this data with other database data such as patterns of physician prescribing, drug sales data, genomic, proteomic, phenotypic, economic, and other healthcare related data, for further data analysis and correlation.

It is a further object of the present invention that the statistical analysis and data mining of part or all of the data will include the method of correlating the above data following the patient developing side effects or adverse drug reactions from one or more drugs, to use the advertisements to mitigate the negative feelings of the patients and the physicians toward the medication and the pharmaceutical manufacturer.

It is a further object of the present invention that the statistical analysis and data mining of part or all of the data will include the method of correlating the above data following the patient's drug being recalled from the market.

It is a further object of the present invention that by analyzing the advertising content delivered to the patients and the physicians, along with patient monitoring, genomic, proteomic, and physiologic data, one will be able to predict the responses of sub-populations of patients and physicians to a given advertisement or combination of advertisements, based upon the specific attributes of these sub-populations. These responses will include physician prescribing patterns and drug sales results.

It is a further object of the present invention that the knowledge gained from data analysis and data mining can be used to select the most effective streaming video and other types of advertising to increase drug sales, to alter physician prescribing patterns, and to improve patient and physician satisfaction with the medication and with the pharmaceutical manufacturer. This will in turn improve the positioning and branding campaigns sponsored by the pharmaceutical manufacturers.

This method may be facilitated by translating a complex medical treatment plan of a medical outpatient into a sequential series of automated, prompt and record events presented over time; to interactive technologies that monitor a patient's responses, and present the patient's responses to their physicians over separate interactive technologies. The prompted events may include streaming video and/or audiovisual and/or visually presented advertisements. The interactive technologies are linked to a database that updates and synchronizes the information presented to them, and captures information from them. Individual patient medical treatment plans may be remotely created, modified, or viewed in the database; and in turn are transformed by business logic rules and a communications system into prompted and recorded events that are delivered to the patient by the interactive technologies. A record of the patients' responses may then be delivered to the physicians over their personal interactive technologies.

The database software may be any functional database system, for example Oracle and Microsoft. The streaming video content may be provided by any such company, such as that provided by Screaming Video Corporation. The medical data statistical analysis and data mining functions can be performed by any functional system, such as that provided by Medical Internet Solutions, Inc. The business logic rules may be any functional system, such as TEOCO Corporation's PageNgine, or DREAM (Dynamic Rapid Enterprise Application Method). The software that synchronizes the database with the patient-interactive monitoring devices may be any functional system, such as Aether Corporation's ScoutSync technology. The remote device may be any communications device such as a Personal Digital Assistant such as Palm Pilot®, cellular telephone, interactive pager, interactive television, or proprietary device such as the Med-eMonitor™.

A medical database is used to store and analyze the following data presented to and captured from patients; streaming video advertising content, visual advertising content, audiovisual advertising content, medication compliance for one or more medications taken by the patient, patient answers to health status and quality-of-life questionnaires, patient physiologic data, patient laboratory data, genomic, proteomic, phenotypic, economic, drug sales and other healthcare related data. This storage and analysis can be accomplished by utilizing a standard ODBC (Object-oriented database compliant database). The example shown below, written in Microsoft SQL Server 7, is illustrative, and should in no way be construed as limiting the invention.

The database application, in this case provided in a Microsoft SQL Server 7 database, can be converted into configuration files that are downloaded via modem, cable, infra-red, laser, computer disk, or wireless means into the patient-interactive devicesused by each individual patient. The files are translated into routines that convert the medical protocol structure into a series of prompt and record events. The prompted events may include streaming video, audiovisual, purely visual, and other types of advertisements. In this way the patients get the benefit of event-presentation on a real-time basis.

Once the caregivers and/or advertisers populate the data fields in the database by selecting a combination of data elements for the patient from an infinite number of possible data elements, they enter the data elements into fields of the database. The database converts these fields into configuration files that are downloaded into the individual patient's interactive devices, or the data may presented live and in real-time, as in the case of interactive television. In the alternative, the database may sequentially transmit the real-time prompt-and-record events directly into the patient devices. This method results in patient management protocols that can be instantaneously updated.

Advertisements, alarm-based medication events, educational content messages, alarm-based treatment instructions, questionnaires, and any other elements contained in the medical treatment plan or protocol can be delivered over time and space to the remote patient-interactive technologies in a synchronized fashion. As the patient responds to a particular device by inputting data into the device, the database synchronously updates its patient file and the treatment regimen.

The events that are then prompted and recorded can be organized and managed in an event log as illustrated below, which corresponds with the appropriate fields of the database that is prepared to accept the events from the event log. This event log listing is by no means meant to be limiting, as the event log could also include: streaming video clips; visual and/or audiovisual advertisements; specific medication; medication dosage; patient physiological measurement, for example weight, blood pressure, pulse rate, glucose level, any antigen level, pH, pO_{2,} temperature, EKG rhythm, pO2 saturation of the blood, hormone level; any psychological measurement, for example the score based upon standardized or non-standardized tests measuring anxiety, stress, anger, suicidal tendencies, schizophrenic relapse, rapid cycling bipolar relapse or confusion; medical education content related to any disease state or medical condition, for example congestive heart failure, hypertension, angina, circulatory inadequacy or other disease condition of the cardiopulmonary and circulatory systems, specific organ failure, dysfunction of an organ or system or transplanted organ such as asthma for the lungs, kidney failure for the kidney, arteriosclerosis or atherosclerosis of the heart, and transplantation of lung, kidney or heart; class of pathogen, or a specific pathogen; for example instructions or content may be based upon viral infection, or upon a specific viral disease such as HIV/AIDS, hepatitis A,B,C,D,E or G. Similarly, the instructions or content comprising the data elements may be based upon a bacterial disease agent, for example tuberculosis, malaria, cholera and meningitis; a specific microbial agent such as a virus, bacteria, mycotic infection and parasitic infection; or may be based upon the type of disease or pathology involved or the physiological system effected; for example cancer, autoimmune disease, muscular-skeletal system, or pathology of the hematopoetic, circulatory, reproductive, dermatologic, digestive, endocrine or nervous systems.

Any technology that interacts with patients and physicians, capable of communicating by modem, fax, phone line or wireless means may be used to present the streaming media and other types of advertisements, and collect the patient data that is communicated to the database for storage and for display. This technology may include Personal Digital Assistants (PDAs), Personal Computers (PCs), interactive televisions, the Palm Pilot®, laptop computers, wireless devices, and “smart phones.” Among the interactive devices is the Medi-Monitor® described in PCT patent application WO98/38909 herein incorporated by reference. The database containing the advertising content, patient information, physician information, and protocols may also be the site of the medical and economic data, prescribing patterns data and the sales data, used by the methods of the present invention.

For example, using the above method, a streaming video presentation about how the heart functions before and after treatment with Digoxin (a medication that increases the strength and efficiency of heart muscle) could be presented to heart failure patients of a specific age, gender, and race. A different presentation would be given to the physicians, who may be general practitioners, internists, or cardiologists. The purpose of the presentations to the patients would be to improve the medication compliance of this subgroup of patients by showing them how the Digoxin improves the heart's function, reduces symptoms, and increases quality-of-life. The purpose of the presentations to the doctors would be to encourage them to prescribe Digoxin over some other competing brand. This method would likely increase sales of Digoxin, as the patients will consume more Digoxin over a given time period. Additional video content or advertisements may be presented to the patients related to other drugs taken by the patients. Heart failure patients may typically require the following medications and medical content in their medical treatment plan, with information about each to be communicated at a particular time; a beta-blocker, ACE inhibitor, diuretic, and Digoxin medication; dosing instructions for each medication; a description of each medication and rationale for taking the medication; what the medication looks like along with its color; daily measures of weight, blood pressure, pulse, and blood oxygen levels; a salt restricted, low fat, low calorie diet; an exercise program tailored to their specific level of heart failure; questionnaires that assess their shortness of breath, chest pain, energy level, and swelling of the legs; information updates about new drugs used to treat the condition; information about when to call the doctor with specific complaints or side effects or adverse drug reactions; and other information. The current method will enable the direct-to-consumer streaming video or other advertising content to include these subjects for discussion, and present the effects of the drug as positively impacting the debilitating symptoms of congestive heart failure. The method would also ensure that the correct advertising content is selected for a specific sub-population of patients (in this case heart failure patients), such that the optimum medication compliance, patient outcomes, perceptions toward a pharmaceutical manufacturer and drug sales will result. The method may also increase the prescription of Digoxin by the physicians treating these patients, as patients will be more likely to ask their physicians to prescribe Digoxin for the treatment of their congestive heart failure after having viewed the advertising. The physicians in turn would have received complementary advertisements to encourage them to prescribe the medication.

H. Enhanced Validity in Pharmaceuticals and Drug Package Inserts

In an embodiment of the present invention, system 100 is configurable to enhance scientific validity in pharmaceutical advertisement claims and drug package inserts preferably, but not necessarily, as described in commonly assigned U.S. patent Ser. No. 60/231,828, filed Sep. 12, 2000, (herein referred to as the '828 application). The disclosure of the '828s application is incorporated herein by reference as though set forth in its entirety.

The present invention describes methods and apparatus useful in enhancing pharmaceutical advertisements, drug positioning and branding, and drug package inserts based upon statistical analysis and data mining of data contained in a medical database. The medical database may contain the following real-time data captured from patients: medication compliance for one or more medications taken by the patient, patient answers to health status and quality-of-life questionnaires, side effects ratings of the medications, adverse drug events, drug interactions, patient physiologic data, and patient laboratory data. This data may also be combined with other database data such as genomic, proteomic, phenotypic, economic, drug interaction, and other healthcare related data, for further data analysis. The statistical analysis and data mining includes the method of correlating the following patient-related data; medication dosing patterns of one or more ingested drugs, and various other measures of clinical and economic outcomes, and then using this information to more accurately differentiate the advantages of a particular pharmaceutical agent over its competitors, and more accurately position and brand the pharmaceutical agent. In addition, the method provides more accurate information with respect to claims made about a particular drug, and in particular the ability to correlate the incidence of side effects, adverse drug events and drug interactions with medication compliance rates of patients who experience these unwanted events. The method may based in translating a complex medical treatment plan of a medical outpatient into a sequential series of automated, prompt and record events presented over time; to medical monitors or other data capture devices used by patients. The patients are presented the events via their personal computers (PCs), personal digital assistants (PDAs), interactive televisions, Palm Pilots®, or other medical monitoring technologies. The patient's medical monitors capture data on medication compliance, side effects, adverse drug events, drug interactions, health status, quality-of-life, physiologic status (e.g. blood pressure, EKG, pO2, pulse rate, weight, pulmonary function, etc.), and various measures of blood, serum, urine, and other laboratory tests. The medical monitors or other data capture devices are linked to a database that updates and synchronizes the information presented to all of the monitors and data capture devices, and captures information from all of the monitors or data capture devices. Individual patient medical treatment plans may be remotely created, modified, or viewed in the database; and in turn are transformed by business logic rules and a communications system into prompted and recorded events that are delivered to the patient by the remote monitors. The patient monitoring data may then be combined with physician prescribing data, medication sales data, genomic, proteomic, and physiologic data for analysis and data mining purposes. In addition, the method provides for each device to record whether the patient is following the treatment plan; and to upload that recorded data to the database; which provides for monitoring the progress of treatment as the individual patient uses monitoring devices or other data capture devices. The monitored outpatient may be managed by one or more medical treatment plans or clinical research protocols that involve; pharmaceutical drugs, side effects ratings scales, adverse drug event detection, drug interaction detection, physiologic data, treatment instructions, medical educational content medication compliance assessment, and health status or quality of life assessment. Each of these protocols or plans is first translated into a set of data points stored in a medical database and configured as a patient medical file. The data points are then translated into a sequence of time-and-event-driven, graphic and/or auditory, real-time communications presented to the patient or caregiver of the patient via remote devices that are linked to the database. The patient or caregiver then interact with remote devices that record these interactions in real-time, and communicate the recorded data to the database in real-time or via store-and-forward means. By creating medical databases containing data points that define the outpatient's medical treatment plan or protocol, and then presenting this information over real-time patient monitoring devices or other data capture devices that can record the patient's responses; the data thereby presented and derived can be analyzed and mined; and be combined with physician prescribing, drug sales, genetic, proteomic, and phenotypic data for further analysis; to determine the correlation between drug sales, medication compliance, patient outcomes, and costs of treatment. The method has application in providing more scientifically valid information about medication effects for use in advertising campaigns of pharmaceutical companies, which are designed to maximize the sales of specific medications, in an increasingly competitive marketplace. In addition, the method has application in providing more accurate information for drug package inserts mandated by the Food and Drug Administration for all prescription medications.

Medication is widely regarded as the most cost-effective medical means of treating patients. Pharmaceutical manufacturers have initiated advertising campaigns to influence the prescribing patterns of physicians, and market and sell prescription drugs to mass populations of consumers. These advertisements are delivered to physicians via detailed salespeople and trade journals; and are delivered to patients via television or traditional print media campaigns. In addition, the Food and Drug Administration (FDA) mandates that each pharmaceutical manufacturer provide a package insert for all prescription and non-prescription drugs, that describes the basic characteristics of the drug, its indications for use, the percentage of patients suffering from side effects and adverse drug reactions caused by the drug, drug interactions warnings, dosing information, risk of carcinogenesis, and other information. Prior to and subsequent to bringing a new medication onto the market, the pharmaceutical manufacturer must subject the drug compound to expensive, rigorous, pre-clinical and clinical tests, to determine its safety, efficacy, and costs. These trials also may compare the new drug to existing competitors' drugs to differentiate the new drug based upon certain clinical advantages over the competitors. The average cost of bringing a new drug to market is $500,000,000, and takes an average of seven years. Each day of delay in bringing the drug onto the market costs the manufacturer $1,000,000 to $10,000,000 in lost sales, and reduces the post-market life of the drug's patent. Once the drug is on the market, post-market surveillance studies are used to better understand safety, efficacy, the prevalence of side effects, and drug interactions with other drug compounds in combination with the new drug being studied. Post market studies also compare the drug to competitors' drugs, in an effort to gain more market share. This $500,000,000 drug development cost must be recouped as quickly as possible for a pharmaceutical company to remain competitive in the marketplace. To accelerate the recouping the cost of drug development, direct-to-physician and direct-to-consumer advertising techniques have been enlisted, to influence physician prescribing patterns, and to encourage consumers to ask their physicians to prescribe the advertised drugs. In addition, new techniques of analyzing the human genome (genomics), human protein structure and function (proteomics), and the physical expression of the genes (phenotyping) are being used to help determine the specific and different responses of genetically and phenotypically different populations of patients to pharmaceutical agents, to help distinguish them in a highly competitive marketplace. It is desirable both from an ethical, clinical, and financial standpoint to target the treatment of a particular population of patients with the most effective drugs for that population. It is also desirable to emphasize a drug's competitive advantage over other drugs in terms of increased effectiveness, better quality-of-life, and fewer side effects and adverse drug reactions. It is also desirable to advertise these drugs' competitive advantages directly to patients and to their prescribing physicians. Currently these drugs are distinguished from one another and advertised using information derived from traditional clinical research techniques, that lack to ability to provide information on the real-time correlation between medication compliance, patient health outcomes, and patient quality-of-life. (Please reference attached article “Electronic Monitoring of Medication Adherence,” from the journal, “Epimonitor—The Epidemiology Monitor, Volume 19, Number 10, November 1998.”) For example, in traditional drug trials, the degree to which patients in those trials are complying with or not complying with their medication regimens is poorly measured, if at all. Even if medication compliance is measured, it is not correlated in real-time with measures of side effects, drug interactions, adverse drug events, quality-of-life, health outcomes, and other measures of patient satisfaction and well being. A drug that appears to be producing side effects may in fact be taken too frequently by the patients in the trial, thus the drug is unfairly blamed for the side effects, when it is the patients' inadequate dosing that is causing the problem. Conversely, a drug that appears to be relatively ineffective in treating the patients' conditions may in fact be taken too infrequently by the patients, and if it were properly administered, may be highly effective. An example of this is with the category of drugs called protease inhibitors, which are used to treat HIV/AIDS patients. The protease inhibitor is a potent antiviral agent that prevents the HIV virus from duplicating itself. It is well known that these drugs are also capable of inducing mutated and resistant HIV viruses that do not respond to any drug therapy, if the patient does not reliably take these drugs. It is unknown under present methods how much and what types of noncompliance cause the development of resistant and mutant HIV viruses (e.g. is it a 2 day drug holiday, 4 day holiday, missing each evening's dose for 6 days in a row, etc.?). At the same time these patients are on multiple other antiviral and antibiotic drugs. What is the influence of these drug combinations on the HW virus mutation rate if they are taken properly, or improperly? These drug “cocktails” are also known to produce side effects and quality-of-life challenges, which could encourage medication non-compliance in the patients. If non-compliance develops with one or more of these drugs, or the drugs in combination are not taken appropriately, the drug is blamed when in fact the patient is responsible for the perceived lack of drug effectiveness, resistant/mutated HIV virus, or poorer quality-of-life. As well, a drug may be unfairly blamed for side effects caused by excess dosing of another drug, or caused by drug interactions. Finally, the quality-of-life produced by a drug is a critical issue to measure, and would be a distinguishing characteristic among drugs with competitors on the market. Current methods do not correlate medication compliance of the patient with quality-of-life measures in real-time. The problem is then compounded when the information gathered in traditional clinical trials is then used in advertising, detailed sales to doctors, and patient package inserts, replete with all the potential inaccuracies and problems noted above (c.f. Epimonitor article). It would be useful to find a more accurate method to capture and correlate data for a particular drug, upon which advertising claims and patient package inserts could be based. It would also be useful to find a more accurate method to capture and correlate data for comparing a particular drug to its competitors, including which drug produces a better quality-of-life, and fewer side effects and adverse drug interactions; all of which could be incorporated into advertising, branding and positioning the drug in a more favorable way. This method should enhance the accuracy and scientific basis of pharmaceutical advertising claims and drug package inserts, and when adopted by a pharmaceutical company, would positively distinguish that company from its competitors. In other words, part of the branding and positioning strategy of the pharmaceutical manufacturer itself would involve them claiming that they use a more scientific method of studying drug effects, and claiming the superiority of their drug compounds.

Recently, various interactive technologies have been used to monitor patients. The technologies include personal digital assistants (PDAs), interactive television, and personal computers (PCs), through which consumers connect to the Internet. These technologies may include electronic devices that communicate with patients and be used to monitor and manage medical treatment regimens and protocols, for treating a patient's medical condition and monitoring the patient's outcomes. These devices may communicate one or more of the following: medication schedule and instruction data, medical treatment data, medical education content including visually presented content, patient query data and patient response data, and physiologic data. The devices include a controller for controlling modes of device operation, controlling access to the memory, controlling the communication of treatment data and patient query data on a display or via voice communications means, receiving and processing patient response data, tracking timing, and providing scheduled medication alarm signals. The display may present streaming video and other visual and pictorial content. The devices may include soft function keys interfaced with the controller. The soft function keys signal the controller, commanding it to execute different modes of operation of the medical monitoring device. The devices may also provide for scheduled medication alarm signals that alert the user concerning prescribed medications to be taken.

What is missing from current methods of direct-to-consumer and direct-to-physician advertising of medication is a means of differentiating a particular medication from its competitors based upon the use of real-time information about the medication and its effects that is incorporated into the advertisements.

What is also missing is the ability to use real-time information about the medication and its effects that is incorporated into drug package inserts.

What is also missing is the use of real-time information about the medication and its effects on quality-of-life, and a comparison of quality-of-life effects of one particular medication with another medication or class of competitive medications, that is incorporated into the advertisements.

What is also missing is the ability to measure the real-time effects of medication compliance ratios on patient health status and quality-of-life, and to combine this data with other database data such as physician prescribing data, drug sales data, genomic, proteomic, phenotypic, economic, and other healthcare related data, for further data analysis and correlation, and use in advertising, positioning, and branding a medication.

What is also missing is the capability of a pharmaceutical company to claim in its positioning and branding of the company that it is using research tools that combine real-time assessment of medication compliance with real-time assessment of other measures of patient health status, to provide a more scientific basis for its drug advertising claims than its competitors.

It is therefore an object of the present invention to capture the following real-time data from patients: medication compliance for one or more medications taken by the patient, patient answers to health status and quality-of-life questionnaires, patient physiologic data, and patient laboratory data; and analyze all of this data to use it in advertising and drug package inserts, to differentiate a particular drug from competitive drugs.

It is a further object of the present invention to provide the ability to combine this data with other database data such as patterns of physician prescribing, drug sales data, genomic, proteomic, phenotypic, economic, and other healthcare related data, for further data analysis and correlation; to be used to better position and brand a drug against its competitors.

It is a further object of the present invention that the statistical analysis and data mining of part or all of the data will include the method of correlating the above data following the patient developing side effects, drug interactions, or adverse drug reactions from one or more drugs, to use this information in advertisements to better differentiate a drug from its competitors, and more accurately assess the drug's contributions toward negative effects experienced by the patient. This will serve to increase the competitive advantage of the drug, and prevent it from being unfairly blamed for problems caused by other factors.

It is a further object of the present invention that the statistical analysis and data mining of part or all of the data will include the method of correlating the above data following the patient's drug being recalled from the market, to determine whether it is the drug that caused the adverse drug events when taken at the prescribed dosage, or some other factor. The present method will enable the researcher to distinguish whether the adverse drug event is due to; the patient taking too much drug, drug interactions, genomic, proteomic, phenotypic, or other factors unrelated to the drug per se.

It is a further object of the present invention that by analyzing the patient monitoring, genomic, proteomic, and physiologic data, one will be able to predict the responses of sub-populations of patients and physicians to a given drug or combination of drugs, and be able to use this information in advertisements about the drug as well as drug package inserts to better position and brand the drug for these sub-population of patients.

It is a further object of the present invention that the above methods and information gathered can be used by the pharmaceutical manufacturer to better position and brand itself versus its competitors, by asserting that it uses a more scientific method of testing the safety and efficacy of its pharmaceutical compounds.

This method may be facilitated by translating a complex medical treatment plan of a medical outpatient into a sequential series of automated, prompt and record events presented over time; to interactive technologies that monitor a patient's responses, and present the patient's responses to their physicians over separate interactive technologies. The prompted events may include streaming video and/or audiovisual and/or visually presented content. The interactive technologies are linked to a database that updates and synchronizes the information presented to them, and captures information from them. Individual patient medical treatment plans may be remotely created, modified, or viewed in the database; and in turn are transformed by business logic rules and a communications system into prompted and recorded events that are delivered to the patient by the interactive technologies. Data analytic and data mining software is then used to analyze the data, which is provided to the sales and marketing department of the pharmaceutical manufacturer, which in turn incorporates the information into consumer and physician advertisements, drug positioning and branding statements, drug package inserts, and positioning and branding of the pharmaceutical manufacturer itself. All of these advertisements may be presented through any currently available or future media including Internet, television, print, and radio adverttisements.

The database software may be any functional database system, for example Oracle and Microsoft. The streaming video content may be provided by any such company, such as that provided by Screaming Video Corporation. The medical data statistical analysis and data mining functions can be performed by any functional system, such as that provided by Medical Internet Solutions, Inc. The business logic rules may be any functional system, such as TEOCO Corporation's PageNgine, or DREAM (Dynamic Rapid Enterprise Application Method). The software that synchronizes the database with the patient-interactive monitoring devices may be any functional system, such as Aether Corporation's ScoutSync technology. The remote device may be any communications device such as a Personal Digital Assistant such as Palm Pilot®, cellular telephone, interactive pager, interactive television, or proprietary device such as the Med-eMonitor™.

A medical database is used to store and analyze the following data presented to and captured from patients; streaming video content, visual content, audiovisual content, medication compliance for one or more medications taken by the patient, patient answers to health status and quality-of-life questionnaires, side effects data, adverse drug events data, drug interactions data, patient physiologic data, patient laboratory data, genomic, proteomic, phenotypic, economic, drug sales and other healthcare related data This data includes real-time data captured from the patient, which may be extracted and correlated with non-real-time data, to create more rigorous conclusions about the effects of a medication. These more rigorous and scientific conclusions can then be incorporated into drug advertising and patient package inserts. This storage and analysis can be accomplished by utilizing a standard ODBC (Object-oriented database compliant database). The example shown below, written in Microsoft SQL Server 7, is illustrative, and should in no way be construed as limiting the invention.

The database application, in this case provided in a Microsoft SQL Server 7 database, can be converted into configuration files that are downloaded via modem, cable, infra-red, laser, computer disk, or wireless means into the patient-interactive devicesused by each individual patient. The files are translated into routines that convert the medical protocol structure into a series of prompt and record events. The prompted events may include streaming video, audiovisual, purely visual, and other types of advertisements. In this way the patients get the benefit of event-presentation on a real-time basis.

Once the caregivers and/or advertisers populate the data fields in the database by selecting a combination of data elements for the patient from an infinite number of possible data elements, they enter the data elements into fields of the database. The database converts these fields into configuration files that are downloaded into the individual patient's interactive devices, or the data may presented live and in real-time, as in the case of interactive television. In the alternative, the database may sequentially transmit the real-time prompt-and-record events directly into the patient devices. This method results in patient management protocols that can be instantaneously updated.

Advertisements, alarm-based medication events, educational content messages, alarm-based treatment instructions, questionnaires, and any other elements contained in the medical treatment plan or protocol can be delivered over time and space to the remote patient-interactive technologies in a synchronized fashion. As the patient responds to a particular device by inputting data into the device, the database synchronously updates its patient file and the treatment regimen.

The events that are then prompted and recorded can be organized and managed in an event log as illustrated below, which corresponds with the appropriate fields of the database that is prepared to accept the events from the event log. This event log listing is by no means meant to be limiting, as the event log could also include: streaming video clips; visual and/or audiovisual advertisements; specific medication; medication dosage; patient physiological measurement, for example weight, blood pressure, pulse rate, glucose level, any antigen level, pH, pO₂, temperature, EKG rhythm, pO2 saturation of the blood, hormone level; any psychological measurement, for example the score based upon standardized or non-standardized tests measuring anxiety, stress, anger, suicidal tendencies, schizophrenic relapse, rapid cycling bipolar relapse or confusion; medical education content related to any disease state or medical condition, for example congestive heart failure, hypertension, angina, circulatory inadequacy or other disease condition of the cardiopulmonary and circulatory systems, specific organ failure, dysfunction of an organ or system or transplanted organ such as asthma for the lungs, kidney failure for the kidney, arteriosclerosis or atherosclerosis of the heart, and transplantation of lung, kidney or heart; class of pathogen, or a specific pathogen; for example instructions or content may be based upon viral infection, or upon a specific viral disease such as HIV/AIDS, hepatitis A,B,C,D,E or G. Similarly, the instructions or content comprising the data elements may be based upon a bacterial disease agent, for example tuberculosis, malaria, cholera and meningitis; a specific microbial agent such as a virus, bacteria, mycotic infection and parasitic infection; or may be based upon the type of disease or pathology involved or the physiological system effected; for example cancer, autoimmune disease, muscular-skeletal system, or pathology of the hematopoetic, circulatory, reproductive, dermatologic, digestive, endocrine or nervous systems.

Any technology that interacts with patients and physicians, capable of communicating by modem, fax, phone line or wireless means may be used to present the streaming media and other types of advertisements that are part of the positioning and branding of the drugs, and collect the patient data that is communicated to the database for storage and for display. This technology may include Personal Digital Assistants (PDAs), Personal Computers (PCs), interactive televisions, the Palm Pilot®, laptop computers, wireless devices, and “smart phones.” Among the interactive devices is the Medi-Monitor® described in PCT patent application WO98/38909 herein incorporated by reference. The database containing the advertising content, patient information, physician information, and protocols may also be the site of the medical and economic data, side effects data, adverse drug events data, drug interaction data, prescribing patterns data and the sales data, used by the methods of the present invention.

For example, using the above method, the analyzed and mined data may be incorporated into a streaming video presentation about how the heart functions before and after treatment with Digoxin (a medication that increases the strength and efficiency of heart muscle), to be presented to heart failure patients of a specific age, gender, and race. A different presentation would be given to the physicians, who may be general practitioners, internists, or cardiologists. The purpose of the presentations to the patients and physicians would be to improve the positioning and branding of the Digoxin, to improve its sales and improve the medication compliance of this subgroup of patients by showing them how the Digoxin improves the heart's function, reduces symptoms, and increases quality-of-life. The purpose of the presentations to the doctors would be to encourage them to prescribe Digoxin over some other competing brand through demonstrating a superior method of clinical drug trials conduct, which in turn better positions and brands the pharmaceutical company in the mind of the physician. This method would likely increase sales of Digoxin, as the patients will consume more Digoxin over a given time period. Finally, the use of this method of research by the pharmaceutical company in creating more accurate drug package inserts would make the FDA look more favorably upon the company's business practices, which is very important in this highly regulated industry.

I. Mass Customizing Information Device Functions and Features

In an embodiment of the present invention, system 100 is configurable for mass customization of information devices used by subgroups preferably, but not necessarily, as described in commonly assigned U.S. patent Ser. No. 60/260,231, filed Jan. 8, 2001, (herein referred to as the '231 application). The disclosure of the '231 application is incorporated herein by reference as though set forth in its entirety.

The present invention describes methods and apparatus useful in remotely modifying the functional attributes of information devices used by subgroups that are defined by specific database characteristics. It is well known that different subgroups have different device use preferences, desires for information content and data analysis, user abilities, degrees of technologic and intellectual sophistication, cognitive abilities or impairments, and price-point sensitivities. As well, various regulatory agencies may regulate the use of these devices based upon their being dedicated purpose vs. general purpose devices (e.g. the FDA prefers dedicated information devices in clinical trials that serve one and only one function, that of data capture for the trial). The functional attributes of the remote information devices may assist the subgroups in efficiently managing various activities of daily life, which may include correctly adhering to medical treatment plans or protocols; improving their personal health and wellness promotion activities; better managing their workplace tasks for specific vertical industries; and other activities that are process-driven and information-based. The invention provides for a series of remote devices linked to one or more servers via the Internet, an intranet, or other means of connectivity between client and server. The connectivity may be established via hard wire modem, wireless, light beam, fiber-optic cable, or other means. The invention provides for remotely creating a continuum of device features and functionality that is made available to the user of the device on a subgroup-specific basis; ranging from thin-client devices that host no software applications, and run entirely from server-based software applications; to PC-like devices that host many software applications that run independently of the server, whether the devices are connected to the server or not. Software applications may be remotely downloaded to the client device from the server based upon database characteristics of the device users (e.g. via downloadable Java applets); or the applications may be remotely accessed and used via an ASP (Application Services Provider) service model. In the latter case, the software applications are hosted on the remote server, and are installed, maintained and enhanced by a third party. The economics of providing the continuum of device functions and features for specific subgroups may be supported via software license fees in the case of software applications downloaded to the device; or via subscription or per-use fees in the case of thin-client connectivity supported by ASP software access. The method therefore supports different pricing models for a large variety of sponsored programs that may enroll and financially support the different subgroups' use of the information devices. The method has application in many fields of life, for example the medical management of subgroups of outpatients. These outpatients may be managed by one or more medical treatment or clinical research protocols or regimens involving pharmaceutical drugs, physiological data, educational content, and health status assessment or quality of life questionnaires. In addition, the present method provides for a variety of different medication containers that may be used by the user, that communicate with the information device. These containers differ in size and functionality, to accommodate the differing preferences and medication regimens of the users. Each container records and date-and-time-stamps the medication compliance of the user, and communicates this data with the information device. The container may also provide other information to the user, such as when to take medication, which medication to take, how many to take, and other special instructions. This method thereby further expands the range of options available to the user, and/or to those who manage the user's health. This method of mass customization of medical device features and functions used by these outpatients, said features and functions selected by subgroup characteristics present in a medical database, is useful in efficiently monitoring and managing one or more subgroups of medical outpatients. Through simultaneously providing specific device functionality to selected patient subgroups present in the database, the correct functionality can be assigned to the correct subgroup. This method would result in more targeted and personalized support of the patient, more flexibility in delivering information to the patient and caregiver, more flexibility in the pricing and costs of such services, and more effective healthcare delivery.

Various electronic devices may be used to provide information to users of the devices. These devices range from “thin client” or “dumb terminal”devices that primarily serve to connect the user to a server that hosts a variety of software applications; to “smart devices” or “Personal Digital Assistants” or “Personal Computers” that host a variety of software applications themselves, and may also connect to a server that hosts even more complex, elaborate and expensive software applications that may be accessed and used. The devices include a controller for controlling modes of device operation, controlling access to the memory, controlling display of data and information on a display, receiving and processing data contained in databases, and downloading software applications from servers, or connecting to servers to use software applications residing on the servers via an ASP (Application Services Provider) model. It is well known that different users have widely different needs and preferences regarding the functions and features of these devices, both in terms of their hardware design, and the software applications they host. It is also well known that different users have different needs for specific information that pertain to their particular life circumstances, and that their appetite for, and ability to understand, the myriad array of information specifically relevant to them will vary widely based upon demographic characteristics, education level, socioeconomic status, cognitive abilities, personal preferences, etc. It is also well known that information, specifically relevant and properly applied, can enhance the life experience and activities of daily living of these individuals, through enhanced work performance, enhanced financial well-being, improved healthcare and wellness activities, improved business processes, etc.

While using various information devices to improve the activities of daily life, various subgroups of users often face the need to manage a complicated series of tasks, as defined by protocols, business processes, systems, or regulations. These protocols or processes require the users to carry out a detailed series of behaviors in a sequential fashion throughout the day, related to accomplishing specific tasks or goals. Some of these tasks and goals are population-specific, and apply to entire populations of users with specific life needs and goals, either at work or in their personal lives.

At present, via hardware and software sales through retail stores, catalogues, downloads via the Internet, and via remote server access by the devices, users can avail themselves of a bewildering array of software applications to enhance the above-noted activities of daily living. Unfortunately, the choices available are immense in number, not targeted toward specific subgroups of users based upon their specific needs and characteristics, and therefore not the most efficient, fastest, and most cost-effective way to serve the information needs of the users.

The present invention solves this problem by providing for mass customization of information device functions and features, and software applications for use by those devices and users, based upon specific user subgroup characteristics contained in a database; through downloading specific software to the devices of specific user subgroups, and/or providing specific user subgroups access to specific software applications hosted in a remote server.

The software applications are selected to serve specific subgroups based upon data that demonstrates the greatest utility for the least cost for the user subgroup under consideration.

Such mass customization of device functions and features would enable the device users to get the right information at the right time via the device, to sequentially follow and manage a complex protocol or process. The software applications would enable the user to optimally understand the protocol or process, and would shape the behavior of the user to produce the best outcomes. The mass customization would first be based upon the assignment of specific software applications to specific user subgroups, based upon expert knowledge of the software applications best suited to a particular subgroup. Once the assignment was made, upon downloading to the devices of specific subgroups the specific software applications that facilitate the management of their particular challenges; and/or providing them access via an ASP model to specific software applications hosted in a remote server; the user could begin to use the software to implement the process. A continuum of software application access and information services could be provided to the user devices, ranging from the thin client to the “rich” client devices, with a variety of price points established depending upon the sophistication of the software applications provided. The selection of which applications would be provided to specific subgroups would be made based upon the knowledge of experts familiar with the subgroups and their needed protocols and processes, subgroup user satisfaction data, subgroup performance evaluations, subgroup quality-of-life measures, subgroup economic constraints, and other metrics.

It is therefore the object of the present invention to enable experts, subgroup users and others to select specific software applications to be made available for downloading to the information devices used by specific user subgroups, based upon data contained in a database that identifies and organizes these subgroups into groups.

It is the further object of the present invention to enable experts, subgroup users and others to select additional specific software applications to be made available for downloading to the information devices of specific individuals, to supplement and complement the subgroup-assigned software.

It is therefore the object of the present invention to enable experts, subgroup users and others to select specific software applications to be made available via an ASP model server connection to the information devices used by specific user subgroups, based upon data contained in a database that identifies and organizes these subgroups into groups.

It is the further object of the present invention to enable experts, subgroup users and others to select additional specific software applications to be made available via an ASP model server connection to the information devices of specific individuals, to supplement and complement the subgroup-assigned software.

It is a further object of the present invention that the software applications used by the information devices, to assist specific user subgroups, be designed to optimize the management of complex processes and behaviors of the user subgroups, based upon knowledge of which software applications will prove most useful to the specific subgroups.

It is the further object of this invention that the subgroup-specific software applications be accessed via the use of search engines that search other databases for applications that are specific to the population or subgroup. This search and retrieve function can be applied to educational content management, self-management instructions, decision support software, or any other software that will improve the life of the users belonging to the subgroup.

It is the further object of this invention that the software applications be remotely accessed for download or use, by accessing the databases via direct dial-in or Internet access; and that the software applications, once selected for the specific subgroups, be remotely transmitted to, or accessible via ASP server communication to, a series of possible information devices, including personal digital assistants (PDAs) such as the Palm Pilot®, cellular telephones, pagers, dumb terminals, Internet appliance devices, interactive televisions, and custom-manufactured medical monitoring devices such as the Medi-Monitor® System. The remote access of the software application could be accomplished by accessing the database via any of these devices.

It is the further object of the present invention to provide for a variety of different medication containers that may be used by the user, that communicate with the information device used by the user. These medication containers may differ in size and functionality, to accommodate the differing preferences and medication regimens of the users. Each container records and date-and-time-stamps the medication compliance of the user, and communicates this data with the information device. The container may also provide other information to the user, such as when to take medication, which medication to take, how many to take, and other special instructions. For example, the containers may respectively hold one day's, one week's or one month's supply of medications. The containers may have a clock and related circuitry, to record whether and when the patient took his or her medication. The containers may have a display that gives information about when to take medication, which medication to take, how many to take, and other instructions and educational content. The display may also provide for the ability of the user to answer queries. In each instance, the containers may have two way communication with the information device via a serial port connection, infra-red or other light beam, wireless means (e.g. Blue Tooth Standard) or other means of connectivity or communication.

Once the software application(s) is transmitted to these information devices, the device operating system can be used to operate the software applications, which can be used to provide educational content, data analytic algorithms, decision support assistance, and a series of messages and queries to assist the subgroup user in following the protocol, by prompting and monitoring a series of events throughout the day. In the alternative, a wireless signal can carry the application software for direct insertion into the wireless device. The subgroup user then interacts with the information devices, which in turn communicate collected information back to the database for report-generation to the experts evaluating the efficacy to the subgroup-specific software packages or subgroup-specific software “suites.” New mass customization of the subgroup-specific software suites can then take place based upon the collected data from the user, and other measures of their effectiveness in following a protocol or process. In this fashion a complex protocol that requires a sequential set of actions and monitoring activities can be managed.

The sub-populations or groups may be any group that share one or more common characteristic. Among the groups to consider are defined by age; gender; occupation; education level; location; protocol performance level; technology sophistication; disease state; medical history event. The group may be based upon the same or similar disease state or medical condition, for example congestive heart failure, hypertension, angina, circulatory inadequacy or other disease condition of the cardiopulmonary and circulatory systems. The group may be based upon specific organ failure or dysfunction or upon a transplanted organ, for example asthma for the lungs, kidney failure for the kidney, arteriosclerosis or atherosclerosis of the heart, and transplantation of lung, kidney or heart. The group may be passed upon class of pathogen, or a specific pathogen. For example the group may be based upon viral infection, or upon a specific viral disease such as HIV/AIDS, hepatitis A,B,C,D,E or G. Similarly, the group may be based upon a bacterial disease agent, for example tuberculosis, malaria, cholera and meningitis. Groups may be defined by a specific microbial agent such as a virus, bacteria, mycotic infection and parasitic infection. The group may be based upon the type of pathology involved or the physiological system effected; for example cancer, autoimmune disease, muscular-skeletal system, or pathology of the hemopoetic, circulatory, reproductive, digestive, endocrine or nervous systems.

The groups may be based upon two or more parameters, for example airline pilot and age of pilot; software engineer and networking environment; teacher and grade level being taught; senior citizen and hardware environment used; HIV/AIDS and immune function, liver dysfunction and hepatitis, or bone degeneration and arthritis. The groups may be based upon any parameter available to the database, either directly in the database or accessible through linkage to another database or databases, and associated with a specific subgroup characteristic.

In each situation, the subgroup members' software applications will be selected and changed based upon the users' inclusion in such a group. The change submitted to the group database files will modify each user's software application selection in the same manner. Each individual user may be a member of many different sub-groups, each of which may have a change entered into the database. The changed software application suite is then uploaded to the monitoring device. Such uploading may be immediate, or it may be at the next scheduled uploading event.

For example, a subgroup of elderly heart failure patients with mild cognitive impairment would be presented an information device and software access that simply instructed them on what to do with their medication regimen, and asked them simple questions. It may also prompt them to take their weight and/or blood pressure and enter it into the information device. In this case the device would be a thin client dedicated to perform only these two functions, and would be relatively low priced. An example at the other extreme could be a highly intelligent and sophisticated subgroup of heart failure patients, for example college graduates, who are overweight, with high cholesterol levels and high blood pressure who had just suffered their first heart attack and are in mild heart failure. This subgroup would be presented a client device with a suite of very sophisticated software applications, either downloaded to the device, or accessed via an ASP server model. The information device would give instructions and queries, sophisticated medical content about heart disease management, nutritional counseling, individualized record of nutrition correlated with impact on clinical outcome, access to detailed database information specific to this population, statistical analyses of the relation between caloric and fat intake and risk factors for future heart attacks, body fat index calculations, risk factor reduction calculations, drug comparison data, algorithmic triggers of warning messages to the user based upon analysis of inputted data, and other sophisticated functionality. Some of these functions could be hosted on the device, and others could be accessed via wireless or other connectivity to one or more databases. In this case the greatly expanded device functionality would command a premium price.

Data analytic techniques would be applied to determine which combination of device functions produced the best outcomes for specific subgroups. Then, based upon this cost-effectiveness outcome data, the device-specific functionality and pricing would be applied to all members of the selected medical subgroup.

Comparable ranges of device functionality from low-level functions to extreme sophistication could be provided to users of other applications, for example airport employees ranging from gate personnel to airplane maintenance personnel to pilots. This method facilitates remote management or change of the many user device functions, by allowing selected subgroups of users to all receive the same change in device functionality, but requiring only one change process by the database administrator. By using the mass customization method whereby subgroup selected device functionality is determined, the optimum device functions and pricing models may be determined for each subgroup.

The database software may be any functional system, for example Oracle 81 and Microsoft SQL Server 7.

The mass customization of the software applications, which are then made available to remote devices via download or ASP based server communications, can be accomplished utilizing a standard ODBC compliant database, modem-based communications, and a variety of information devices noted above. The example shown below, written in Microsoft SQL Server 7, is illustrative, and should in no way be construed as limiting the invention.

As illustrated in the above system diagram and database block diagram, the goal of mass customization of mass customized access to subgroup-specific software application suites may be realized as follows. The SQL Server database can contain the software application suites for one or more user subgroups. The access control component can control which database users can access the database to assign the users to the software applications, and can also control which device user subgroups gain access via the ASP model to which suites of software applications. The PageNgine software enables different types of information devices with different characteristics to manipulate data and downloads via the Internet and a browser interface. The web server and dialup server facilitate communication between the information device and the database containing the suites of software applications. The addition of pull-down menus to the fields of the database, that populate the fields with subgroup-specific standardized software suites, further simplifies the mass customization method.

The database application of the mass customization system, in this case provided in a Microsoft SQL Server 7 database, can be used to convert suites of software applications into Java applets that are downloaded via modem, cable, or wireless means into the user information devices, which then host the software applications. The software applications may then be used to translate complex behavioral and cognitive processes into routines that convert the protocol structure into a series of prompted and recorded events.

Once the experts populate the fields in the database with specific software applications, the database converts these fields into packets (e.g. Java applets) that are downloaded into the devices so that the software application enhanced management protocols can be instantaneously updated.

In addition the user's device may receive software applications that are retrieved from other databases; via the use of search engines that search other databases for content that is specific to a population or sub-population, or to an individual. The search engine can carry our its search based upon a single characteristic of a population, two or more simultaneous characteristics of a sub-population, or a combination of specific characteristics that are particular to an individual subgroup user.

Any information device capable of communicating by modem, fax, phone line or wireless means may be used to store and operate the suite of software applications. Among the monitoring devices is the Medi-Monitort described in PCT patent application WO98/38909 herein incorporated by reference. The database containing the software applications is the site of the group changes made by the methods of the present invention.

The variety of different medication containers that may be used by the user, that communicate with the information device, could be various size containers. These containers differ in size and functionality, to accommodate the differing preferences and medication regimens of the users. Each container records and date-and-time-stamps the medication compliance of the user, and communicates this data with the information device. The container may also provide other information to the user, such as when to take medication, which medication to take, how many to take, and other special instructions. An example of this type of container could be the Med-eMonitor shown above, which holds the patient's medications, and which could communicate via an infra-red port with a Palm Pilot information device. Another example would be a simple plastic container with numbered medication compartments and compartment lids; provision for the each compartment to hold freestanding medications and/or a standard prescription vial; a clock, memory storage, switches that sense the movements of the compartment lids, and a Blue Tooth wireless communications chip and circuitry. The plastic container would record and date and time stamp all of the medication access by the patient, and communicate this data to the information device. The different sized containers and communications method are demonstrated in attached Drawing #1.

J. Dynamic, Mass Customizable, Interactive Screen and Voice

In an embodiment of the present invention, system 100 is operable for dynamic, mass customizable, interactive screens and voice prompts for selecting medical information preferably, but not necessarily, as described in commonly assigned U.S. patent Ser. No. 60/248,390, filed Nov. 14, 2000, (herein referred to as the '390 application). The disclosure of the '390 application is incorporated herein by reference as though set forth in its entirety.

The present invention describes methods, apparatus, and operating system useful in rapidly linking medical outpatients and their caregivers to medical information that is both specifically relevant to the patients' medical conditions, and scientifically validated. Dynamically generated interactive screens, and/or interactive voice prompts are presented to patients and caregivers to provide hyperlinks to medical information content that is specifically relevant to optimizing the personal health condition, and medical treatment plans of the patients. The screens provide for icons that, when selected, link the patient and/or caregiver to targeted database information to optimize the patients' and caregivers' knowledge base, and improve the health and treatment of the patient. Voice prompts may also be used to access the database information. The dynamically generated screens may be remotely created, modified, or viewed through role-based assignments granted to the patient, their caregivers, their adult children, and/or the medical professionals responsible for the patients' care. In one embodiment of the invention, the icons are arrayed on the screen of a device, and symbolize medications, side effects, adverse drug events, illnesses or diagnoses, medical treatments, decision support algorithms, evidence-based medical treatment recommendations, health promotion activities, patient queries, physical characteristics, physiologic tests, laboratory tests, protein chip data, genomic data, radiographic tests, other biologic measures of the patient, and any other icons that symbolize a link to medical information pertinent to the patients' healthcare, treatment and condition. These icons may be pictorial or symbolic representations, and/or key words. In another embodiment of the invention, key words providing the links can be displayed, highlighted, and embedded in the patient's medication instructions, medical education content, queries, or other information presented to the patient as part of their individualized medical treatment plan. In another embodiment of the invention prearranged key words are entered into a voice recognition system, that then links the user to the desired medical information. The dynamically generated, interactive screens may be utilized by the patient and/or caregiver via a personal computer (PC), personal digital assistant (PDA), wireless information device (WID), cellular telephone, interactive television, or any other device that provides a display screen and links to information contained in one or more databases. The voice prompt method of data access may also make use of voice inputs to these devices. The method has application in linking outpatients and their caregivers to medical databases containing individually-relevant, scientifically validated, medical information that supports the outpatient's specific medical treatment plan or protocol, through remote devices that can assist the outpatient and caregiver in optimizing the outcome of the patient's medical treatment plan over time and space. These outpatients may be managed by one or more medical treatment or clinical research protocols or plans that involve; pharmaceutical drugs, physiologic data, treatment instructions, medical educational content, medication compliance assessment, and health status or quality of life assessment. Links to further information related to these protocols or plans is translated into a set of dynamically generated interactive screens and/or voice prompts that are created based upon the content of the specific protocol or plan for the patient, and are stored in a medical database, and configured, stored and displayed on the remote device used by the patient and/or caregiver. The patient and/or caregiver then interact with the icons on the screen displayed by the remote device, for example by pressing a touch-point represented by the icon, clicking on, or scrolling to and selecting the icon. Each icon selection provides real-time links to information stored in a database in the device itself, or links to other databases via the Internet, direct dial-in, virtual private networks, or other types of connections. The interactive screens may be created by a mass-customization method, whereby groups or populations of patients that share a particular characteristic (e.g. a particular medication, diagnosis, laboratory test result, etc.) are presented identical icons on their devices, linking them to population-based content. In addition, individual patients may have additional icons assigned to them that create links to individually relevant medical content. The same method of linkage can be provided by pre-selected and programmed voice prompts that are entered into the devices. The information provided through the associated links facilitates the support of a patient's complex medical treatment plan by providing the patient and caregiver the opportunity to rapidly locate in real-time patient-specific and population-specific knowledge, to assist patients and their caregivers in optimizing health management. By using the dynamic interactive screen and voice-prompt method, individual patient management goals and improved patient treatment outcomes may be realized by outpatients with chronic and complex conditions, and the caregivers who treat them.

Various electronic devices may be used to monitor and manage medical treatment regimens and protocols for treating a patient's medical condition. These devices communicate one or more of the following: medication schedule and instruction data, medical treatment data, medical education content, patient query data and patient response data, and physiologic data. The devices include a controller for controlling modes of device operation, controlling access to the memory, controlling the communication of treatment data and patient query data on a display or via voice communications means, receiving and processing patient response data, tracking timing, and providing scheduled medication alarm signals. The devices may include soft function keys interfaced with the controller. The soft function keys signal the controller, commanding it to execute different modes of operation of the medical monitoring device. The devices may also provide for scheduled medication alarm signals that alert the user concerning prescribed medications to be taken. The devices may also monitor physiologic conditions, biochemical markers, protein chip data, and other data related to the health of the patient.

While using various devices to monitor outpatients with chronic illnesses, and among clinical drug trial participants, patients often face complicated medical treatment plans or protocols. Despite the miracles provided by an increasing array of modern medical treatments and medications, there is nonetheless a “dark side” created by the growing complexity of these regimens, which provides enormous challenges to patients and their caregivers who must precisely follow the complex medical plan to achieve good health outcomes. For example, patients with congestive heart failure, HIV/AIDS, or other chronic and complex conditions may have to manage well over 100 specific treatment-related-events in a given day. If improperly managed, the patient's illness may worsen, or death might ensue. Properly managed, the patient will have a good outcome and health will be maintained.

Frequently, these patients and their caregivers desire additional information regarding the medical condition and treatment of the condition, and access this information via the Internet. Much of the information accessed via the Web is questionable regarding its scientific accuracy and validity. It may also be quite difficult to rapidly locate information that is most pertinent to the patient's condition and treatment.

Given the infinite number and combinations of potential treatment elements available to include in a medical treatment plan, and the growing complexity of these plans, software-driven databases are used to organize, manage, and communicate the treatment, and monitor the effects of treatment via uploaded information captured by patient monitoring devices. These databases may also include decision support software to guide decision making regarding diagnosis and treatment. The patient may be furnished an interactive information device, and be presented via a display, or via audible means, an automated series of prompted and recorded events to manage and monitor their medical condition. These events may include medical information content that is displayed on the device. The caregiver may also have a device to access medical database information, prescribe drug regimens, capture medical procedure charges and so forth. However, the accessed medical content may be limited in scope, may not contain the level of detail desired by the patient or caregiver, may not be desired by the patient or caregiver, may not be delivered rapidly enough due to the need to search many databases, and may not be optimally tailored for the patient and caregiver in a fashion that promotes the best medical outcome for that patient.

What is missing from the previous methods, yet desirable for patients, medical personnel, caregivers, or family members monitoring and managing outpatients, is the ability to readily and easily access further pertinent medical information in real-time that is specifically desired by the user.

Further, it is desirable that a simple user interface and operating system be constructed such that an infinite number of medical informational elements be converted to a finite number of scientifically validated informational elements, selected via icon-driven links presented to the patient and/or caregiver on the screen of an information device, and/or via voice prompts entered into a device.

Further, it is desirable that the icons may be pictorial or symbolic representations and/or key words related to the data elements that comprise the patient's medical treatment plan or any other aspects of the patient's medical condition and health.

What is also desirable is that the icon-driven screens and/or voice prompts be dynamically generated via rules-based software. The rules-based software formats the icons and screens, and recognizes the correct voice prompts, based upon relationships to the ever-changing data elements that comprise the patient's medical treatment plan, medical condition, physiologic status, biochemical analysis, laboratory test results, and any other elements related to the health of the patient. As the data elements that characterize the patient change, so do the corresponding icons and voice prompts that provide linkage to medical informational content.

What is also desirable is that the icon-driven screens and voice prompts are created and communicated to groups or populations of patients based upon one or more shared characteristics of the group or population, while simultaneously enabling individualized patient icons and voice prompts.

What is also desirable is that a “Site Administrator” designated for the patient will remotely select and pre-approve the icon-driven and voice prompt-driven links and medical content that may be accessed by the patient and caregiver. The icon-driven screen and recognized voice prompts thereby created are downloaded to the patient's and caregiver's devices. The icons are displayed on the screens of the device.

What is also desirable is that the patient or caregiver, through the selection of icons, and/or the vocalization of voice prompts be immediately linked to pertinent medical content. This content may be pre-approved and scientifically validated, or may be unscreened content available over the Internet.

What is also desirable is that the links to medical content be presented as embedded in messages presented to the patient as part of the prompted events comprising the patient's medical treatment plan.

What is also desirable is that each medication, side effect, symptom, illness, diagnosis, adverse drug event, laboratory test result, biological characteristic, related condition, physiologic measure, protein chip data, proteomic data element, genomic data element, or any other element related to the health of the patient be represented by an icon presented on the screen of the patient's device, and/or a voice prompt, enabling the linkage to detailed information regarding that specific data element.

It is therefore the object of the present invention to enable real-time access to information regarding a complex medical condition of a medical outpatient via interactive, icon-driven screens presented to the patient or caregiver on the screen of a remote communications device.

It is a further object of the present invention that the icon-driven screens are displayed as the home screen of a remote communications device.

It a further object of the present invention to enable real-time access to information regarding a complex medical condition of a medical outpatient via interactive voice prompts spoken by the patient or caregiver into the remote communications device.

It is a further object of the present invention that each icon, when selected, or voice prompt, when spoken, provides a hyperlink to medical information that is specifically relevant to the patient's personal health condition and medical treatment plan. The purpose of the medical information is to optimize the patient's and caregiver's knowledge and improve the medical treatment and health of the patient.

It is a further object of the present invention that each icon and voice prompt is related to data elements about the patient, including pharmaceuticals taken by the patient, physiologic parameters, psychological measures, biologic measures, laboratory tests, biochemical analyses, treatment instructions, medical conditions, medication compliance issues, health status or quality of life issues, and any other elements related to the health of the patient.

It is the further object of the present invention that as the data elements regarding the patient change, there will be a corresponding change in the icons displayed on the screen of the remote communications device, and the recognized voice prompts, and a corresponding change in the linked medical content accessed by selecting the icons and/or speaking the prompts.

It is the further object of the present invention that specific icons and voice prompts, and thereby specific medical informational content, are associated with specific groups or populations of patients, and may be assigned to patients within a population or group.

It is the further object of the present invention that the population or group-based icon and voice prompt assignments be supplemented by individually assigned icons and voice prompts that link an individual patient and caregiver to information specifically relevant to them.

It is the further object of the present invention that the icon-driven screens and approved voice prompts be remotely created, modified, and downloaded to the remote device for display on the device, depending upon role-based assignments. These assignments permit different levels of access to, and modification of, the icons and prompts, depending upon the assigned role of the caregiver in the patient's treatment. Role-based assignments determine who has permission to remotely or directly modify the screen-based icons and voice prompts for the patient.

It is the further object of the present invention that the icon-driven screens provide for selection of one or more icons, which, when selected, provide for real-time communications linkages to medical information. The medical information is then presented to the patient or caregiver via downloading to the remote device that is linked to the database containing the medical information. The information may be presented by visual and/or audible communications means. The information may be pre-approved scientifically validated information, or unscreened general information available over the Internet.

It is the further object of the present invention that each of the voice prompts, when spoken, provides for real-time communications linkages to medical information. The medical information is then presented to the patient or caregiver via downloading to the remote device that is linked to the database containing the medical information. The information may be presented by visual and/or audible communications means. The information may be pre-approved scientifically validated information, or unscreened general information available over the Internet.

It is the further object of the present invention that the icons be words that are highlighted and embedded in messages presented to the patient as part of the prompted and recorded events that organize and deliver to the patient the medical treatment plan of the patient.

It is the further object of this invention that the interactive, icon-driven screens and approved voice prompts be able to be remotely created, and that the screens and voice prompts, once created, be remotely transmitted to a series of possible remote devices, including personal digital assistants (PDAs) such as the Palm Pilot®, cellular telephones, pagers, interactive televisions, and custom-manufactured medical monitoring devices such as the Medi-Monitor® System. The remote creation of the interactive screens and voice prompts could be accomplished by accessing the database from the remote patient device itself, a separate personal computer or PDA, a “thin-client” Internet terminal, or other means.

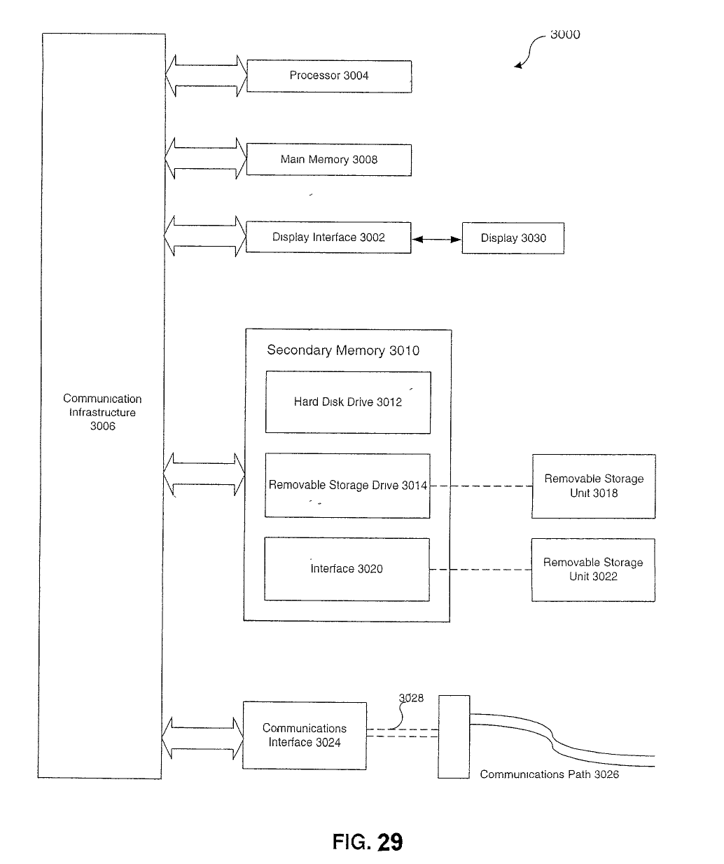

The patient data elements related to the icons displayed on the remote patient device, in one embodiment as illustrated in FIG. 29 or the pre-approved voice prompts may relate to instructions or medical educational content about specific medication; medication dosage; patient physiological measurement, for example weight, blood pressure, pulse rate, glucose level, any antigen level, pH, pO₂, temperature, EKG rhythm, pO2 saturation of the blood, hormone level; any psychological measurement, for example the score based upon standardized or non-standardized tests measuring anxiety, stress, anger, suicidal tendencies, schizophrenic relapse, rapid cycling bipolar relapse or confusion; medical education content related to any disease state or medical condition, for example congestive heart failure, hypertension, angina, circulatory inadequacy or other disease condition of the cardiopulmonary and circulatory systems, specific organ failure, dysfunction of an organ or system or transplanted organ such as asthma for the lungs, kidney failure for the kidney, arteriosclerosis or atherosclerosis of the heart, and transplantation of lung, kidney or heart; class of pathogen, or a specific pathogen; for example instructions or content may be based upon viral infection, or upon a specific viral disease such as HIV/AIDS, hepatitis A,B,C,D,E or G. Similarly, the data elements may be based upon a bacterial disease agent, for example tuberculosis, malaria, cholera and meningitis; a specific microbial agent such as a virus, bacteria, mycotic infection and parasitic infection; or may be based upon the type of pathology involved or the physiological system effected; for example cancer, autoimmune disease, muscular-skeletal system, or pathology of the hematopoetic, circulatory, reproductive, digestive, endocrine or nervous systems.

In each situation, the patients' icon-driven interactive screens and pre-approved voice prompts will be changed based upon changes in the patients' condition, by entering into the individual patient file the new data elements specific to that patient; for example medication type; medication dosage; dietary regimen, etc. In turn each new icon links to new medical information related to the new data element regarding the patient.

The database software may be any functional system, for example Oracle and Microsoft. The remote device may be any communications device such as a Personal Digital Assistant, cellular telephone, interactive pager, interactive television, or proprietary device such as the Med-eMonitor™.

The creation of dynamic, mass-customizable, interactive screens and voice prompts can be accomplished utilizing a standard ODBC compliant database, for example Oracle 8i. The database may also be an Internet portal, that links the patient and caregiver via the icons and voice prompts to Internet-accessible databases containing the desired medical content. The business logic rules convert the data elements in the patient configuration file into representative screen icons and permitted voice prompts, as the configuration file contains all the data elements of the patient's treatment plan. An example of an Internet-accessible Oracle 8I database, that contains all the data elements of the patient's treatment plan, can be found at www.med-emonitor.com. The example shown below is illustrative of the structure of the med-emonitor.com site, and should in no way be construed as limiting the invention.

Any monitoring device capable of communicating by modem, fax, phone line or wireless means may be used to present the icon-driven screens that link to the database. Among the monitoring devices is the Medi-Monitor® described in PCT patent application WO98/38909 herein incorporated by reference. The database containing the patient information and protocols is the site of the medical treatment plans made by the methods of the present invention. The database incorporating the treatment plan then uploads the icon-driven screens to each patient's and caregiver's information device.

For example, using the above method, medical information related to a complex treatment regimen for a heart failure patient may be accessed via the icons shown below and in FIG. 29. Heart failure patients may typically require the following data elements in their medical treatment plan, with information about each data element to be communicated at a particular time: a medication regimen comprised of a beta-blocker, ACE inhibitor, diuretic, and Digoxin; dosing instructions for each medication; a description of each medication and rationale for taking the medication; what the medication looks like along with its color; daily measures of weight, blood pressure, pulse, and blood oxygen levels; a salt restricted, low fat, low calorie diet; an exercise program tailored to their specific level of heart failure; questionnaires that assess their shortness of breath, chest pain, energy level, and swelling of the legs; information updates about new drugs used to treat the condition; information about when to call the doctor with specific complaints or side effects or adverse drug reactions; and other information.

An example of an icon-driven screen for the heart failure patient is shown below. The meanings of the icons, and the links they provide, are as follows, from left to right:

| | | | |
|---|---|---|---|
| Row 1: | EKG Results | Med-eMonitor | Laboratory Tests |
| Medication | | | |
| Row 2: | Risk Emergency | Ambulance | Blood Pressure |
| Mortality | Care | | |
| Row 3: Your | Contact Doctor | Contact Nurse | Health Risk |
| Bill | | | Factors |

An example of highlighted text that is part of the patient's treatment regimen, embodied in medication instructions presented to the patient, that provides links to medical information, is shown below in bold print:

Drugname=“CELLCEPT 500 mg”

Takenwith=“To Prevent Transplant Rejection. Do not take antacids at the same time you take Cellcept”

PatEducation=“Cellcept suppresses T and B Lymphocytes to prevent transplant rejection. Call Dr. Johnson at 202-784-3700 if you have blood in your stool”

Refillto=28

Successtext=“Good job. Cellcept helps prevent organ rejection”

Failuretext=“Not Complying with your prescription may cause acute rejection of your transplant”

IV. Software and Hardware Embodiments

The present invention (e.g., system 100, or any part thereof) can be implemented using hardware, software or a combination thereof and can be implemented in one or more computer systems or other processing systems. In fact, in an embodiment, the invention is directed toward one or more computer systems capable of carrying out the functionality described herein.

Referring to FIG. 30, an example computer system 3000 useful in implementing the present invention is shown. The computer system 3000 includes one or more processors, such as processor 3004. The processor 3004 is connected to a communication infrastructure 3006 (e.g., a communications bus, cross-over bar, or network). Various software embodiments are described in terms of this exemplary computer system. After reading this description, it will become apparent to a person skilled in the relevant art(s) how to implement the invention using other computer systems and/or computer architectures.

Computer system 3000 can include a display interface 3002 that forwards graphics, text, and other data from the communication infrastructure 3006 (or from a frame buffer not shown) for display on the display unit 3030.

Computer system 3000 also includes a main memory 3008, preferably random access memory (RAM), and can also include a secondary memory 3010. The secondary memory 3010 can include, for example, a hard disk drive 3012 and/or a removable storage drive 3014, representing a floppy disk drive, a magnetic tape drive, an optical disk drive, etc. The removable storage drive 3014 reads from and/or writes to a removable storage unit 3018 in a well-known manner. Removable storage unit 3018, represents a floppy disk, magnetic tape, optical disk, etc. which is read by and written to removable storage drive 3014. As will be appreciated, the removable storage unit 3018 includes a computer usable storage medium having stored therein computer software and/or data

In alternative embodiments, secondary memory 3010 can include other similar means for allowing computer programs or other instructions to be loaded into computer system 3000. Such means can include, for example, a removable storage unit 3022 and an interface 3020. Examples of such can include a program cartridge and cartridge interface (such as that found in video game devices), a removable memory chip (such as an EPROM, or PROM) and associated socket, and other removable storage units 3022 and interfaces 3020 which allow software and data to be transferred from the removable storage unit 3022 to computer system 3000.

Computer system 3000 can also include a communications interface 3024. Communications interface 3024 allows software and data to be transferred between computer system 3000 and external devices. Examples of communications interface 3024 can include a modem, a network interface (such as an Ethernet card), a communications port, a PCMCIA slot and card, etc. Software and data transferred via communications interface 3024 are in the form of signals 3028 which can be electronic, electromagnetic, optical or other signals capable of being received by communications interface 3024. These signals 3028 are provided to communications interface 3024 via a communications path (i.e., channel) 3026. This channel 3026 carries signals 3028 and can be implemented using wire or cable, fiber optics, a phone line, a cellular phone link, an RF link and other communications channels.

In this document, the terms “computer program medium” and “computer usable medium” are used to generally refer to media such as removable storage drive 3014, a hard disk installed in hard disk drive 3012, and signals 3028. These computer program products are means for providing software to computer system 3000. The invention is directed to such computer program products.

Computer programs (also called computer control logic) are stored in main memory 3008 and/or secondary memory 3010. Computer programs can also be received via communications interface 3024. Such computer programs, when executed, enable the computer system 3000 to perform the features of the present invention as discussed herein. In particular, the computer programs, when executed, enable the processor 3004 to perform the features of the present invention. Accordingly, such computer programs represent controllers of the computer system 3000.

In an embodiment where the invention is implemented using software, the software can be stored in a computer program product and loaded into computer system 3000 using removable storage drive 3014, hard drive 3012 or communications interface 3024. The control logic (software), when executed by the processor 3004, causes the processor 3004 to perform the functions of the invention as described herein.

In another embodiment, the invention is implemented primarily in hardware using, for example, hardware components such as application specific integrated circuits (ASICs). Implementation of the hardware state machine so as to perform the functions described herein will be apparent to persons skilled in the relevant art(s).

In yet another embodiment, the invention is implemented using a combination of both hardware and software.

K. Analysis and Correlation With Real-Time Compliance

In an embodiment of the present invention, system 100 provides self-selected, synchronized, database-linked medical monitors to outpatients and their caregivers preferably, but not necessarily, as described in commonly assigned U.S. patent Ser. No. 60/227,785, filed Aug. 25, 2000, (herein referred to as the '785 application). The disclosure of the '785 application is incorporated herein by reference as though set forth in its entirety.

Medication is widely regarded as the most cost-effective medical means of treating patients. Prior to and subsequent to bringing a medication onto the market, the pharmaceutical manufacturer must subject the drug compound to rigorous pre-clinical and clinical tests, to determine its safety, efficacy, and costs. These tests are conducted in clinical drug trials, wherein the compound is first tested in animals, then small populations of humans, then in larger populations of humans. First the safety (incidence and severity of side effects) of the drug must be established. Then preliminary data on effectiveness (efficacy) must be established. These early safety and efficacy trials are conducted in small numbers of human study participants. Then larger trials, at times at multiple different pharmaceutical dosing levels, further define the safety and efficacy profile for the drug compound at particular doses. The average cost of bringing a new drug to market is $500,000,000, and takes an average of seven years. Each day of delay in bringing the drug onto the market costs the manufacturer $1,000,000 to $10,000,000 in lost sales, and reduces the post-market life of the drug's patent. Once the drug is on the market, post-market surveillance studies are used to better understand safety, efficacy, the prevalence of side effects, and drug interactions with other drug compounds in combination with the new drug being studied. In addition, new techniques of analyzing the human genome (genomics), human protein structure and function (proteomics), and the physical expression of the genes (phenotyping) are being used to help determine the different responses of genetically and phenotypically different populations of patients to pharmaceutical agents. Various methods may be used to analyze the effectiveness, cost, and adverse effects of medication. Currently these methods are very labor intensive, and have limited technology integrated into the clinical drug trial system. Many of the systems are paper-based, and rely on retrospective data and written patient diaries (Source: “epharma, Accelerating Clinical Trials and Enhancing Details,” Friedman Billings Ramsey Healthcare Industry Analysis, August 2000).

Recently, various technologies have been introduced into the clinical drug trial process, including electronic devices that may be used to monitor and manage medical treatment regimens and protocols for treating a patient's medical condition and monitoring the patient's outcomes. These devices communicate one or more of the following: medication schedule and instruction data, medical treatment data, medical education content, patient query data and patient response data, and physiologic data. The devices include a controller for controlling modes of device operation, controlling access to the memory, controlling the communication of treatment data and patient query data on a display or via voice communications means, receiving and processing patient response data, tracking timing, and providing scheduled medication alarm signals. The devices may include soft function keys interfaced with the controller. The soft function keys signal the controller, commanding it to execute different modes of operation of the medical monitoring device. The devices may also provide for scheduled medication alarm signals that alert the user concerning prescribed medications to be taken.

However, what is missing from current methods of pharmacoeconomic analysis in clinical drug trials is a medical database that contains the following real-time data captured from patients: medication compliance for one or more medications taken by the patient, patient answers to health status and quality-of-life questionnaires, patient physiologic data, and patient laboratory data.

What is also missing is the ability to combine this data with other database data such as genomic, proteomic, phenotypic, economic, and other healthcare related data, for further data analysis.

What is also missing is the statistical analysis and data mining of part or all of the data to include the method of correlating patient medication dosing patterns of one or more drugs ingested by the patient, with various other measures of clinical and economic outcomes of the patients, by translating a complex medical treatment plan of a medical outpatient into a sequential series of automated, prompt and record events presented over time; to medical monitors used by a single patient. The medical monitors capture data on medication compliance, health status, quality-of-life, physiologic status (e.g. blood pressure, EKG, pO2, pulse rate, weight, pulmonary function, etc.), and various measures of blood, serum, urine, and other laboratory tests. The medical monitors are linked to a database that updates and synchronizes the information presented to all of the monitors, and captures information from all of the monitors. Individual patient medical treatment plans may be remotely created, modified, or viewed in the database; and in turn are transformed by business logic rules and a communications system into prompted and recorded events that are delivered to the patient by the remote monitors.

What is also missing form the present methods is the ability to combine the patient monitoring data with genomic, proteomic, and physiologic data for analysis and data mining purposes, to predict the responses of subpopulations of patients to a given drug or combination of drugs, in different doses and combinations, based upon the specific genetic and physical attributes of these subpopulations.

It is therefore an object of the present invention to enter into a medical database the following real-time data captured from patients: medication compliance for one or more medications taken by the patient, patient answers to health status and quality-of-life questionnaires, patient physiologic data, and patient laboratory data.

It is a further object of the present invention to provide the ability to combine this data with other database data such as genomic, proteomic, phenotypic, economic, and other healthcare related data, for further data analysis.

It is a further object of the present invention that the statistical analysis and data mining of part or all of the data will include the method of correlating patient medication dosing patterns of one or more drugs ingested by the patient, with various other measures of clinical and economic outcomes of the patients, by translating a complex medical treatment plan of a medical outpatient into a sequential series of automated, prompt and record events presented over time; to medical monitors used by a single patient. The medical monitors capture data on medication compliance, health status, quality-of-life, physiologic status (e.g. blood pressure, EKG, pO2, pulse rate, weight, pulmonary function, etc.), and various measures of blood, serum, urine, and other laboratory tests.

It is a further object of the present invention that combination of the patient monitoring data with genomic, proteomic, and physiologic data for analysis and data mining purposes will predict the responses of subpopulations of patients to a given drug or combination of drugs, in different doses and combinations, based upon the specific genetic and physical attributes of these subpopulations.

It is a further object of the present invention that the knowledge thereby gained from the data analysis and mining can be used to: adjust the dosing recommendations of one or more medications to improve clinical outcome, reduce the frequency of side-effects, reduce the severity of side-effects, eliminate adverse drug events, reduce or eliminate drug-interactions, and determine the most cost-effective means of using drugs to improve the health of specific populations of patients.

The data elements that comprise the medical data entered into the database may relate to instructions or medical educational content about specific medication; medication dosage; patient physiological measurement, for example weight, blood pressure, pulse rate, glucose level, any antigen level, pH, pO_{2,} temperature, EKG rhythm, pO2 saturation of the blood, hormone level; cell surface receptors; serum proteins; DNA data; Protein data; any psychological measurement, for example the score based upon standardized or non-standardized tests measuring anxiety, stress, anger, suicidal tendencies, schizophrenic relapse, rapid cycling bipolar relapse or confusion; medical education content related to any disease state or medical condition, for example congestive heart failure, hypertension, angina, circulatory inadequacy or other disease condition of the cardiopulmonary and circulatory systems, specific organ failure, dysfunction of an organ or system or transplanted organ such as asthma for the lungs, kidney failure for the kidney, arteriosclerosis or atherosclerosis of the heart, and transplantation of lung, kidney or heart; class of pathogen, or a specific pathogen; for example instructions or content may be based upon viral infection, or upon a specific viral disease such as HIV/AIDS, hepatitis A,B,C,D,E or G. Similarly, the instructions or content comprising the data elements may be based upon a bacterial disease agent, for example tuberculosis, malaria, cholera and meningitis; a specific microbial agent such as a virus, bacteria, mycotic infection and parasitic infection; or may be based upon the type of disease or pathology involved or the physiological system effected; for example cancer, autoimmune disease, muscular-skeletal system, or pathology of the hematopoetic, circulatory, reproductive, dermatologic, digestive, endocrine or nervous systems.

The database software may be any functional database system, for example Oracle and Microsoft. The DNA profile detection may be provided by any DNA Chip System, such as that provided by Affymatrix Corporation. The serum protein and cell surface receptor data may be provided by any Protein Chip System, such as that provided by Affymatrix Corporation. The medical data statistical analysis and data mining functions can be performed by any functional system, such as that provided by Medical Internet Solutions, Inc. The business logic rules may be any functional system, such as TEOCO Corporation's PageNgine, or DREAM (Dynamic Rapid Enterprise Application Method). The software that synchronizes the database with the remote patient monitoring devices may be any functional system, such as Aether Corporation's ScoutSync technology. The remote device may be any communications device such as a Personal Digital Assistant such as Palm Pilot®, cellular telephone, interactive pager, interactive television, or proprietary device such as the Med-eMonitor™.

The medical database used to store and analyze the real-time data captured from patients; medication compliance for one or more medications taken by the patient, patient answers to health status and quality-of-life questionnaires, patient physiologic data, patient laboratory data, genomic, proteomic, phenotypic, economic, and other healthcare related data can be accomplished utilizing a standard ODBC (Object-oriented database compliant database). The example shown, written in Microsoft SQL Server 7, is illustrative in the FIGURES, and should in no way be construed as limiting the invention.

L. Transaction-Based Monitoring

In an embodiment of the present invention, system 100 provides self-selected, synchronized, database-linked medical monitors to outpatients and their caregivers preferably, but not necessarily, as described in commonly assigned U.S. patent Ser. No. 60/241,672, filed Oct. 19, 2000, (herein referred to as the '672 application). The disclosure of the '672 application is incorporated herein by reference as though set forth in its entirety.

Various electronic devices may be used to monitor and manage medical treatment regimens and protocols for treating a patient's medical condition, and for managing other aspects of a person's work and personal life. These devices communicate one or more of the following: for medical applications it may include medication schedule and instruction data, medical treatment data, medical education content, patient query data and patient response data, and physiologic data; for non-medical applications it may include Internet content, work-related instructions, instant message instructions, sequences of procedures and actions, and other information that assists the user in day-to-day life management. The devices include a controller for controlling modes of device operation, controlling access to the memory, controlling the communication of treatment data and patient query data on a display or via voice communications means, receiving and processing patient response data, tracking timing, and providing scheduled medication alarm signals and other instruction-related signals. The devices may include soft function keys interfaced with the controller. The soft function keys signal the controller, commanding it to execute different modes of operation of the device. The devices may also provide for scheduled medication alarm signals that alert the user concerning prescribed medications to be taken.

Various technologic systems exist for using various devices to monitor outpatients with chronic illnesses in clinical practice, and in clinical drug trials; monitor customer Internet access, and monitor other customer behaviors and responses. As well, various payment transaction terms and business models govern the provision of these services. One such business model is the so-called Application Services Provider or “ASP.” Under the ASP model, a customer contracts with the ASP to provide a variety of database, communications, and transaction services that link peripheral devices with a central database to provide an “enterprise-wide” information system. Under the ASP arrangement the customer does not purchase database software and does not maintain the database or information system, but instead “leases” the services on a monthly, annual, or per-transaction basis. The ASP provider in turn provides access to the system, database services, hardware and software support, hardware and software maintenance, software and system upgrades, help desk support, and other services. The advantages to the customer are many, as the customer is relieved from the responsibility and costs of setting up and maintaining such a system, and upgrading it, which would involve expertise frequently outside of the customer's capabilities. The advantages to the ASP provider are a recurring revenue stream that is based-upon creating, maintaining, and upgrading an information system and remote access to it, which becomes the core capability of the ASP.

In the healthcare industry, given the infinite number and combinations of potential treatment elements available to include in a medical treatment plan, and the growing complexity of these plans, software-driven databases are used to organize, manage, and communicate the treatment. These databases frequently communicate with a series of remote devices used by the patient or caregiver. Each remote device is linked to the database, and has particular differences in functionality. The devices relate to the fields of “Telemedicine” and “eHealth.” At the present time these devices and databases are delivered under a variety of payment methods including daily or monthly fees per device in use, global fees per time period or clinical trial, device sales independent of database linkage, along with or independent of software or hardware sales and installation at the customer site, or via ASP services.

In addition, increasingly, these services are provided by two or more companies that comprise a “value chain,” with each providing a different service component of the ASP services, and charging a fee for their portion of the service provision.

What is missing from the previous methods of providing and managing these services, yet desirable for ASP companies, patients, medical personnel, caregivers, or family members monitoring and managing outpatients, and other users of remote information devices, is the ability to readily and easily convert the financial transaction terms that define monitoring and reporting service agreements into an automated, transaction-based, monitoring and accounting system. Further, it is desirable that the information system, database, and communicating devices be constructed in such a way that in non-medical applications the device user, and in medical applications the patient, caregiver, or medical personnel, can use multiple devices; one or more of which may be provided under separate financial transaction terms, that are accounted for by the accounting system. Further, it is desirable that one or more databases be repositories for one or more of the patient's treatment plan, and for the patient's responses to treatment as recorded by the medical devices, such that the entire treatment plan can be delivered and monitored through multiple devices. Further it is desirable that the database be the central repository for prescription orders by physicians, patient electronic medical record data, laboratory data, radiologic and other medical test data, and any clinical data related to the patient's condition and care. Further, it is desirable that the accounting system calculate transaction-based sales commissions and payroll bonuses, license fees and royalties, and vendor payments and invoices, based upon device usage under the ASP model. Further it is desirable that the accounting system compare customer payments with financial transaction terms, to determine whether ASP monitoring services will be continued uninterrupted, or whether a warning message of impending interruption is delivered, or whether the ASP services to patients, medical personnel or caregivers themselves are discontinued. Further it is desirable that a customer relations component of the system query users regarding their satisfaction levels, provide for submission by them of a wish list of new functions and features of the device and service, provide confirmation of system communications integrity, provide rapid locator services to locate professionals to assist the user, provide for peer-group determined chat rooms and instant messages, and provide for rank-order based triaging based upon the criticality of data entered by the user.

It is therefore and object of the present invention that the following data is inputted into a database system: financial transaction terms that govern the terms and conditions of ASP services used by a customer; purchase/lease/rental rates for remote devices that communicate with the database; days of device use by individual, group and site; commission structure by salesperson; structure of license and royalty payments; financial terms of vendor relationships; data inputted into each device by the device user; payments by the customers; other database services purchased by users; prescription drug ordering data, and patient clinical monitoring data.

It is the further object of the present invention that the database provides business logic rules that provide a correlation or lack of correlation between transaction terms versus actual payments, financial calculations, financial report algorithms, and a determination of ASP service continuation versus discontinuation.

It is a further object of the present invention that the database provide customer relations software that performs the following: user billing status query information including current, last, and next bills; due dates of bills, detailed billing analysis and answers to billing questions including billed-for items and payments received; customer satisfaction information collected on the device including satisfaction questionnaires related to the device and the service, wish list for new device functions and services, and rank-ordering of wishes by population; instant messaging triage by message urgency, with voice and/or visually communicated responses; disease, drug, and group-specific chat rooms; customer query capability regarding patient record data; manual and automatic professional locator functions to locate and match professionals with specific user locations, disease states, and medications used; user peer-group rankings of these professionals; chat room monitoring by Help Desk Personnel to provide assistance; test routines that test the integrity of the communication between the database and the remote device; rank-ordering of the criticality of the data being received, with triage and outbound messaging to the appropriate recipient of the data; and other functions.

It is the further object of the present invention that the database provides a series of outputs that include commission bonus and payroll calculations; license fees and royalty payments; invoices or payment amounts to vendors, user charge and payment data by individual, group and site; system usage data by individual device, groups of devices, or sites of devices; continuation versus discontinuation of services; warning messages of impending service discontinuation; and approval of prescription orders and the vending of medications to patients.

It is a further object of the present invention to translate a complex medical treatment plan of a medical outpatient into a sequential series of automated, real-time, time-and-event-driven, prompt and record events presented over time to the patient via multiple monitoring and communication devices that may be remotely located, facilitating mobile information transfer to the patient.

It is a further object of the present invention that the medical protocols may reside in the database, and be transferred into the memories of the remote devices. The remote devices contain software or firmware that translates the stored medical protocol into a sequential series of prompt and record communication events over time, that are then delivered to the and captured from the patient via the devices.

It is a further object of the present invention that the medical protocols may reside in the database, and be directly transferred as a sequential series of automated, real-time, time-and-event-driven signals to the multiple remote devices. The devices then convert the signals into real-time, prompt-and-record communication events that are delivered to and captured from the patient.

It is a further object of the present invention that medical data may be delivered to the medical personnel or caregivers in the form of visual communications, audible communications (e.g. voice or music), or some combination thereof, and that the caregivers or medical personnel can enter data into the database related to the patient's condition, laboratory values, prescription drug orders, or any other element related to the patient's medical treatment.

It is a further object of the present invention that these events and reports may then be communicated to patient monitoring devices, and/or caregiver and medical personnel devices, such as a “smart phone” cellular telephone, a Personal Digital Assistant (PDA) such as the Palm Pilot®, or a proprietary device such as the Med-eMonitor Medication Dispenser; and/or various types of physiologic monitors that measure the patient's physical responses to treatment.

It is a further object of this invention that the patient, caregiver, or medical personnel can select one or more of the communicating devices at a particular time based upon treatment plan or lifestyle considerations, and the database will communicate the correct information to the particular device at the correct time, based upon the patient's medical treatment plan, results of treatment, results of laboratory or testing data, or other medically-related data.

It is a further object of this invention that the terms of the financial transactions be correlated with services provided to specific patient-selected, caregiver-selected, and medical personnel-selected devices that permit the devices to access information from the database only if payments for the services are consistent with the terms of the financial transactions.

It is a further object of this invention that updated information services be made available to the medical personnel, patient or caregiver over multiple devices available to each user of the information system. The information services include, but are not limited to; compliance monitoring, physiologic monitoring; laboratory test results; personal emergency response monitoring; patient counseling; patient performance reports that indicate how well the patient is following the medical treatment plan; patient peer-group-based performance/outcomes reports, drug-interaction reports (prescription, over-the-counter, herbal, nutriceutical, vitamin, etc.), ordering prescription drugs, and other information services and reports. The database business logic and rules will determine for each device used by the user whether the payment terms of use have been met.

The information could be accessed from the database by the multiple devices via an Internet, Extranet, Intranet, direct dial-in, wireless broadcast, faxed, mailed, or telephoned means of communication. In this way each user can select the particular information device best suited to their particular needs at the time. These devices could include a laptop PC, desktop PC, Personal Digital Assistant (PDA), smart phone, or other computing device.

M. System For Providing Management

In an embodiment of the present invention, a system is configurable for providing real-time management, pharmacy benefits management, inventory management, and pharmaceutical manufacturing management. As is described in commonly assigned U.S. patent Ser. No. 60/266,430, filed Feb. 5, 2001, (herein referred to as the '430 application). The disclosure of the '430 application is incorporated herein by reference as though set forth in its entirety.

While using various devices to monitor outpatients with chronic illnesses, and among clinical drug trial participants, patients often face complicated medical treatment protocols or regimens. These protocols require the patient to carry out a detailed series of events, in a sequential fashion throughout the day, related to taking medication, following other instructions (e.g. taking their blood pressure or blood glucose levels and transmitting these values to a remote database), answering questions that assess their health status, and other events that need to be prompted and recorded. Some of these events and information are population-specific, and apply to entire populations of patients with specific diseases. Other events and information apply to sub-populations of patients, and still other events are specific to the individual patients themselves and represent specific elements of the protocol that apply only to them and to no other patient.

What is missing from the previous methods, yet desirable for the medical personnel, caregivers, or family members monitoring and managing outpatients, is the ability to readily and easily mass customize the information provided to those patients, and the information to-be-captured from those patients. Such mass customization would enable patients and those who monitor them to insure that patients get the right information at the right time, to sequentially follow a complex medical protocol, and in-turn that the patients get the benefit of both population-based and individualized monitoring and treatment.

It is therefore the object of the present invention to enable those medical personnel and caring family members to select and provide information to populations of patients, for example educational content and disease-specific questionnaires to a population of patients with congestive heart failure; to sub-populations of patients, for example patients with congestive heart failure on a specific medication or combinations of medications; and in addition be able to provide each individual patient with information that is specific to them, for example medication dosing instructions, and individualized educational content and questionnaires.

It is the further object of this invention that the provision of this mass customized information be enabled by a remotely accessible database that contains the patient files. Each patient file is constructed in such a fashion that it may be assigned population-specific data, for example educational content and questionnaires that relate to an entire population or sub-population of patients; and simultaneously provide for the ability to enter into the patient file specific educational content for that specific patient (e.g. nutritional information), specific medication dosing instructions, specific queries for the patient regarding their health status and quality-of-life, and any other information of value to the management or monitoring of the patient or population of patients.

It is the further object of this invention that the patient file may receive information that is retrieved from other databases; via the use of search engines that search other databases for content that is specific to a population or sub-population of patients. This search and retrieve function can be applied to medical education content, self-management instructions, medication-specific dosing or side-effect information, self-diagnosis algorithms or questionnaires, drug-interaction information and warnings, or any other information that will improve the medical outcome of the patient by delivering it to a specific population of patients.

It is the further object of this invention that the mass customized database, once created and placed into use, can be imported into other pre-existing databases that are used to monitor and manage patients, or used for statistical analysis in clinical trials, such that these other database users benefit from the addition of the mass customization feature and data to their particular database application.

It is the further object of this invention that the patient file be able to be remotely created, by accessing the database via direct dial-in or Internet access; and that the patient file, once created, be remotely transmitted to a series of possible patient monitors, including personal digital assistants (PDAs) such as the Palm Pilot®, cellular telephones, pagers, interactive televisions, and custom-manufactured medical monitoring devices such as the Medi-Monitor® System. The remote creation of the patient file could be accomplished by accessing the database from the patient monitor itself, a separate personal computer or PDA, a “thin-client” Internet terminal, or other means.

Each monitor may, but need not, have associated with it medication compartments that communicate with the monitor or clip onto it, such that the compartments have sensors that sense when the medication is being removed, and communicate this information to the memory of the device, or directly to the database.

Once the patient file is transmitted to these monitoring devices, the monitoring device firmware converts the file into a series of messages and queries to assist the patient in following the protocol, by prompting and monitoring a series of events throughout the day. In the alternative, a wireless signal can carry the application software and the patient file for direct insertion into the wireless device. The patient then interacts with the monitoring devices, which in turn communicate the collected patient information back to the database for report-generation to the medical personnel, caregivers and family members who are monitoring and managing the patients. New mass customization of the patient files can then take place based upon the collected data from the patient. In this fashion a complex protocol that requires a sequential set of actions and monitoring activities can be managed.

The sub-populations or groups of patients may be any group of patients that share one or more common characteristic that may effect or modify their medical condition or treatment protocols. Among the groups to consider are defined by age; gender; occupation; disease state; medical history event; medication category; specific medication; medication dosage; patient physiological measurement, for example weight, blood pressure, pulse rate, glucose level, any antigen level, pH, pO₂, temperature, EKG rhythm, pO2 saturation of the blood, hormone level; or any psychological measurement, for example the score based upon standardized or non-standardized tests measuring anxiety, stress, anger, suicidal tendencies, schizophrenic relapse, rapid cycling bipolar relapse or confusion. The groups may be age, gender, race, national origin, geographic location related in combination with a medical condition. The group may be based upon the same or similar disease state or medical condition, for example congestive heart failure, hypertension, angina, circulatory inadequacy or other disease condition of the cardiopulmonary and circulatory systems. The group may be based upon specific organ failure or dysfunction or upon a transplanted organ, for example asthma for the lungs, kidney failure for the kidney, arteriosclerosis or atherosclerosis of the heart, and transplantation of lung, kidney or heart. The group may be passed upon class of pathogen, or a specific pathogen. For example the group may be based upon viral infection, or upon a specific viral disease such as HIV/AIDS, hepatitis A,B,C,D,E or G. Similarly, the group may be based upon a bacterial disease agent, for example tuberculosis, malaria, cholera and meningitis. Groups may be defined by a specific type of microbial agent such as a virus, bacteria, mycotic infection and parasitic infection. The group may be based upon the type of pathology involved or the physiological system effected; for example cancer, autoimmune disease, muscular-skeletal system, or pathology of the hemopoetic, circulatory, reproductive, digestive, endocrine or nervous systems.

The groups may be based upon two or more characteristics as parameters for defining the group; for example HIV/AIDS and immune function, liver dysfunction and hepatitis, or bone degeneration and arthritis. The groups may be based upon any patient parameter available to the medical database, either directly in the database or accessible through linkage to another database or databases, and associated with a specific medical condition. Among the data in the database are name of patient's physician, physician specialty, hospital, insurance company, type of insurance, diagnosis, length of illness, family history or previous medical history. The group may be based upon the type of clinical setting, for example hospital, emergency clinics, physician's office, institutional setting or home. The group may be based upon the name of the corporation, organization, physician, clinical research organization, pharmaceutical company, case worker or sponsor.

In each situation, the patients' medical protocols will be changed based upon the patients' inclusion in such a group. The change submitted to the group database files will modify each patient's medical protocol in the same manner. Each individual patient may be a member of may different sub-groups, each of which may have a change entered into the database. The changed patient protocol is then uploaded to the monitoring device. Such uploading may be immediate, or it may be at the next scheduled uploading event. Notice of the uploading of a change is made through the monitoring device to alert the patient to the change in protocol.

In addition each individual patient's protocol may be modified in the database by entering into the individual patient file the elements of a medical protocol specific to that patient; for example medication type; medication dosage; dietary regimen; specific reminders, such as when to obtain a medication refill, when to call the doctor; and any algorithm-driven events that are based upon data inputted by the patient into the remote monitoring device, for example instructing the patient to dial “911” if they are having chest pain and have taken too much bronchodilator medication.

The database software may be provided by any operating system, for example those provided by Oracle and Microsoft.

The mass customization of the patient files, which are then translated into operating routines in remote monitors, can be accomplished utilizing a standard ODBC compliant database. The examples, written in Microsoft SQL Server 7, are illustrative, and should in no way be construed as limiting the invention. The FIGURES issustrated are screens used to collect data from patients, healthcare providers, clinicians and administrators regarding database parameters. These screens are present to illustrate one of the methods used in the present invention to populate the database with patient characteristics that may be used to create specific groups in later modifying the information in the database.

As illustrated in the above database entry fields, the goal of mass customization of patient protocols in clinical drug trials and in outpatient medical management is facilitated. The addition of pull-down menus to the fields, that populate the fields with standardized questionnaires and educational content further simplifies the mass customization method. Other fields are then individually populated with patient-specific content to complete the patient's medical protocol design.

The database application of the mass customization system, in this case provided in a Microsoft SQL Server 7 database, can be converted into configuration files that are downloaded via modem, cable, or wireless means into the patient monitors. The files are translated into routines that convert the medical protocol structure into a series of prompt and record events. In this way the patients get the benefit of both population-based as well as individualized assessment and/or treatment protocols on a real-time basis.

Once the caregivers populate the data fields in the database, which converts these fields into configuration files that are downloaded into the monitors, the patient management protocols can be instantaneously updated. Through this method, the caregivers need not develop new software each time they want to change the database or patient protocols, and can rapidly take advantage of newly discovered population-based medical evidence to be communicated to patients, to give patients the advantages of up-to-the-minute medical knowledge without any delay caused by reprogramming or creating new software.

Alarm-based educational content messages, alarm-based treatment instructions, and population-based questionnaires can be assigned to specific groups of patients as previously disclosed. In addition, each individual patient file can be programmed for specific medication instructions by populating the appropriate fields.

In addition the patient file may receive information that is retrieved from other databases; via the use of search engines that search other databases for content that is specific to a population or sub-population of patients, or to an individual patient. The search engine can carry our its search based upon a single characteristic of a population, two or more simultaneous characteristics of a sub-population, or a combination of specific characteristics that are particular to an individual patient. This search and retrieve function can be applied to medical education content, self-management instructions, medication-specific dosing or side-effect information, self-diagnosis algorithms or questionnaires, drug-interaction information and warnings, or any other information that will improve the medical outcome of the patient by delivering it to a specific population of patients.

Thus a mass customization of patient management protocols is facilitated by the above database design; and implemented by downloading into monitors that translate the databased data into protocolized routines.

Any monitoring device capable of communicating by modem, fax, phone line or wireless means may be used to collect the patient data that is communicated to the database for storage and for display. Among the monitoring devices is the Medi-Monitor® described in PCT patent application WO98/38909 herein incorporated by reference. In the present invention, the Medi-Monitor® described may be further modified by placing the software and key functions and related software is a device not containing medication compartments. The medication compartments may be present in a separate associated device that contains compartments that indicate which compartment contains the medication and communicates by wireless means with the monitor containing the screen. Thus one type of Medi-Monitor® may have a separate component that communicates by wireless means with the monitor portion and may contain from 2 to 25 different medication compartments, more preferably 2-15 medication compartments, and most preferably 3-10 medication compartments.

The database containing the patient information and protocols is the site of the group changes made by the methods of the present invention. The database incorporating the group changes then uploads the modified protocol to each patients monitoring device for use by the patient as directed.

V. Conclusion

While various embodiments of the present invention have been described above, it should be understood that they have been presented by way of example, and not limitation. It will be apparent to persons skilled in the relevant art(s) that various changes in form and detail can be made therein without departing from the spirit and scope of the invention. Thus, the present invention should not be limited by any of the above described exemplary embodiments, but should be defined only in accordance with the following claims and their equivalents.

What is claimed is:

1. A method for mass-modification of protocols in an information system, comprising the steps of: determining target characteristics pertaining to target patients having protocol information capable of being updated; comparing said target characteristics with source characteristics contained in profiles of source patients to find a match between said target characteristics and said source characteristics;
updating protocol information of said target patients, said protocol information of target patients being copied from said profiles of said source patients; and transmitting the updated protocol information to a corresponding patient monitoring device.

2. The method of claim 1, further comprising the step of instructing said corresponding patient monitoring device to convert the updated protocol information into a series of queries, whereby responses from said queries are communicated to a remote device for report generation to medical personnel.

3. The method of claim 1, further comprising the step of communicating said responses to the information system.

4. The method of claim 1, further comprising the step of converting said protocol information of said target patients into a series of messages.

5. The method of claim 3, further comprising the step of requiring said target patients to carry out a detailed series of events related to taking medication.

6. The method of claim 3, further comprising the step of requiring said target patients to follow a series of instructions.

7. The method of claim 3, further comprising the step of accessing health status of said target patient.

8. The method of claim 3, further comprising the step of relating said messages to any other information of value in monitoring of said target patient.

9. The method of claim 1, further comprising the step of selecting said source characteristics from the group comprising:
age, gender, occupation, disease state, medical history event, medication categories, medication dosages, physiological measurement, psychological measurement, geographical location, organ failure, clinical setting, pharmaceutical company responsible for medication, and organ dysfunction.

10. The method of claim 1, further comprising the step of relating said source characteristics to a combination of said source characteristics pertaining to said source patient.

11. The method of claim 1, wherein the information system is a standard ODBC compliant database.

12. The method of claim 1, wherein the information system can be imported into other preexisting databases.

13. A method for modifying medical protocols in an information system, comprising the steps of: entering elements of a medical protocol specific to a particular patient in an information system file pertaining to said patient; transmitting said file to a monitoring device of said patient; converting said file into a series of queries; and
communicating responses gathered by said queries back to a device for report-generation to medical personnel.

14. The method of claim 13, further comprising the step of:
communicating said responses to said database.

15. The method of claim 13, further comprising the step of:
remotely entering said elements of a medical protocol from one or more of the following: said monitoring device; cellular telephones; pagers; interactive televisions; custom-manufactured medical monitoring devices; separate personal computers; and “thin-client” Internet terminals.

16. The method of claim 13, further comprising the step of:
remotely entering said elements of a medical protocol by one or more of the following means: direct dial-in, and Internet access.

17. The method of claim 13, further comprising the step of:
relating said elements of a medical protocol to one or more of the following: medication type; medication dosage, dietary regimen, and reminders.

18. The method of claim 13, further comprising the step of:
relating said elements of a medical protocol to any algorithm-driven events that are based upon data entered by an information system user into the information system.

19. The method of claim 13, wherein the information system is a standard ODBC compliant database.

20. A method for providing services involving the use of remote devices communicating with a database comprising: providing a database containing data, including one or more inputted transaction terms for one or more users of remote devices linked to the database; providing one or more remote devices capable of communicating with said database; communicating data residing in the database to one or more of said devices; communicating between said devices and the database for an authorized period of time; whereby the transaction terms determine the authorized period of time of communication; and whereby the transaction terms are agreed upon by a customer and the provider of the database and communication services; and whereby at least a portion of the communicated data is stored in the database, and is specifically related to the user of the device.

21. A method for providing medical treatment protocols stored in a database which is part of a patient information system comprising:
providing a database containing medical protocols for one or more patients; providing two or more remote patient devices capable of communicating with said database; said patients interacting with said two or more remote devices in response to a series of messages provided to the patient by said devices; communicating said patient interactions from said devices to the database; providing rules that correlate the information communicated to the database with financial transaction terms to determine whether said communication is continued or discontinued.

22. A method for assigning a suite of software applications stored in a database comprising: providing a database containing one or more software applications; providing a device capable of communicating with said database; communicating with the database, to select one or more software applications based upon a specific device user characteristic; making said software application available to be used on the information device of a user based upon one or more characteristics of the user; downloading said software application to said device, or enabling said device to use the application via a client-server mechanism; the device user using said software to modify his or her behavior or life experience.

23. An apparatus for storing the medication of a patient comprising:
providing one or more medication containers of different sizes;
providing different functional features for each container; providing means whereby the container may communicate with an information device; whereby said container communicates information to said information device, to include information about the medication use of the patient.

24. A method for providing medication consumption information stored in a database comprising: providing a database; providing remote patient devices capable of communicating with said database, and capable of storing medication consumption information; communicating said medication consumption information to said database using said device; using said database to communicate said medication consumption information to one or more suppliers along the pharmaceutical supply chain.

25. The method of claim 24 wherein said remote patient device is selected from the group comprising Medi-Monitor, Med-eMonitor, Palm Pilot, portable telephone, mobile interactive pager, interactive television, or personal digital assistant.

26. A method for providing analysis of the effectiveness of a pharmaceutical compound comprising: providing a database containing one or more types of medical data for one or more patients, said data captured in real-time from the patient using one or more patient monitoring devices; whereby at least of portion of said data relates to the record of medication taken by said patients; whereby said record of medication is correlated with at least one additional data element related to the health of the patient.

27. A method for providing medical treatment protocols resident in a database comprising: providing a database containing one or more medical protocols for one or more patients; providing a patient device capable of communicating with said database; communicating to said device the medical treatment protocol resident in the database; using said patient device to communicate to the patient or caregiver said medical treatment protocol by electronic communication means, wherein said medical treatment protocol is converted into a sequence of specific events delivered in a specific order and at specific times; whereby the medical treatment protocol is converted into a sequence of prompt-and-record messages communicated by the device to the patient or caregiver

28. A method for providing medical treatment protocols stored in a database which is part of a patient information system comprising:
providing a database containing medical protocols for one or more patients; providing a remote patient device capable of communicating with said database; said patient interacting with said remote device in response to a series of messages provided to the patient by said device; communicating said patient interactions from said device to the database; providing rules that determine whether and how users of the information system may access the database to enter data representing the medical treatment protocol.

29. A method for providing medical treatment protocols stored in a database which is part of a patient information system comprising:
providing a database containing medical protocols for one or more patients; providing a remote patient device capable of communicating with said database; said patient interacting with said remote device in response to a series of messages provided to the patient by said device; communicating said patient interactions from said device to the database; providing rules that determine whether and how users of the information system may read the communications from said device to said database.

30. A method for providing medical treatment protocols stored in a database which is part of a patient information system comprising:
providing a database containing medical protocols for one or more patients; providing a remote patient device capable of communicating with said database; said patient interacting with said remote device in response to a series of messages provided to the patient by said device; communicating said patient interactions from said device to the database; providing rules that determine which information services provided by the database may be accessed by the patient.

31. A method for providing medical treatment protocols resident in a database comprising: providing a database containing one or more medical protocols for one or more patients; providing two or more patient devices capable of communicating with said database; communicating to said two or more devices the medical treatment protocol resident in the database; using said patient devices to communicate to the patient or caregiver said medical treatment protocol by electronic communication means, wherein said medical treatment protocol is converted into a sequence of specific events delivered in a specific order and at specific times; whereby the medical treatment protocol is converted into a sequence of prompt-and-record messages synchronously communicated by the two or more devices to the patient or caregiver

32. A method for providing medical treatment protocols stored in a database which is part of a patient information system comprising:
providing a database containing medical protocols for one or more patients; providing two or more remote patient devices capable of communicating with said database; said patient interacting with said two or more remote devices in response to a series of messages provided to the patient by said devices; communicating said patient interactions from said devices to the database; providing rules that synchronize the information communicated to the database such that the database is updated, and communicates updated information to the two or more patient monitors.

33. A method for providing medical treatment protocols stored in a database which is part of a patient information system comprising:
providing a database containing medical protocols for one or more patients; providing two or more remote patient devices capable of communicating with said database; said patient interacting with a selected one of said remote devices in response to a series of messages provided to the patient by said device; communicating said patient interactions from said device to the database; providing rules that update said data in said database such that future communications from said database to additional patient monitors reflect the updated data.

33. A method for providing medical treatment protocols stored in a database which is part of a patient information system comprising:
providing a database containing medical protocols for one or more patients; providing two or more remote patient devices used by a single patient capable of communicating with said database; said patient interacting with said two or more remote devices in response to a series of messages provided to the patient by said devices; communicating said patient interactions from said two or more devices to the database; providing rules that enable the database to properly synchronize the operation of the two or more devices such that the patient can use a suite of monitoring devices whereby the instructions for the proper operation of the suite of devices is continuously updated by the database

35. A method for providing icon-driven screens displayed on a device and stored in a database comprising: providing a database containing medical protocols for one or more patients; providing a remote patient device capable of communicating with said database; communicating using said device with the database; using said database to communicate to said device one or more icons to be displayed on said patient device screen, whereby selecting an icon displayed on said screen provides a link to medical information related to the patient.

36. The method of claim 35 wherein said device is selected from the group comprising Medi-Monitor, Med-eMonitor, Palm Pilot, portable telephone, mobile interactive pager, interactive television, or personal digital assistant.

37. A method for modifying medical protocols stored in a database comprising: providing a database containing medical protocols for three or more patients; providing a patient device capable of communicating with said database; communicating using said device with the database to select a group containing two or more patients based upon a specific patient characteristic; using said patient device to modify the group of selected patient protocols by electronic communication means wherein each protocol in the selected group is modified in the same way.
